(19) United States
(12) Patent Application Publication
(10) Pub. No.: US 2026/0344285 A1
(43) Pub. Date: Oct. 29, 2026

(54) ORGANIC COMPOUND AND ORGANIC LIGHT EMITTING ELEMENT

(71) Applicant: CANON KABUSHIKI KAISHA, Tokyo (JP)

(72) Inventors: KAORI FUJISAWA, Fukuoka (JP); AKIRA TSUBOYAMA, Tokyo (JP); KATSUYUKI HOTTA, Kanagawa (JP); HIDEYUKI SHIRAHASE, Osaka (JP); SEIYA MIZUNOYA, Kanagawa (JP)

(21) Appl. No.: 19/462,851

(22) Filed: Jan. 28, 2026

(30) Foreign Application Priority Data
Feb. 5, 2025 (JP) 2025-017637

(51) Int. Cl. H10K 85/60; C07D 401/14; C07D 491/048; C09D 11/00; H10K 50/11; H10K 59/10

(52) U.S. Cl. CPC H10K 85/657; C07D 401/14; C07D 491/048; C09D 11/00; H10K 50/11; H10K 59/10; H10K 85/6572

(57) ABSTRACT

An organic compound is represented by formula (1) and has a substituent represented by formula (2-1) or (2-2).

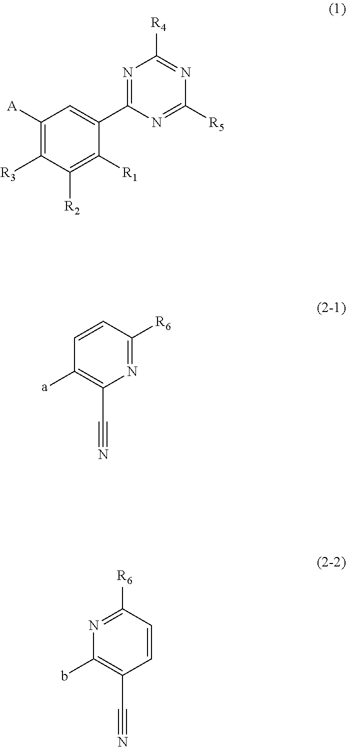

### BACKGROUND

### Field of the Technology

The present disclosure relates to an organic compound, an organic light emitting element including the organic compound, and an apparatus including the organic light emitting element.

### Description of the Related Art

An organic light emitting element (hereinafter may also be referred to as an “organic electroluminescent element” or an “organic EL element”) is an electronic element that includes a first electrode, a second electrode, and an organic compound layer disposed between the electrodes. The pair of electrodes inject electrons and holes to generate excitons of a light-emitting organic compound in the organic compound layer, and the organic EL element emits light as the excitons return to the ground state.

A delayed fluorescence material (thermally activated delayed fluorescence (TADF) material) usually assumes a structure in which a donor moiety and an acceptor moiety are linked (hereinafter, this structure is referred to as “DA type” structure). A DA-type TADF material in its excited state undergoes reverse intersystem crossing (RISC) from the excited triplet state to the excited singlet state, and then emits fluorescence as the material returns from the excited singlet state to the ground state. In other words, with a TADF material, not only the excited singlet state but also the excited triplet state can be utilized in the fluorescence emission through the reverse intersystem crossing channel, and thus light emission efficiency higher than that of typical fluorescent materials can be achieved.

Ever since this principle was found, various TADF materials have been discovered through various studies. International Publication No. 2023/140374 (PTL 1) discloses a compound A having the following structure:

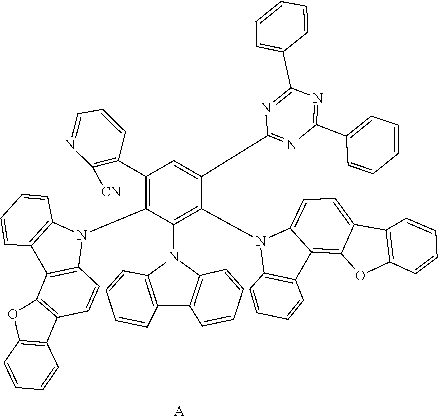

### SUMMARY

There is provided an organic compound represented by formula (1).

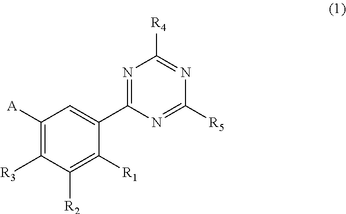

In formula (1), A is a group represented by formula (2-1) or (2-2), R₁ is a group represented by formula (3) or (4-1), R₂ is a substituted or unsubstituted alkyl group, a substituted or unsubstituted aryl group, a substituted or unsubstituted heteroaryl group, or a group represented by formula (2-1) or (2-2), R₃ is a hydrogen atom or a group represented by formula (3), when R₂ is a group represented by formula (2-1) or (2-2), R₁ is a group represented by formula (3) or (4-1) and R₃ is a hydrogen atom, when R₂ is an alkyl group, an aryl group, or a heteroaryl group, R₁ is a group represented by formula (3) and R₃ is a hydrogen atom or a group represented by formula (3), R₄ and R₅ are each independently a substituted or unsubstituted aryl group, a substituted or unsubstituted alkyl group, or a substituted or unsubstituted heteroaryl group,

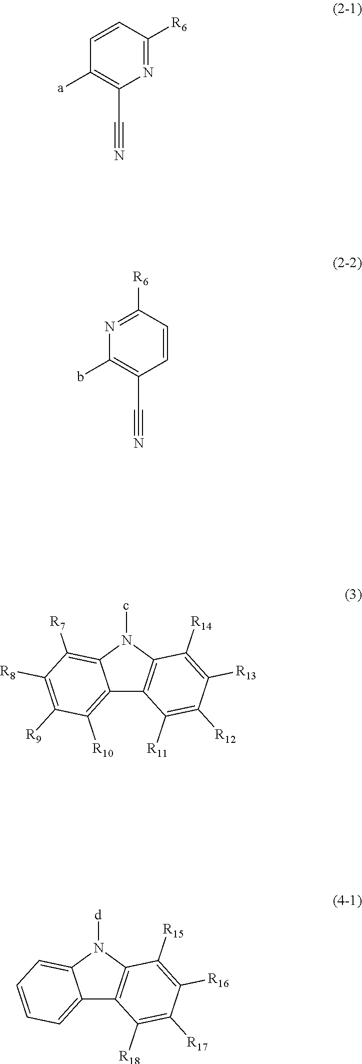

in formulae (2-1) and (2-2), R₆ is a substituted or unsubstituted alkyl group, a substituted or unsubstituted aryl group, or a substituted or unsubstituted heteroaryl group, in formula (3), R₇ to R₁₄ are each independently selected from a hydrogen atom, a deuterium atom, a halogen atom, a substituted or unsubstituted alkyl group, a substituted or unsubstituted aryl group, a substituted or unsubstituted heterocyclic group, a substituted or unsubstituted amino group, a substituted or unsubstituted alkoxy group, a substituted or unsubstituted aryloxy group, a substituted or unsubstituted heteroaryloxy group, a substituted or unsubstituted silyl group, and a cyano group, in formula (3), among R₁₁ to R₁₄, R₁ and R₁₂, R₁₂ and R₁₃, or R₁₃ and R₁₄ optionally bond to * in formula (4-2),

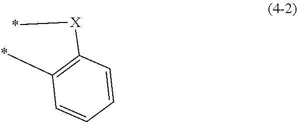

in formula (4-2), X is selected from an oxygen atom, a sulfur atom, a selenium atom, and a tellurium atom, and in formulae (2-1), (2-2), (3), and (4-1), a, b, c, and d each represent a bonding site to formula (1).

Features of the present disclosure will become apparent from the following description of embodiments with reference to the attached drawings. The following description of embodiments is described by way of example.

### BRIEF DESCRIPTION OF THE DRAWINGS

FIG. 1A is a schematic cross sectional view of one example of pixels of a display apparatus according to an embodiment of the present disclosure. FIG. 1B is a schematic cross sectional view of one example of a display apparatus that uses an organic EL element according to an embodiment of the present disclosure.

FIG. 2 is a schematic diagram of one example of a display apparatus according to one embodiment of the present disclosure.

FIG. 3A is a schematic diagram of one example of an imaging apparatus according to an embodiment of the present disclosure. FIG. 3B is a schematic diagram of one example of electronic equipment according to an embodiment of the present disclosure.

FIG. 4A is a schematic diagram of one example of a display apparatus according to an embodiment of the present disclosure. FIG. 4B is a schematic diagram of one example of a foldable display apparatus.

FIG. 5A is a schematic diagram of one example of lighting apparatus according to an embodiment of the present disclosure. FIG. 5B is a schematic diagram of an automobile that includes a vehicle lighting section according to an embodiment of the present disclosure.

FIG. 6A is a schematic diagram of one example of a wearable device according to an embodiment of the present disclosure. FIG. 6B is a schematic diagram of one example of a wearable device according to an embodiment of the present disclosure equipped with an imaging apparatus.

FIG. 7A is a schematic diagram of one example of an image forming apparatus according to an embodiment of the present disclosure.

FIG. 7B is a schematic diagram of one example of an exposure light source of an image forming apparatus according to an embodiment of the present disclosure. FIG. 7C is a schematic diagram of one example of an exposure light source of an image forming apparatus according to an embodiment of the present disclosure.

FIG. 8 illustrates general formula (5) representing a structure of an organic compound.

### DESCRIPTION OF THE EMBODIMENTS

According to the studies conducted by the inventors, the compound A disclosed in PTL 1 has a small singlet-triplet energy gap AEST but has a molecular weight as high as 1087; thus, the sublimation temperature is high and there is anticipated a high possibility of decomposition by sublimation, for example.

To address this disadvantage, the present disclosure provides an organic compound that has a small AEST and a low sublimation temperature.

The organic compound of the present disclosure is a compound used in an organic light emitting element and has a specific structure. The structure is described below in detail.

In this description, examples of the halogen atom include, but are not limited to, a fluorine atom, a chlorine atom, a bromine atom, and an iodine atom.

An alkyl group may have 1 or more and 40 or less carbon atoms, 1 or more and 20 or less carbon atoms, 1 or more and 10 or less carbon atoms, 1 or more and 6 or less carbon atoms, or 1 or more and 4 or less carbon atoms. Specific examples of the alkyl group include, but are not limited to, a methyl group, an ethyl group, an n-propyl group, an isopropyl group, an n-butyl group, a t-butyl group, an s-butyl group, a pentyl group, a hexyl group, an octyl group, a cyclohexyl group, a t-pentyl group, and a 3-methylpentan-3-yl group.

An aryl group may have 6 or more and 20 or less carbon atoms, 6 or more and 18 or less carbon atoms, 6 or more and 15 or less carbon atoms, or 6 or more and 12 or less carbon atoms. Specific examples of the aryl group include, but are not limited to, a phenyl group, a naphthyl group, an indenyl group, a biphenyl group, a terphenyl group, a fluorenyl group, a phenanthryl group, a triphenylenyl group, a pyrenyl group, an anthracenyl group, a perylenyl group, a chrysenyl group, and a fluoranthenyl group.

A heterocyclic group may have 3 or more and 24 or less carbon atoms, 3 or more and 18 or less carbon atoms, 3 or more and 12 or less carbon atoms, or 5 or more and 12 or less carbon atoms. Specific examples of the heterocyclic group include, but are not limited to, a pyridyl group, a pyrimidyl group, a pyrazyl group, a triazyl group, a benzofuranyl group, a benzothiophenyl group, a dibenzofuranyl group, a dibenzothiophenyl group, an oxazolyl group, an oxadiazolyl group, a thiazolyl group, a thiadiazolyl group, a carbazolyl group, an acridinyl group, and a phenanthrolyl group.

An amino group may be a substituted amino group substituted with an alkyl group or an aryl group or a substituted amino group substituted with an alkyl group having 1 or more and 4 or less carbon atoms or with an aryl group having 6 or more and 12 or less carbon atoms. Specific examples of the amino group include, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-t-tributylphenyl)amino group, an N-phenyl-N-(4-trifluoromethylphenyl)amino group, and an N-piperidyl group.

An alkoxy group may have 1 or more and 40 or less carbon atoms, 1 or more and 20 or less carbon atoms, 1 or more and 10 or less carbon atoms, 1 or more and 6 or less carbon atoms, or 1 or more and 4 or less carbon atoms. Specific examples of the alkoxy group include, but are not limited to, a methoxy group, an ethoxy group, a propoxy group, a 2-ethyl-octyloxy group, and a benzyloxy group.

A specific example of an aryloxy group is a phenoxy group, but the aryloxy group is not limited to this.

A specific example of a heteroaryloxy group is a thioenyloxy group, but the heteroaryloxy group is not limited to this.

A silyl group is a group in which a silicon atom has a substituent. This substituent may be a substituted or unsubstituted alkyl group or a substituted or unsubstituted aryl group. The substituted or unsubstituted alkyl group that the silicon atom has may be a substituted or unsubstituted alkyl group having 1 or more and 4 or less carbon atoms. The substituted or unsubstituted aryl group that the silicon atom has may be a substituted or unsubstituted aryl group having 6 or more and 10 or less carbon atoms. The silyl group may be a trialkylsilyl group or a triarylsilyl group. Specific examples of the silyl group include, but are not limited to, a trimethylsilyl group and a triphenylsilyl group.

Examples of the substituents that the alkyl group, the aryl group, the heterocyclic group, the amino group, the alkoxy group, the aryloxy group, the heteroaryloxy group, and the silyl group may have include, but are not limited to, halogen atoms such as fluorine, chlorine, bromine, and iodine; alkyl groups such as a methyl group, an ethyl group, an n-propyl group, an isopropyl group, an n-butyl group, and a t-butyl group; alkoxy groups such as a methoxy group, an ethoxy group, and a propoxy group; amino groups such as a dimethylamino group, a diethylamino group, a dibenzylamino group, a diphenylamino group, and a ditolylamino group; aryloxy groups such as a phenoxy group; aryl groups such as a phenyl group and a biphenyl group; heterocyclic groups such as a pyridyl group and a pyrrolyl group; and a cyano group.

### Organic Compound

First, the organic compound according to an embodiment is described. The organic compound according to this embodiment is an organic compound represented by general formula (1) below. The species of isotope of the hydrogen atoms intramolecularly present in the organic compound of the present embodiment is not particularly limited; for example, all hydrogen atoms in the molecule may be 1H or all or some of the hydrogen atoms may be 2H (deuterium). The organic compound of the present embodiment may be a compound that exhibits delayed fluorescence and may be a compound that exhibits a thermally active delayed fluorescence (TADF).

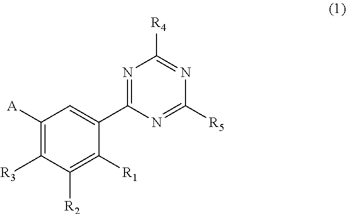

A is selected from groups represented by general formula (2-1) or (2-2) below.

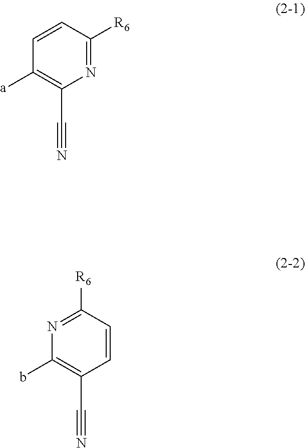

Of general formulae (2-1) and (2-2), (2-1) gives a smaller ΔEST.

R₁, R₂, and R₃

R₁ is a group represented by general formula (3) or (4-1) below:

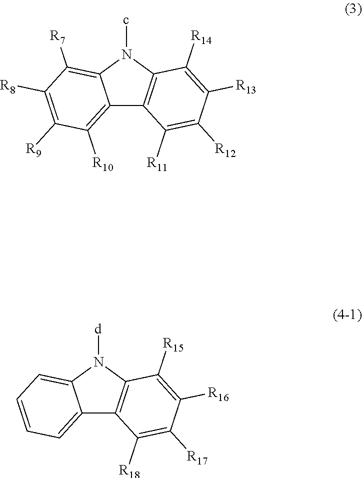

In general formula (3), among R₁₁ to R₁₄, R₁₁ and R₁₂, R₁₂ and R₁₃, or R₁₃ and R₁₄ may bond to * in general formula (4-2) below.

In general formula (4-1), among R₁₅ to R₁₈, R₁₅ and R₁₆, R₁₆ and R₁₇, or R₁₇ and R₁₈ may bond to * in general formula (4-2) below and the rest may be hydrogen atoms.

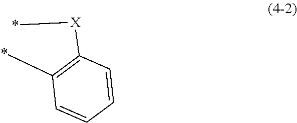

R₂ is a substituted or unsubstituted alkyl group, a substituted or unsubstituted aryl group, a substituted or unsubstituted heteroaryl group, or a group represented by general formula (2-1) or (2-2).

R₃ is a hydrogen atom or a group represented by general formula (3) above.

Here, when R₂ is a group represented by general formula (2-1) or (2-2), R₁ is a group represented by general formula (3) or (4-1) and R₃ is a hydrogen atom.

When R₂ is an alkyl group, an aryl group, or a heteroaryl group and when R₁ is a group represented by general formula (3), R₃ is a hydrogen atom or a group represented by general formula (3).

When R₁ and R₃ are groups represented by general formula (3), R₁ and R₃ may each be any of groups represented by the following structural formulae from the viewpoint of the molecular weight.

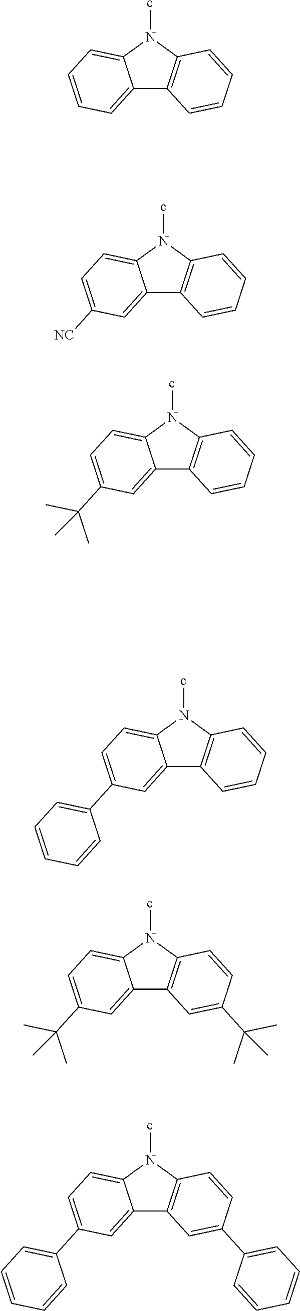

R₄, R₅, and R₆

In general formulae (1) and (2), R₄ to R₆ are each independently substituted with a substituted or unsubstituted aryl group, a substituted or unsubstituted alkyl group, or a substituted or unsubstituted heteroaryl group.

When R₄ to R₆ are each an alkyl group having 1 or more and 4 or less carbon atoms or an aryl group having 6 or more and 12 or less carbon atoms, the organic compound of the present embodiment exhibits a low molecular weight and excellent sublimability.

R₄ to R₆ may each be an aryl group having 6 or more and 12 or less carbon atoms, may be an aryl group having 6 or more and 10 or less carbon atoms, or may be a phenyl group.

R₇ to R₁₄

In general formula (3), R₇ to R₁₄ are each independently selected from a hydrogen atom, a deuterium atom, a halogen atom, a substituted or unsubstituted alkyl group, a substituted or unsubstituted aryl group, a substituted or unsubstituted heterocyclic group, a substituted or unsubstituted amino group, a substituted or unsubstituted alkoxy group, a substituted or unsubstituted aryloxy group, a substituted or unsubstituted heteroaryloxy group, a substituted or unsubstituted silyl group, and a cyano group.

R₇ to R₁₄ may each be a hydrogen atom, a deuterium atom, a substituted or unsubstituted alkyl group having 1 or more and 4 or less carbon atoms, a substituted or unsubstituted aryl group having 6 or more and 12 or less carbon atoms, a substituted or unsubstituted heteroaryl group having 6 or more and 12 or less carbon atoms, or a cyano group.

R₄ to R₁₄ may each be a substituted or unsubstituted alkyl group having 1 or more and 4 or less carbon atoms, a substituted or unsubstituted aryl group having 6 or more and 14 or less carbon atoms, or a substituted or unsubstituted heteroaryl group having 6 or more and 12 or less from the viewpoint of sublimability, and may each be any of the groups represented by structural formulae below, where * in the structural formulae below represents a bonding site.

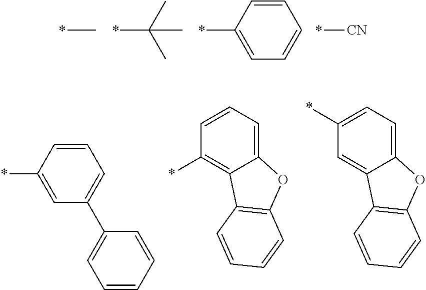

Here, when R₂ is an alkyl group having 1 or more and 4 or less carbon atoms or an aryl group having 6 carbon atoms, the organic compound of the present embodiment exhibits a low molecular weight and excellent sublimability. R₂ may be a methyl group, a t-butyl group, or a phenyl group.

In general formula (3), among R₁₁ to R₁₄, one set of adjacent two groups can bond to (4-2). The adjacent two groups are R₁₁ and R₁₂, R₁₂ and R₁₃, or R₁₃ and R₁₄.

In particular, when (4-2) bonds to R₁₁ and R₁₂, the element lifetime is excellent. Thus, from the viewpoint of the element lifetime, R₁₁ and R₁₂ can bond to * in (4-2).

R₁₅ to R₁₈

In general formula (4-1), among R₁₅ to R₁₈, one set of adjacent two groups may bond to (4-2), and the rest is substituted with hydrogen atoms. The adjacent two groups are R₁₅ and R₁₆, R₁₆ and R₁₇, or R₁₇ and R₁₈. In particular, when (4-2) bonds to R₁₇ and R₁₈, the element lifetime is excellent. Thus, from the viewpoint of the element lifetime, R₁₇ and R₁₈ can bond to * in (4-2).

### X

In general formula (4-2), X is selected from an oxygen atom, a sulfur atom, a selenium atom, and a tellurium atom. X may be an oxygen atom or a sulfur atom, or may be an oxygen atom.

a, b, c, and d

In general formulae (2-1), (2-2), (3), and (4-1), a, b, c, and d each represent the site that bonds to general formula (1). In general formula (4-2), each * represents a site that bonds to general formula (4-1).

### Features of Structure of Organic Compound

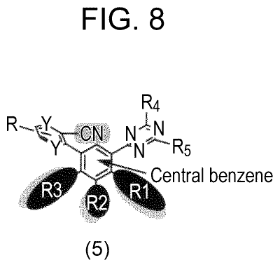

The organic compound of this embodiment has the following four features.
(a) Since the molecular weight of the skeletons represented by general formulae (2-1) and (2-2) are low, the sublimation temperature can be decreased.
(b) In the pyridine skeletons in general formulae (2-1) and (2-2), the cyano group is ortho to the bonding site to general formula (1), and thus general formula (1) and general formula (2-1) or (2-2) assume a twisted conformation.
(c) Since many substituents are introduced to the central benzene in general formula (1), the bond angle between the donor (carbazole) and the acceptor (benzene) becomes twisted.
(d) Y in general formula (5) in FIG. 8 is substituted with an N atom or CH. Moreover, the ortho position R with respect to the N atom is substituted with an alkyl group or an aryl group to improve the molecular stability.

Hereinafter, the features (a) to (d) above are described.
(a) Since the molecular weight of the skeletons represented by general formulae (2-1) and (2-2) are low, the sublimation temperature can be decreased.

In general, a molecule with a high molecular weight or a molecule with strong intermolecular interaction is known to have a high sublimation temperature due to its molecular weight or high energy required to sublimate the interaction. Since the material represented by general formula (5) has bulky substituents twisted with respect to the central benzene, the intermolecular interaction is easily inhibited. In other words, since the intermolecular interaction is small, the sublimation temperature becomes lower than that of a compound having the same molecular weight. Furthermore, due to the low molecular weight, a low sublimation temperature is achieved. Thus, the molecular weight of the organic compound of the present embodiment may be 950 or less.
(b) In the pyridine skeletons in general formulae (2-1) and (2-2), the cyano group is ortho to the bonding site to general formula (1), and thus general formula (1) and general formula (2-1) or (2-2) assume a twisted conformation.

Since the cyano group obstructs rotation of the cyanopyridine ring, the cyanopyridine ring assumes a twisted conformation with respect to the central benzene.

Since the cyanopyridine ring assumes a twisted conformation, the donor and the acceptor can also assume a twisted conformation even when hydrogen is introduced to R₃ to decrease the molecular weight. In other words, a larger bond angle is formed between the donor and the acceptor. As a result, orbital infiltration into the central benzene can be reduced. Thus, the HOMO-LUMO separation is improved, and ΔEST (the gap between the lowest excited singlet energy and the lowest excited triplet energy) is decreased. ΔEST may be 0.1 eV or less as a calculated value. Here, HOMO is the highest occupied molecular orbital, and LUMO is the lowest unoccupied molecular orbital.
(c) Since many substituents are introduced to the central benzene, the bond angle between the donor (carbazole) and the acceptor (benzene) becomes twisted.

By introducing many substituents (R₁ to R₃) to the central benzene in general formula (5), the bond angle between the donor (carbazole) and the acceptor (benzene) can be twisted. As a result, orbital infiltration into the central benzene can be reduced. Thus, HOMO-LUMO separation is improved, and ΔEST is decreased. ΔEST may be 0.1 eV or less as a calculated value.

In particular, by introducing an alkyl group or aryl group having a molecular weight of 200 or less into R₂, the low molecular weight can be maintained. From the viewpoint of decreasing the molecular weight, R₂ may be substituted with a substituent (alkyl group or aryl group) having a molecular weight of 100 or less.
(d) Y in general formula (5) is substituted with an N atom or CH. Moreover, the ortho position R with respect to the N atom is substituted with an alkyl group or an aryl group to improve the molecular stability.

It is generally known that the a position with respect to the nitrogen atom in the pyridine ring has a high electronic density, and the molecular stability is low. To address this, a substituent is introduced to the a position to decrease the electronic density of the a position. As a result, the reactivity of the molecule decreases, and the molecular stability is improved; thus, the element lifetime is improved.

Example compounds of the present disclosure are described below. The molecular weight and the calculation result of ΔEST for each of the compounds are indicated in Table 1.

### Method for Calculating ΔEST

To determine ΔEST between the levels, a geometry optimization calculation is performed to find the most stable structure of the ground state S₀ using the density functional method with the functional CAM-B3LYP and the basis set 6-31g**, and the excitation energy E_{S1} from S₀ to S₁ and the excitation energy E_{T1} from S₀ to T₁ are calculated with respect to the most stable structure of S₀ using a time-dependent density functional method with the aforementioned functional and basis set.

ΔEST with respect to T₁ can be determined by E_{S1}−E_{T1}.

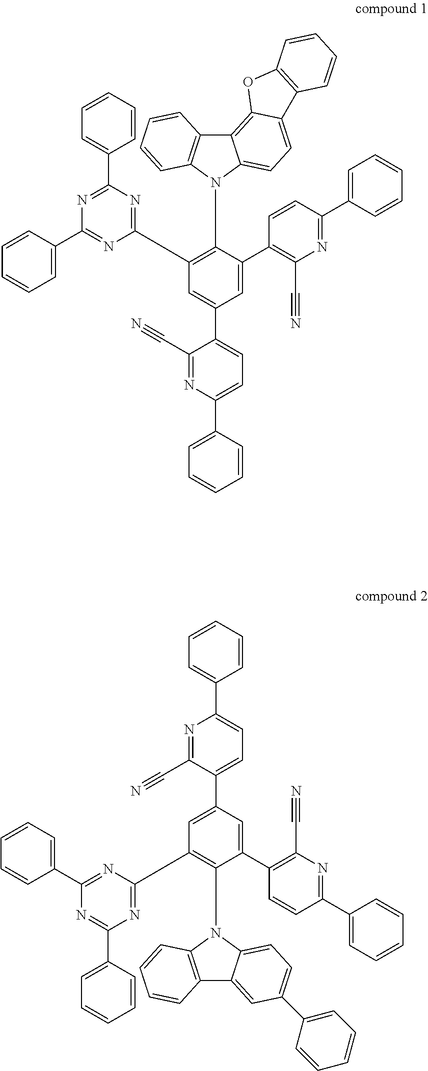 [SMILES: N#Cc1nc(-c2ccccc2)ccc1-c1cc(-c2nc(-c3ccccc3)nc(-c3ccccc3)n2)c(-n2c3ccccc3c3c4oc5ccccc5c4ccc32)c(-c2ccc(-c3ccccc3)nc2C#N)c1 ; N#Cc1nc(-c2ccccc2)ccc1-c1cc(-c2nc(-c3ccccc3)nc(-c3ccccc3)n2)c(-n2c3ccccc3c3cc(-c4ccccc4)ccc32)c(-c2ccc(-c3ccccc3)nc2C#N)c1]

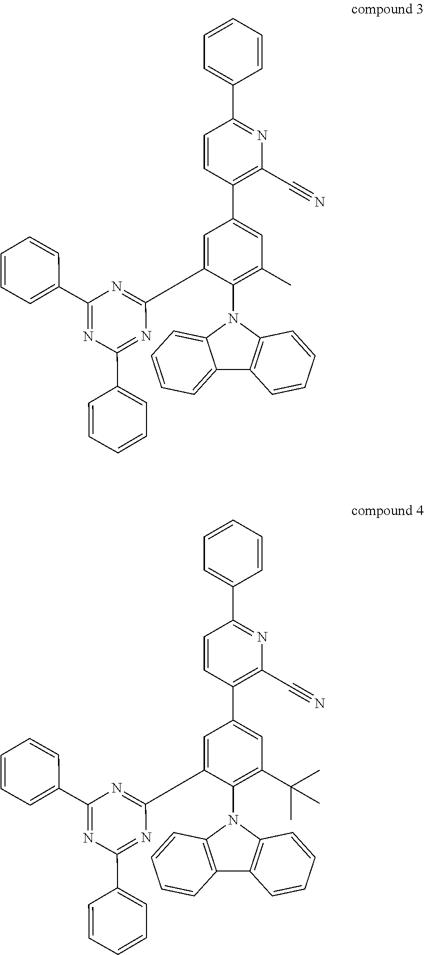 [SMILES: Cc1cc(-c2ccc(-c3ccccc3)nc2C#N)cc(-c2nc(-c3ccccc3)nc(-c3ccccc3)n2)c1-n1c2ccccc2c2ccccc21 ; CC(C)(C)c1cc(-c2ccc(-c3ccccc3)nc2C#N)cc(-c2nc(-c3ccccc3)nc(-c3ccccc3)n2)c1-n1c2ccccc2c2ccccc21]

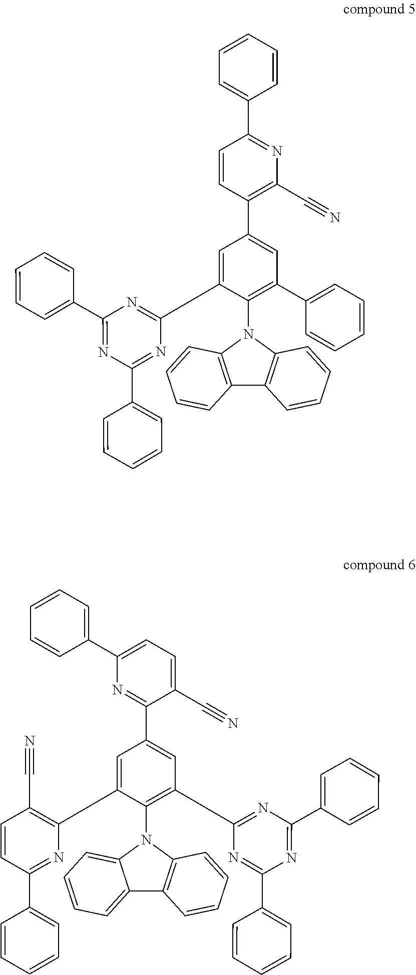 [SMILES: N#Cc1nc(-c2ccccc2)ccc1-c1cc(-c2ccccc2)c(-n2c3ccccc3c3ccccc32)c(-c2nc(-c3ccccc3)nc(-c3ccccc3)n2)c1 ; N#Cc1ccc(-c2ccccc2)nc1-c1cc(-c2nc(-c3ccccc3)nc(-c3ccccc3)n2)c(-n2c3ccccc3c3ccccc32)c(-c2nc(-c3ccccc3)ccc2C#N)c1]

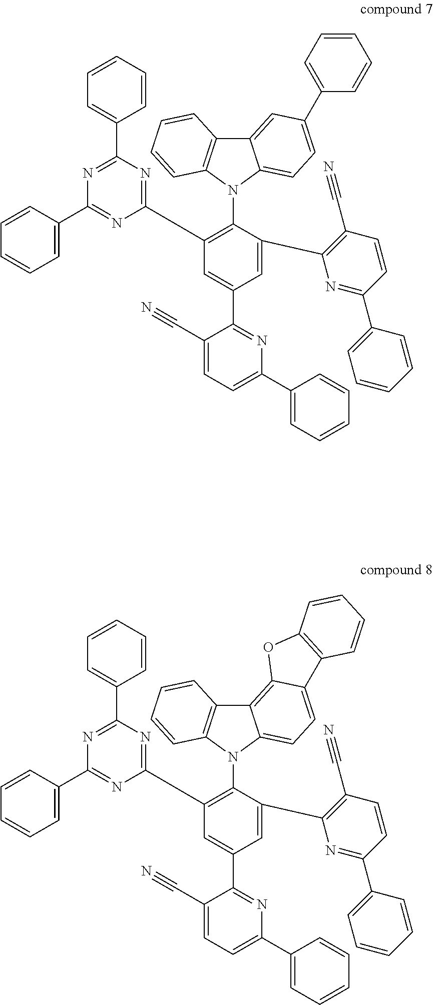 [SMILES: N#Cc1ccc(-c2ccccc2)nc1-c1cc(-c2nc(-c3ccccc3)nc(-c3ccccc3)n2)c(-n2c3ccccc3c3cc(-c4ccccc4)ccc32)c(-c2nc(-c3ccccc3)ccc2C#N)c1 ; N#Cc1ccc(-c2ccccc2)nc1-c1cc(-c2nc(-c3ccccc3)nc(-c3ccccc3)n2)c(-n2c3ccccc3c3c4oc5ccccc5c4ccc32)c(-c2nc(-c3ccccc3)ccc2C#N)c1]

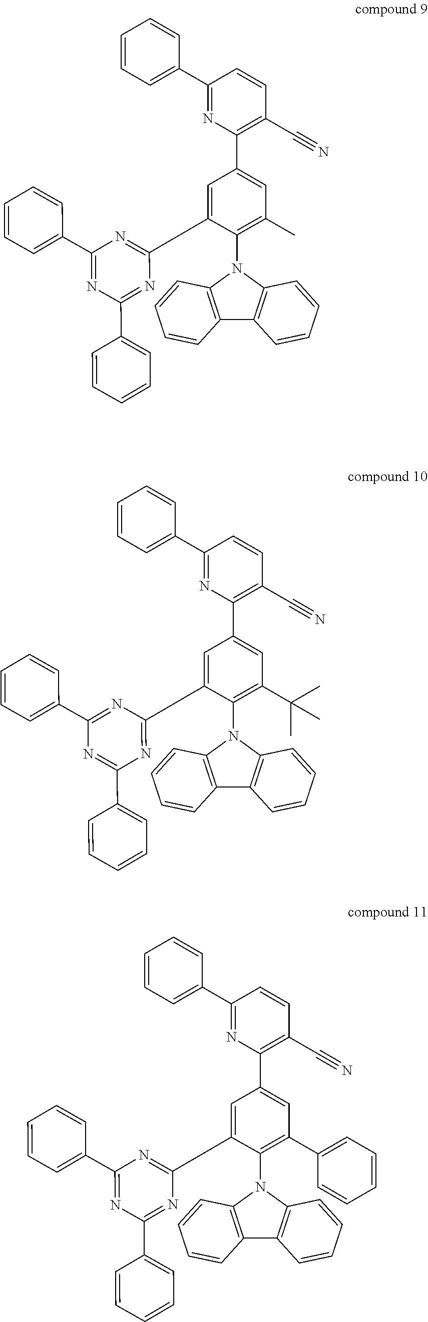 [SMILES: Cc1cc(-c2nc(-c3ccccc3)ccc2C#N)cc(-c2nc(-c3ccccc3)nc(-c3ccccc3)n2)c1-n1c2ccccc2c2ccccc21 ; CC(C)(C)c1cc(-c2nc(-c3ccccc3)ccc2C#N)cc(-c2nc(-c3ccccc3)nc(-c3ccccc3)n2)c1-n1c2ccccc2c2ccccc21 ; N#Cc1ccc(-c2ccccc2)nc1-c1cc(-c2ccccc2)c(-n2c3ccccc3c3ccccc32)c(-c2nc(-c3ccccc3)nc(-c3ccccc3)n2)c1]

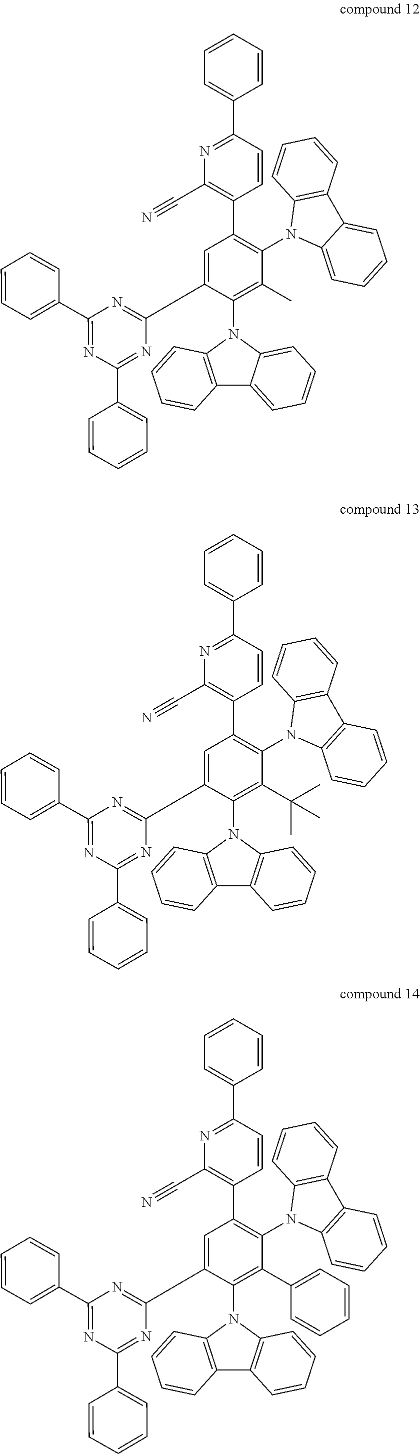 [SMILES: Cc1c(-n2c3ccccc3c3ccccc32)c(-c2nc(-c3ccccc3)nc(-c3ccccc3)n2)cc(-c2ccc(-c3ccccc3)nc2C#N)c1-n1c2ccccc2c2ccccc21 ; CC(C)(C)c1c(-n2c3ccccc3c3ccccc32)c(-c2nc(-c3ccccc3)nc(-c3ccccc3)n2)cc(-c2ccc(-c3ccccc3)nc2C#N)c1-n1c2ccccc2c2ccccc21 ; N#Cc1nc(-c2ccccc2)ccc1-c1cc(-c2nc(-c3ccccc3)nc(-c3ccccc3)n2)c(-n2c3ccccc3c3ccccc32)c(-c2ccccc2)c1-n1c2ccccc2c2ccccc21]

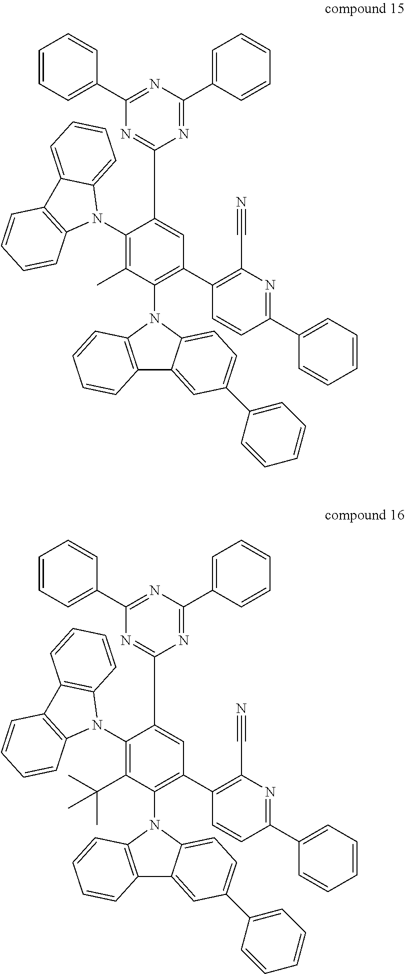 [SMILES: Cc1c(-n2c3ccccc3c3ccccc32)c(-c2nc(-c3ccccc3)nc(-c3ccccc3)n2)cc(-c2ccc(-c3ccccc3)nc2C#N)c1-n1c2ccccc2c2cc(-c3ccccc3)ccc21 ; CC(C)(C)c1c(-n2c3ccccc3c3ccccc32)c(-c2nc(-c3ccccc3)nc(-c3ccccc3)n2)cc(-c2ccc(-c3ccccc3)nc2C#N)c1-n1c2ccccc2c2cc(-c3ccccc3)ccc21]

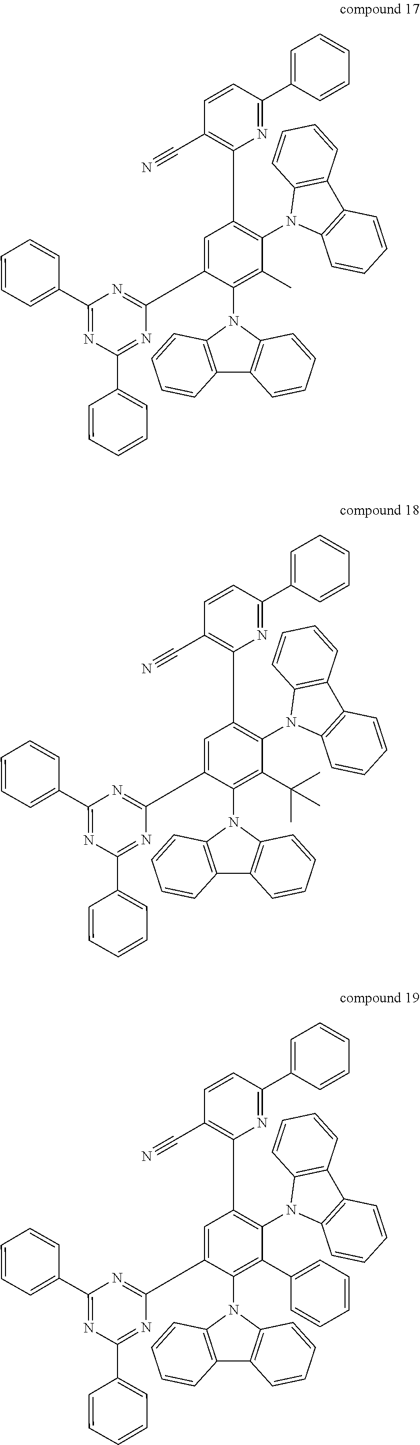 [SMILES: Cc1c(-n2c3ccccc3c3ccccc32)c(-c2nc(-c3ccccc3)nc(-c3ccccc3)n2)cc(-c2nc(-c3ccccc3)ccc2C#N)c1-n1c2ccccc2c2ccccc21 ; CC(C)(C)c1c(-n2c3ccccc3c3ccccc32)c(-c2nc(-c3ccccc3)nc(-c3ccccc3)n2)cc(-c2nc(-c3ccccc3)ccc2C#N)c1-n1c2ccccc2c2ccccc21 ; N#Cc1ccc(-c2ccccc2)nc1-c1cc(-c2nc(-c3ccccc3)nc(-c3ccccc3)n2)c(-n2c3ccccc3c3ccccc32)c(-c2ccccc2)c1-n1c2ccccc2c2ccccc21]

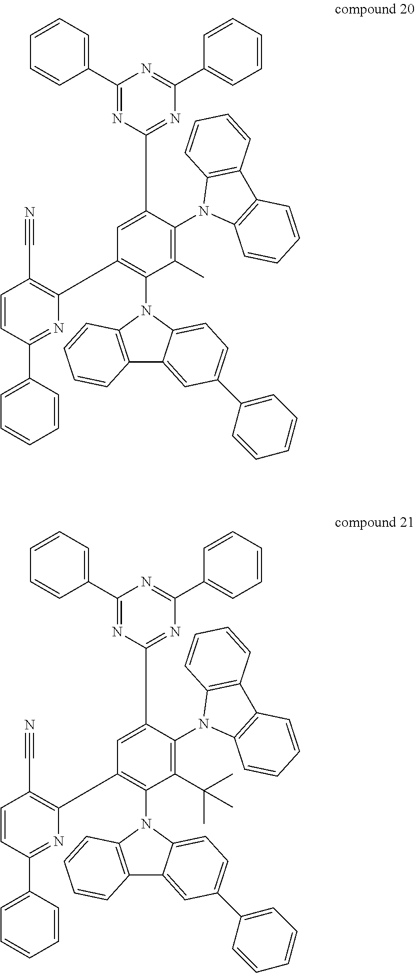 [SMILES: Cc1c(-n2c3ccccc3c3ccccc32)c(-c2nc(-c3ccccc3)nc(-c3ccccc3)n2)cc(-c2nc(-c3ccccc3)ccc2C#N)c1-n1c2ccccc2c2cc(-c3ccccc3)ccc21 ; CC(C)(C)c1c(-n2c3ccccc3c3ccccc32)c(-c2nc(-c3ccccc3)nc(-c3ccccc3)n2)cc(-c2nc(-c3ccccc3)ccc2C#N)c1-n1c2ccccc2c2cc(-c3ccccc3)ccc21]

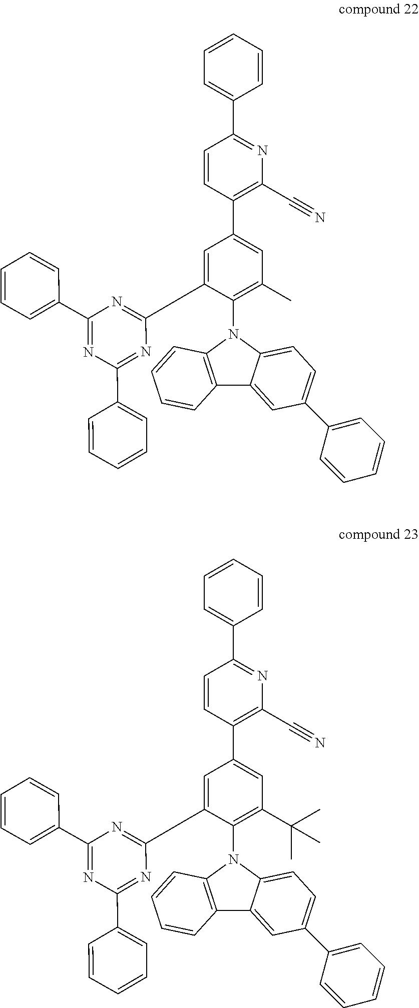 [SMILES: Cc1cc(-c2ccc(-c3ccccc3)nc2C#N)cc(-c2nc(-c3ccccc3)nc(-c3ccccc3)n2)c1-n1c2ccccc2c2cc(-c3ccccc3)ccc21 ; CC(C)(C)c1cc(-c2ccc(-c3ccccc3)nc2C#N)cc(-c2nc(-c3ccccc3)nc(-c3ccccc3)n2)c1-n1c2ccccc2c2cc(-c3ccccc3)ccc21]

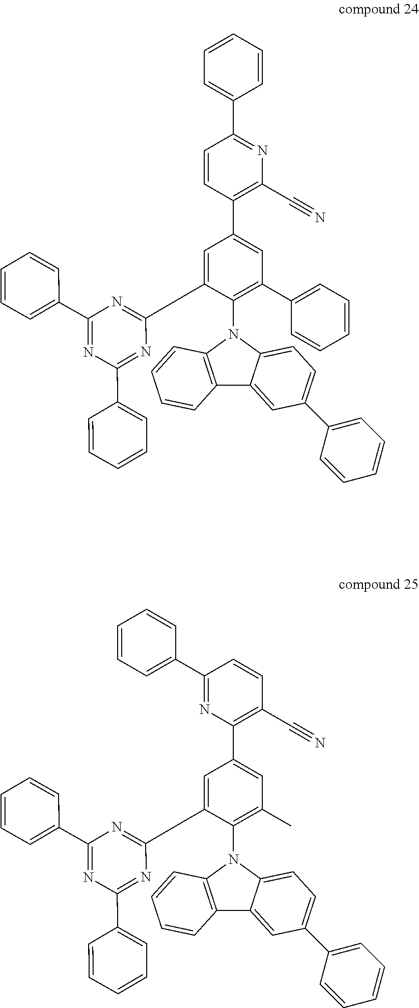 [SMILES: N#Cc1nc(-c2ccccc2)ccc1-c1cc(-c2ccccc2)c(-n2c3ccccc3c3cc(-c4ccccc4)ccc32)c(-c2nc(-c3ccccc3)nc(-c3ccccc3)n2)c1 ; Cc1cc(-c2nc(-c3ccccc3)ccc2C#N)cc(-c2nc(-c3ccccc3)nc(-c3ccccc3)n2)c1-n1c2ccccc2c2cc(-c3ccccc3)ccc21]

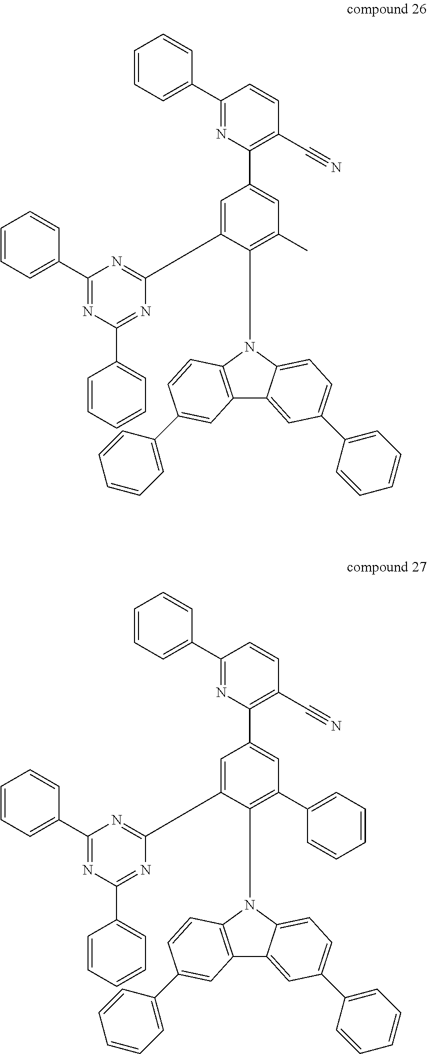 [SMILES: Cc1cc(-c2nc(-c3ccccc3)ccc2C#N)cc(-c2nc(-c3ccccc3)nc(-c3ccccc3)n2)c1-n1c2ccc(-c3ccccc3)cc2c2cc(-c3ccccc3)ccc21 ; N#Cc1ccc(-c2ccccc2)nc1-c1cc(-c2ccccc2)c(-n2c3ccc(-c4ccccc4)cc3c3cc(-c4ccccc4)ccc32)c(-c2nc(-c3ccccc3)nc(-c3ccccc3)n2)c1]

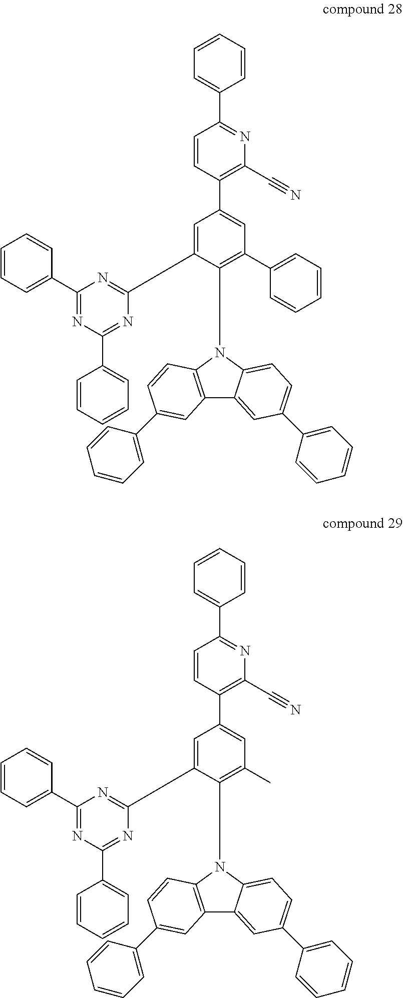 [SMILES: N#Cc1nc(-c2ccccc2)ccc1-c1cc(-c2ccccc2)c(-n2c3ccc(-c4ccccc4)cc3c3cc(-c4ccccc4)ccc32)c(-c2nc(-c3ccccc3)nc(-c3ccccc3)n2)c1 ; Cc1cc(-c2ccc(-c3ccccc3)nc2C#N)cc(-c2nc(-c3ccccc3)nc(-c3ccccc3)n2)c1-n1c2ccc(-c3ccccc3)cc2c2cc(-c3ccccc3)ccc21]

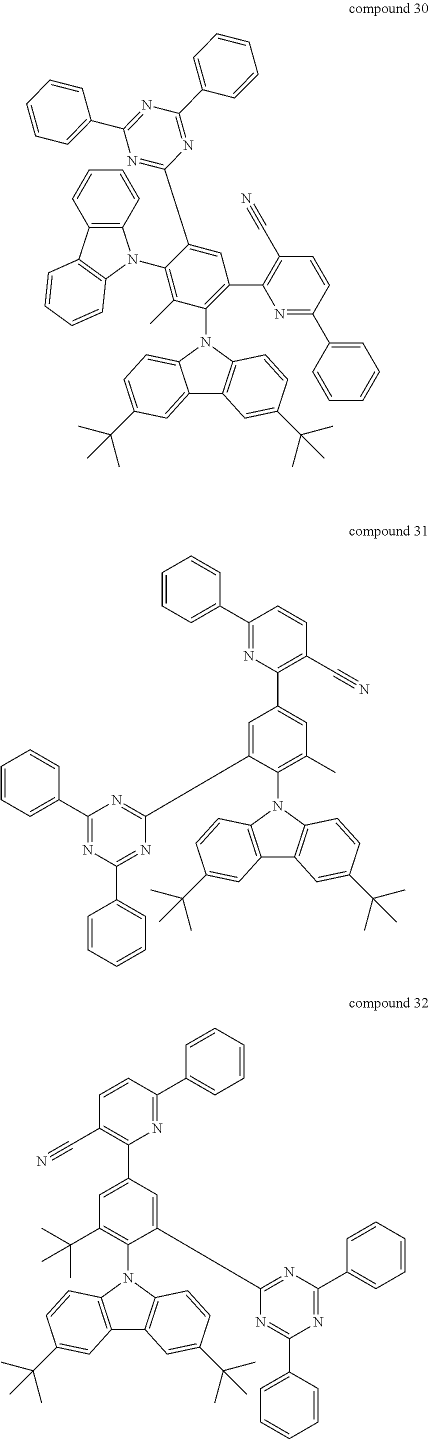

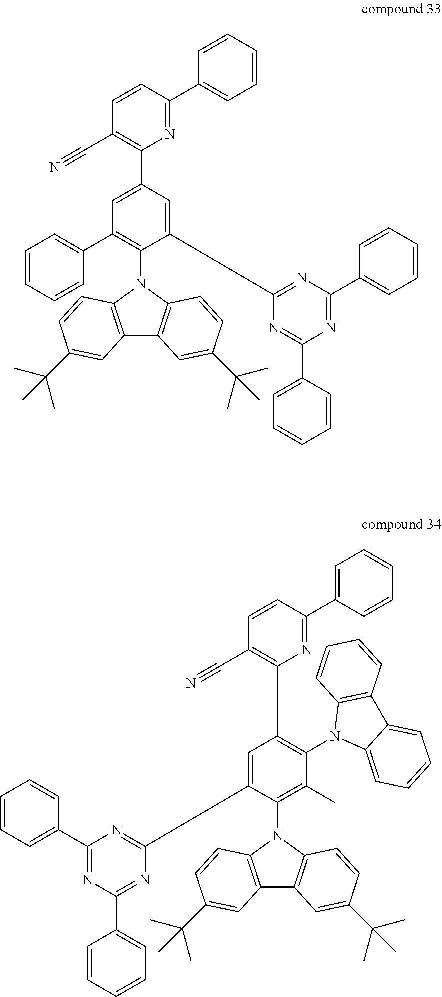 [SMILES: CC(C)(C)c1ccc2c(c1)c1cc(C(C)(C)C)ccc1n2-c1c(-c2ccccc2)cc(-c2nc(-c3ccccc3)ccc2C#N)cc1-c1nc(-c2ccccc2)nc(-c2ccccc2)n1 ; Cc1c(-n2c3ccc(C(C)(C)C)cc3c3cc(C(C)(C)C)ccc32)c(-c2nc(-c3ccccc3)nc(-c3ccccc3)n2)cc(-c2nc(-c3ccccc3)ccc2C#N)c1-n1c2ccccc2c2ccccc21]

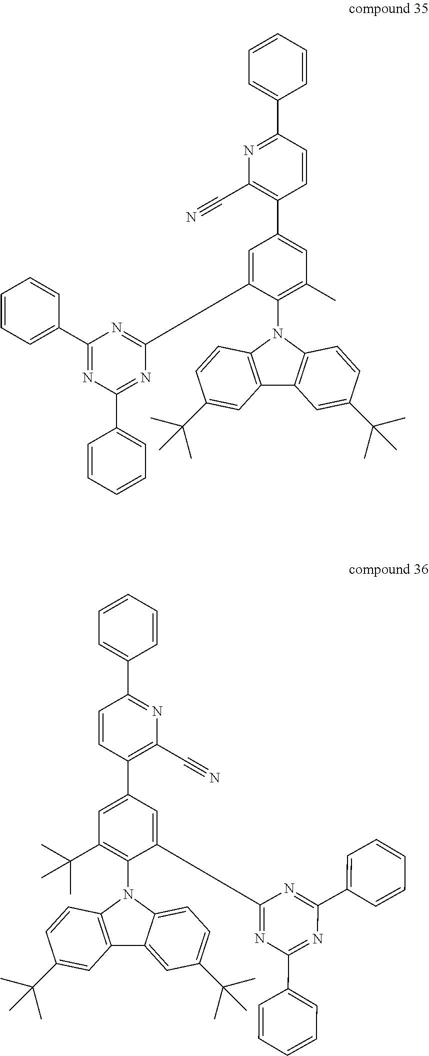

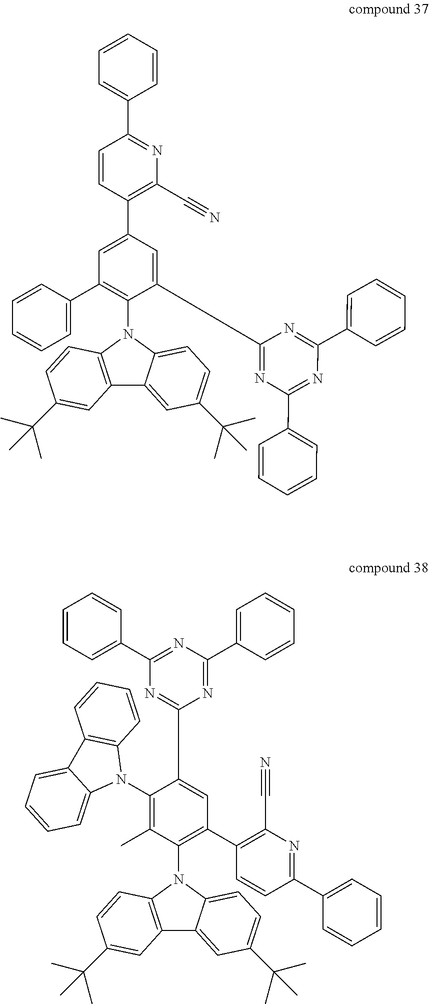 [SMILES: CC(C)(C)c1ccc2c(c1)c1cc(C(C)(C)C)ccc1n2-c1c(-c2ccccc2)cc(-c2ccc(-c3ccccc3)nc2C#N)cc1-c1nc(-c2ccccc2)nc(-c2ccccc2)n1 ; Cc1c(-n2c3ccccc3c3ccccc32)c(-c2nc(-c3ccccc3)nc(-c3ccccc3)n2)cc(-c2ccc(-c3ccccc3)nc2C#N)c1-n1c2ccc(C(C)(C)C)cc2c2cc(C(C)(C)C)ccc21]

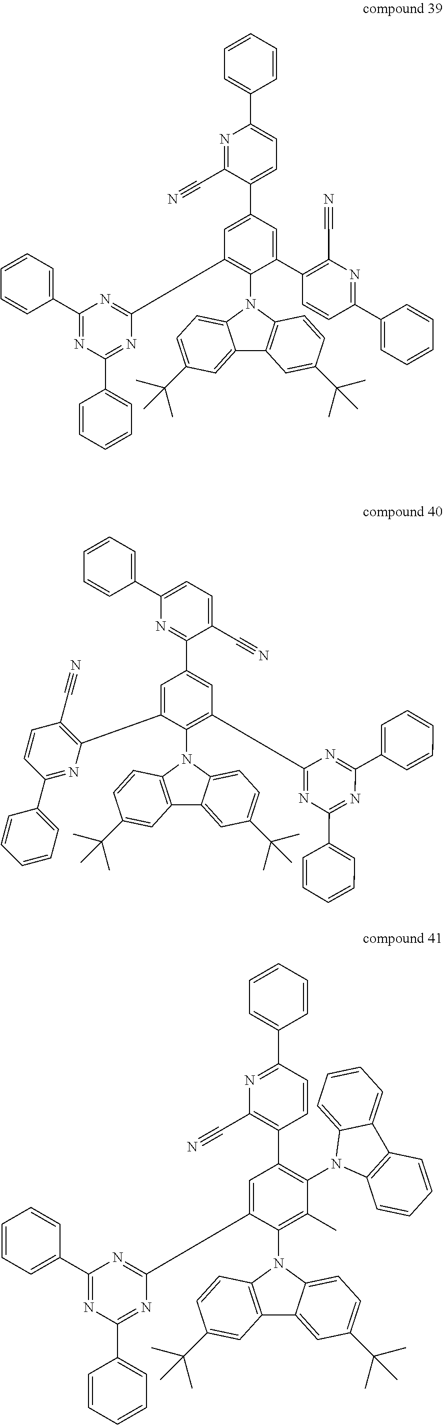 [SMILES: CC(C)(C)c1ccc2c(c1)c1cc(C(C)(C)C)ccc1n2-c1c(-c2nc(-c3ccccc3)nc(-c3ccccc3)n2)cc(-c2ccc(-c3ccccc3)nc2C#N)cc1-c1ccc(-c2ccccc2)nc1C#N ; CC(C)(C)c1ccc2c(c1)c1cc(C(C)(C)C)ccc1n2-c1c(-c2nc(-c3ccccc3)nc(-c3ccccc3)n2)cc(-c2nc(-c3ccccc3)ccc2C#N)cc1-c1nc(-c2ccccc2)ccc1C#N ; Cc1c(-n2c3ccc(C(C)(C)C)cc3c3cc(C(C)(C)C)ccc32)c(-c2nc(-c3ccccc3)nc(-c3ccccc3)n2)cc(-c2ccc(-c3ccccc3)nc2C#N)c1-n1c2ccccc2c2ccccc21]

TABLE 1

| Compound No. | Mw | ΔEST | Compound No. | Mw | ΔEST [eV] |
|---|---|---|---|---|---|
| 1 | 921 | 0.06 | 22 | 743 | 0.06 |
| 2 | 907 | 0.09 | 23 | 785 | 0.04 |
| 3 | 667 | 0.09 | 24 | 805 | 0.08 |
| 4 | 709 | 0.06 | 25 | 743 | 0.07 |
| 5 | 729 | 0.09 | 26 | 819 | 0.06 |
| 6 | 831 | 0.09 | 27 | 881 | 0.08 |
| 7 | 907 | 0.08 | 28 | 881 | 0.01 |
| 8 | 921 | 0.10 | 29 | 819 | 0.05 |
| 9 | 667 | 0.09 | 30 | 944 | 0.07 |
| 10 | 709 | 0.06 | 31 | 779 | 0.09 |
| 11 | 729 | 0.10 | 32 | 821 | 0.05 |
| 12 | 832 | 0.04 | 33 | 841 | 0.10 |
| 13 | 874 | 0.04 | 34 | 944 | 0.08 |
| 14 | 894 | 0.06 | 35 | 779 | 0.07 |
| 15 | 908 | 0.04 | 36 | 821 | 0.04 |
| 16 | 950 | 0.04 | 37 | 841 | 0.09 |
| 17 | 832 | 0.08 | 38 | 944 | 0.05 |
| 18 | 874 | 0.05 | 39 | 943 | 0.07 |
| 19 | 894 | 0.05 | 40 | 943 | 0.09 |
| 20 | 908 | 0.10 | 41 | 944 | 0.03 |
| 21 | 950 | 0.04 | | | |

Table 1 shows that, compared with the organic compounds of Comparative Examples described below, the organic compound of the present embodiment can achieve both the decrease in molecular weight and the narrowing of ΔEST. This is because the organic compound of the present embodiment has the features (a) to (d) above, and thus the orbital distributions of the HOMO and the LUMO can be efficiently separated.

### Organic Light Emitting Element

Next, an organic light emitting element according to the present embodiment is described. The organic light emitting element of the present embodiment includes at least a first electrode, a second electrode, and an organic compound layer disposed between these electrodes.

Of the first electrode and the second electrode, one is a positive electrode and the other is a negative electrode. In the organic light emitting element of the present embodiment, the organic compound layer may be a single layer or a laminate constituted by multiple layers so long as a light emitting layer is included. Here, when the organic compound layer is a laminate constituted by multiple layers, the organic compound layer may include, in addition to the light emitting layer, a hole injection layer, a hole transport layer, an electron blocking layer, a hole/exciton blocking layer, an electron transport layer, an electron injection layer, etc. In addition, the light emitting layer may be a single layer or a laminate constituted by multiple layers.

In the organic light emitting element of the present embodiment, at least one layer in the organic compound layer contains the organic compound of the present embodiment. Specifically, the organic compound of the present embodiment is contained in one of the aforementioned light emitting layer, hole injection layer, hole transport layer, electron blocking layer, hole/exciton blocking layer, electron transport layer, electron injection layer, etc. The organic compound of the present embodiment may be contained in the light emitting layer.

In the organic light emitting element of the present embodiment, when the organic compound of the present embodiment is contained in the light emitting layer, the light emitting layer may be a layer solely composed of the organic compound of the present embodiment or may be a layer composed of the organic compound of the present embodiment and other compounds such as a second organic compound and a third organic compound. The organic compound of the present embodiment in the light emitting layer may serve as a guest and dope the host material (second organic compound), or may serve as an assist dopant and dope the host material (second organic compound) together with a fluorescent material (third organic compound).

In a doped layer, the host is a compound that has the largest mass ratio among the compounds constituting the light emitting layer. In addition, the guest is a compound that has a mass ratio smaller than that of the host among the compounds constituting the light emitting layer and that is responsible for main light emission. Furthermore, the assist dopant is a compound that has a mass ratio smaller than that of the host among the compounds constituting the light emitting layer and that has a role of sensitizing the excitons.

When the organic compound of the present embodiment is used as a guest, the concentration of the organic compound of the present embodiment relative to the entire light emitting layer may be 0.01 mass % or more and 50 mass % or less or may be 10 mass % or more and 50 mass % or less. When the organic compound of the present embodiment is used as an assist dopant, the concentration of the organic compound of the present embodiment relative to the entire light emitting layer may be 1 mass % or more and 50 mass % or less or may be 10 mass % or more and 50 mass % or less. In this case, the concentration of the fluorescent material relative to the entire light emitting layer may be 0.01 mass % or more and 20 mass % or less or may be 0.01 mass % or more and 5 mass % or less.

The inventors of the present disclosure have conducted various studies and found that when the organic compound of the present embodiment is used as the host or guest of the light emitting layer, in particular, as the guest of the light emitting layer, an element that achieves highly efficient, high-luminance light output and that has high durability is obtained. This light emitting layer may be single-layered or multilayered, and it is possible to mix the emission color of the organic compound of the present embodiment with other colors by containing light emitting materials having other emission colors. The term “multilayered” means that a light emitting layer and other light emitting layers are laminated on top of each other. In such a case, the emission color of the organic light emitting element is not limited to the emission color of the organic compound of the present embodiment.

More specifically, the emission color may be white or an intermediate color. When the emission color is white, other light emitting layers emit light of colors other than the emission color of the organic compound of the present embodiment, for example, when the emission color of the organic compound of the present embodiment is blue, the colors of light emitted by the other light emitting layers are green and red. Furthermore, the method for forming the film is vapor deposition or coating.

The organic compound of the present embodiment can be used as a material constituting an organic compound layer other than the light emitting layer constituting the organic light emitting element of the present embodiment. Specifically, the organic compound may be used as a material constituting an electron transport layer, an electron injection layer, a hole transport layer, a hole injection layer, a hole blocking layer, or the like. In such a case, the emission color of the organic light emitting element is not limited to the emission color of the organic compound of the present embodiment. More specifically, white light or light of an intermediate color may be emitted.

### Other Compounds

If necessary, known low-molecular-weight and high-molecular-weight hole injection compounds or hole transport compounds, host compounds, light emitting compounds, electron injection compounds, electron transport compounds, etc., can be used in combination with the organic compound of the present embodiment. Examples of such compounds are as follows.

A hole injection/transport material may be a material that has high hole mobility to facilitate injection of holes from the positive electrode and transport of the injected holes to the light emitting layer. In order to suppress degradation of the film quality such as crystallization in the organic light emitting element, the material may have a high glass transition point. Examples of low-molecular-weight and high-molecular-weight materials having hole injection/transport properties include triarylamine derivatives, arylcarbazole derivatives, phenylenediamine derivatives, stilbene derivatives, phthalocyanine derivatives, porphyrin derivatives, poly(vinylcarbazole), poly(thiophene), and other conductive polymers. Furthermore, the aforementioned hole injection/transport material may be used in an electron blocking layer. Alternatively, a mixture (PEDOT:PSS) of polyethylenedioxythiophene and polystyrene sulfonate generally used as hole injection materials for manufacturing by a coating method may be used. Specific examples of the compounds used as hole injection/transport materials include, but are not limited to, compounds described below.

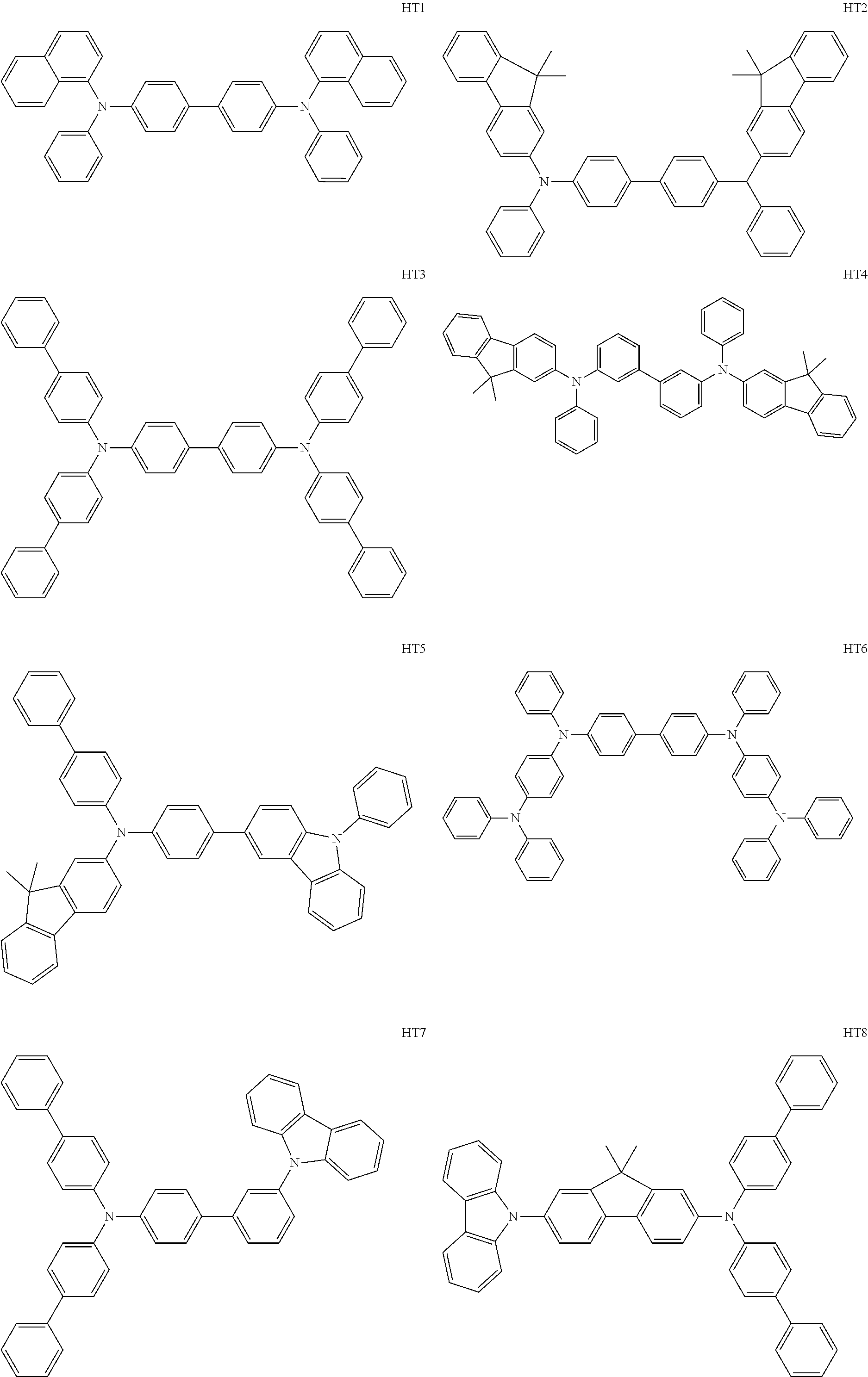

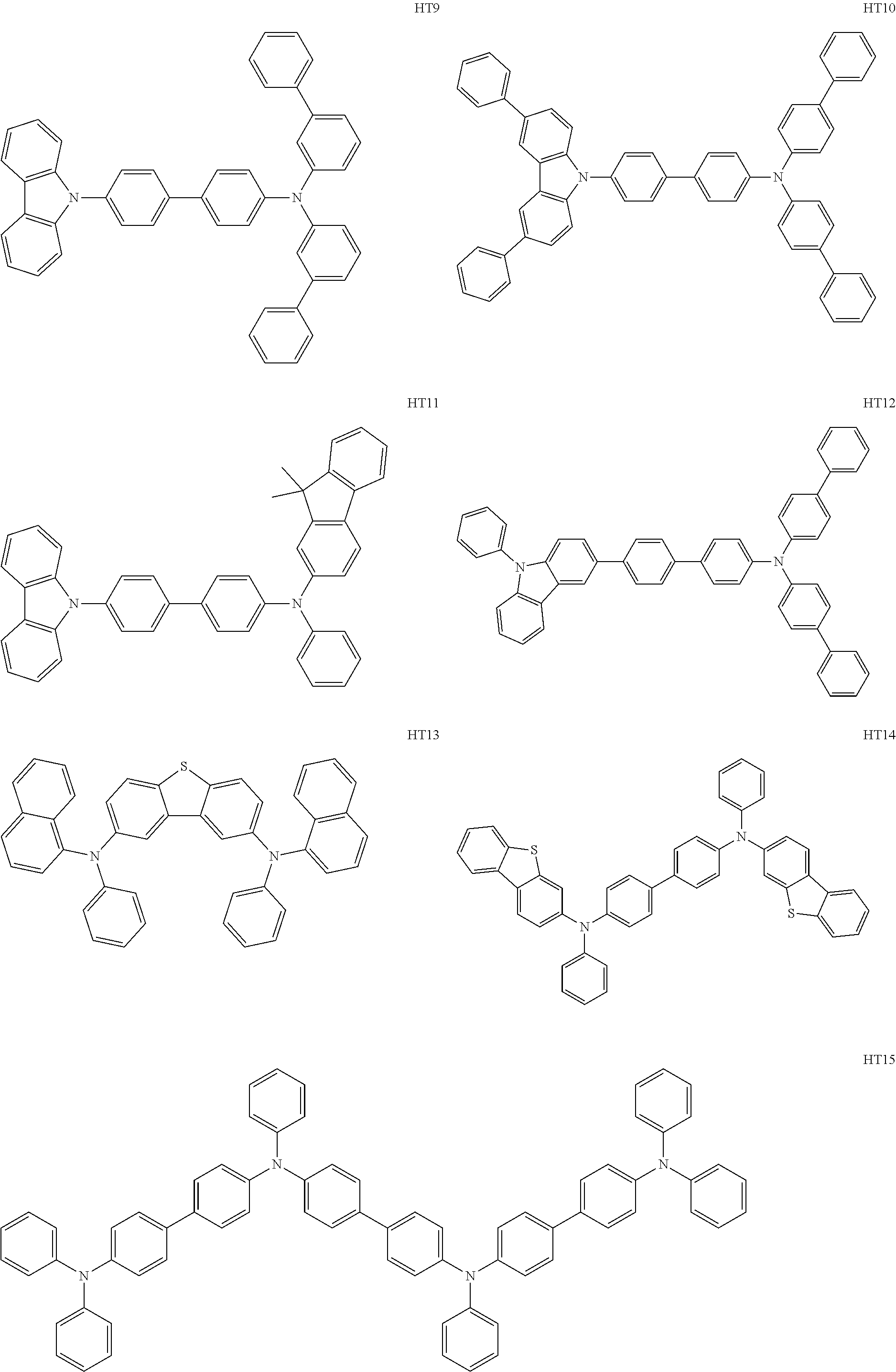

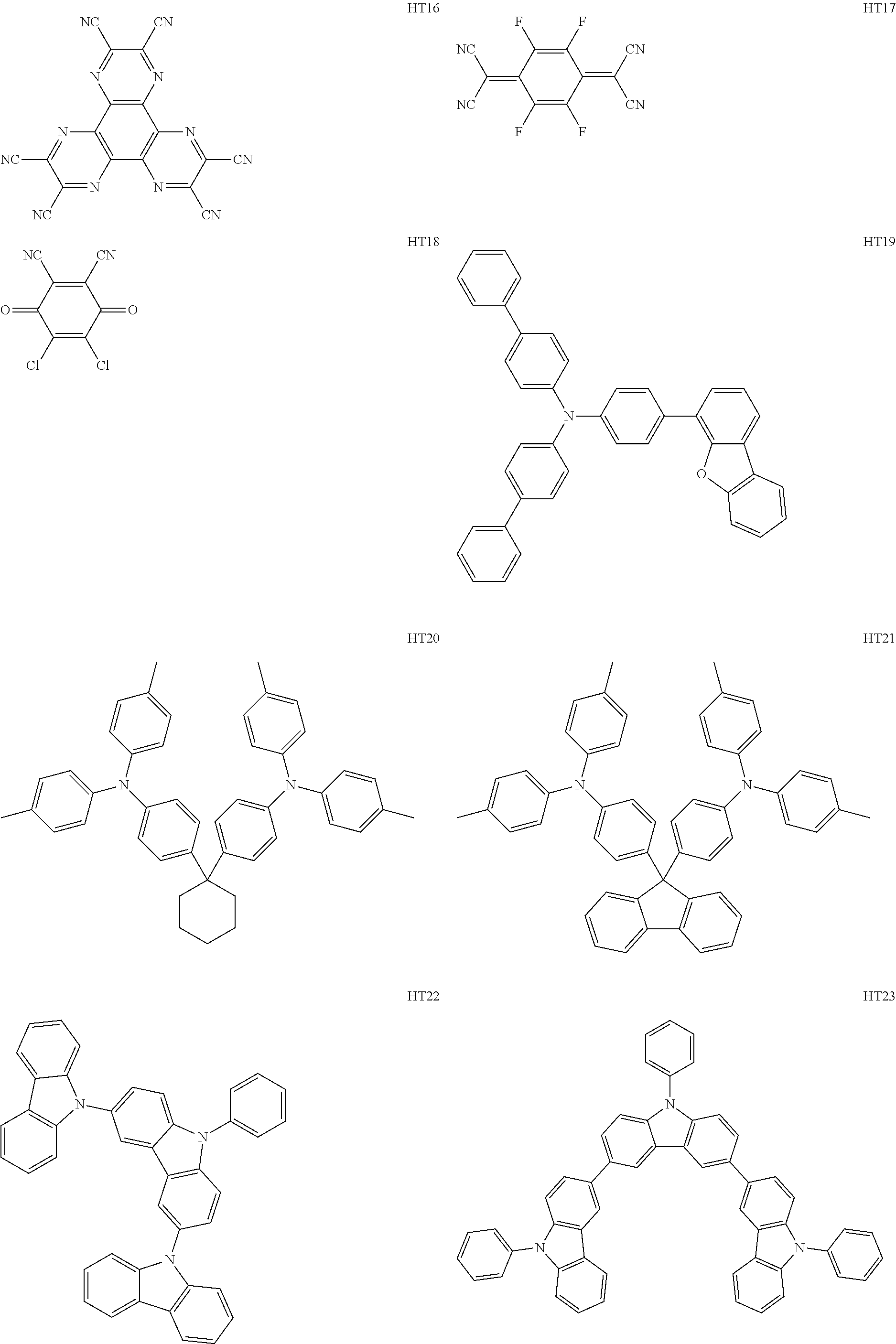

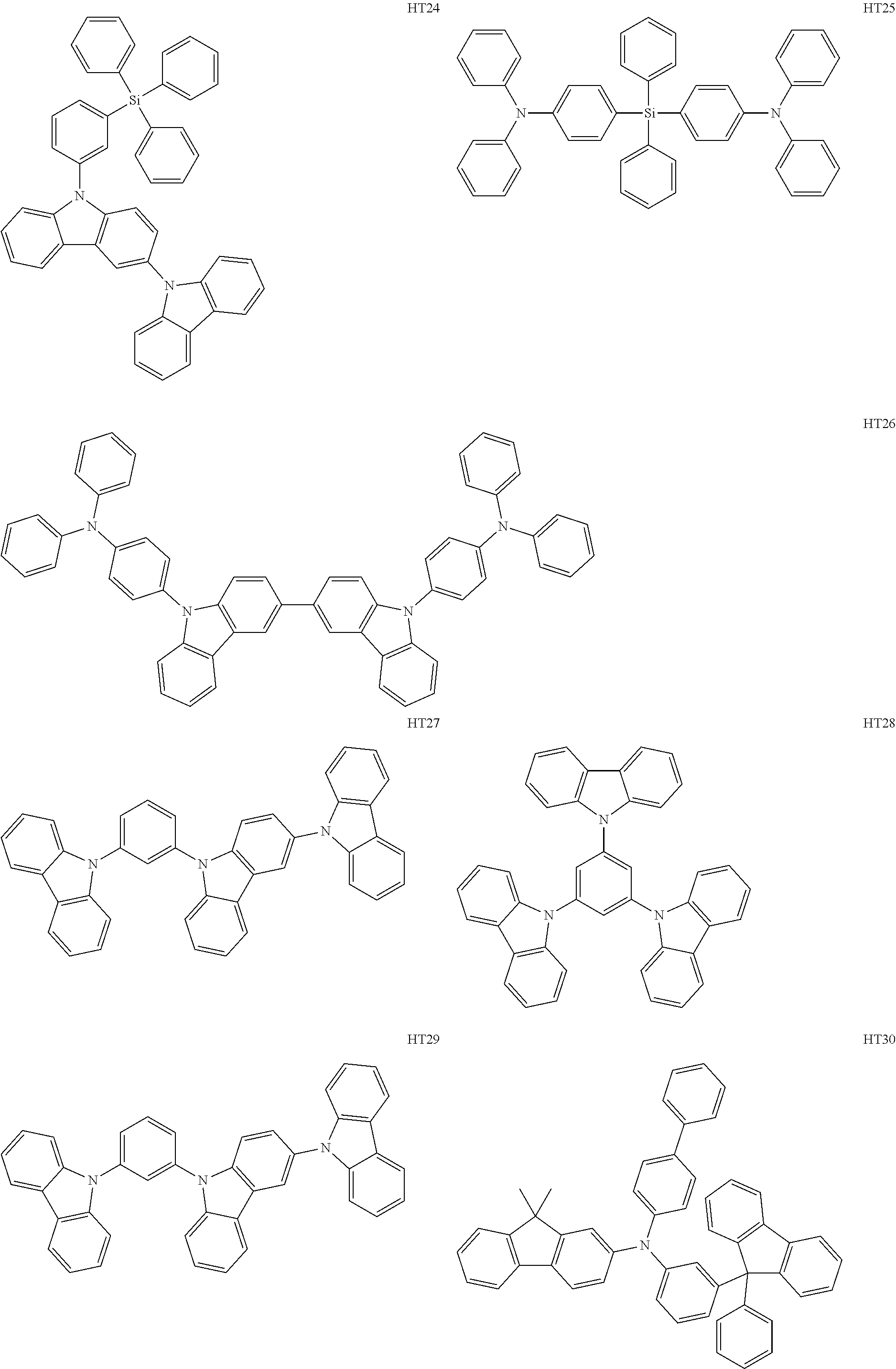

Among the hole transport materials listed, HT16 to HT18 can decrease the drive voltage when used in a layer in contact with the positive electrode. HT16 is widely used in organic light emitting elements.

HT2 to HT7, HT10, HT12, and HT22 to HT28 may be used in an organic compound layer adjacent to HT16. Hole transport polymer compounds such as polyphenylene vinylene (PPV), polyfluorene (PF), polyvinyl carbazole (PVK), and derivatives thereof may also be used. Alternatively, for example, insulator layers of inorganic materials such as SiO₂ and SiN, and organic silicon polymers such as siloxane can also be used. In addition, multiple materials may be used in one organic compound layer.

Examples of the light emitting materials mainly involved in the light emitting function include donor-acceptor organic compounds, boron-containing complexes, indocarbazole-fused compounds, fused compounds (for example, fluorene derivatives, naphthalene derivatives, pyrene derivatives, perylene derivatives, tetracene derivatives, anthracene derivatives, and rubrene), quinacridone derivatives, coumarin derivatives, stilbene derivatives, organoaluminum complexes such as tris(8-quinolinolato)aluminum, iridium complexes, platinum complexes, rhenium complexes, copper complexes, europium complexes, ruthenium complexes, and polymer derivatives such as poly(phenylenevinylene) derivatives, poly(fluorene) derivatives, and poly(phenylene) derivatives. Furthermore, when a light emitting layer is prepared by a coating method, a polymer compound having a light emitting property is mainly used. This is because a polymer compound has high amorphousness and is more resistant to crystallization than low-molecular-weight compounds. Specific examples of the material used include polyphenylene vinylene (PPV), polyfluorene (PF), polyvinyl carbazole (PVK), and derivatives thereof. Specific examples of the compounds used as the light emitting material include, but are not limited to, compounds described below.

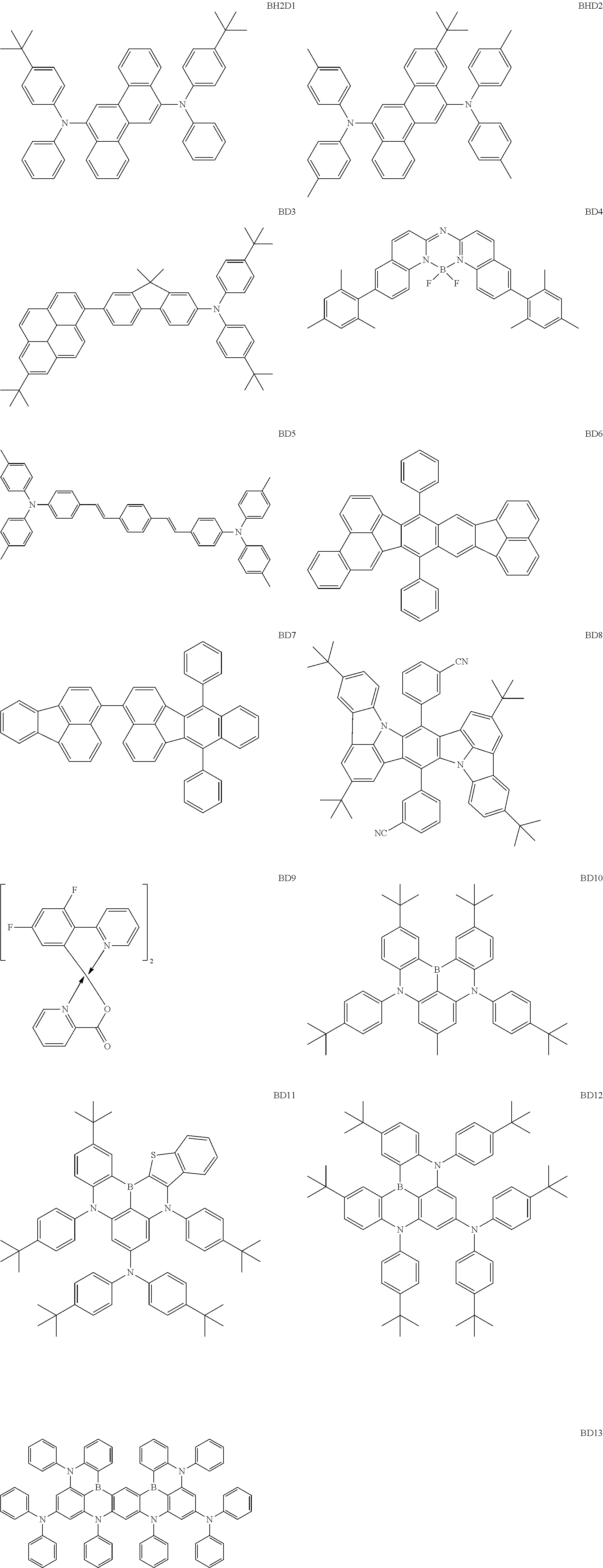

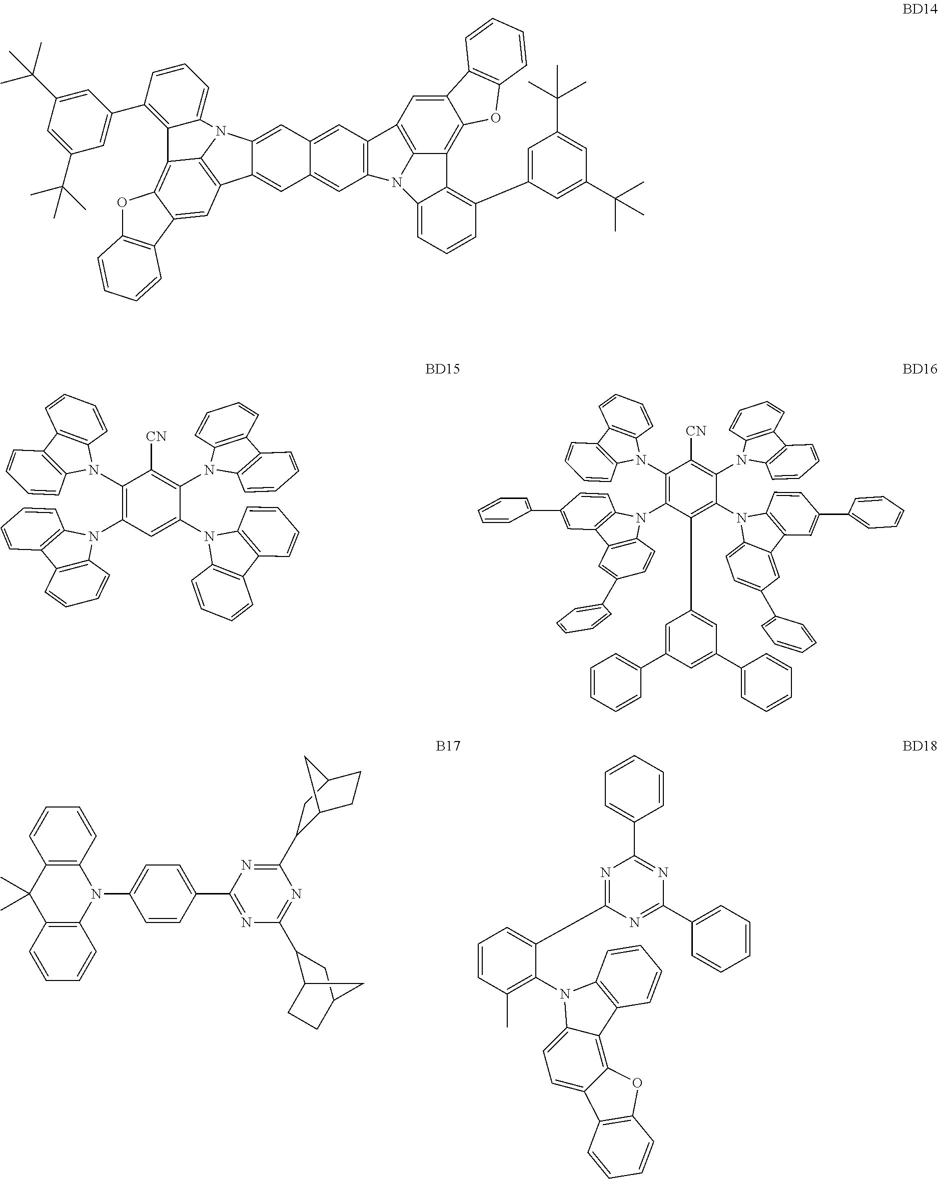

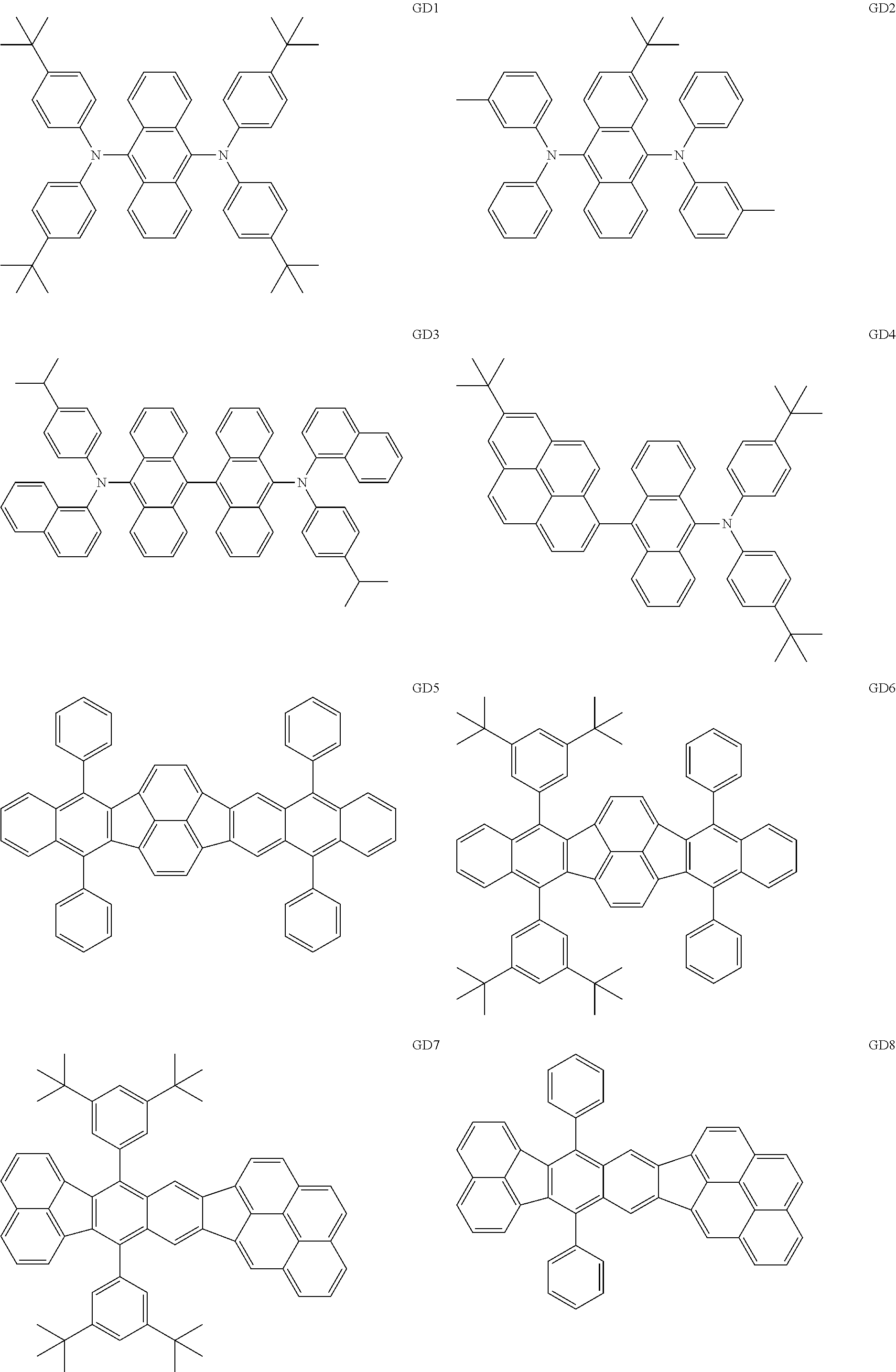

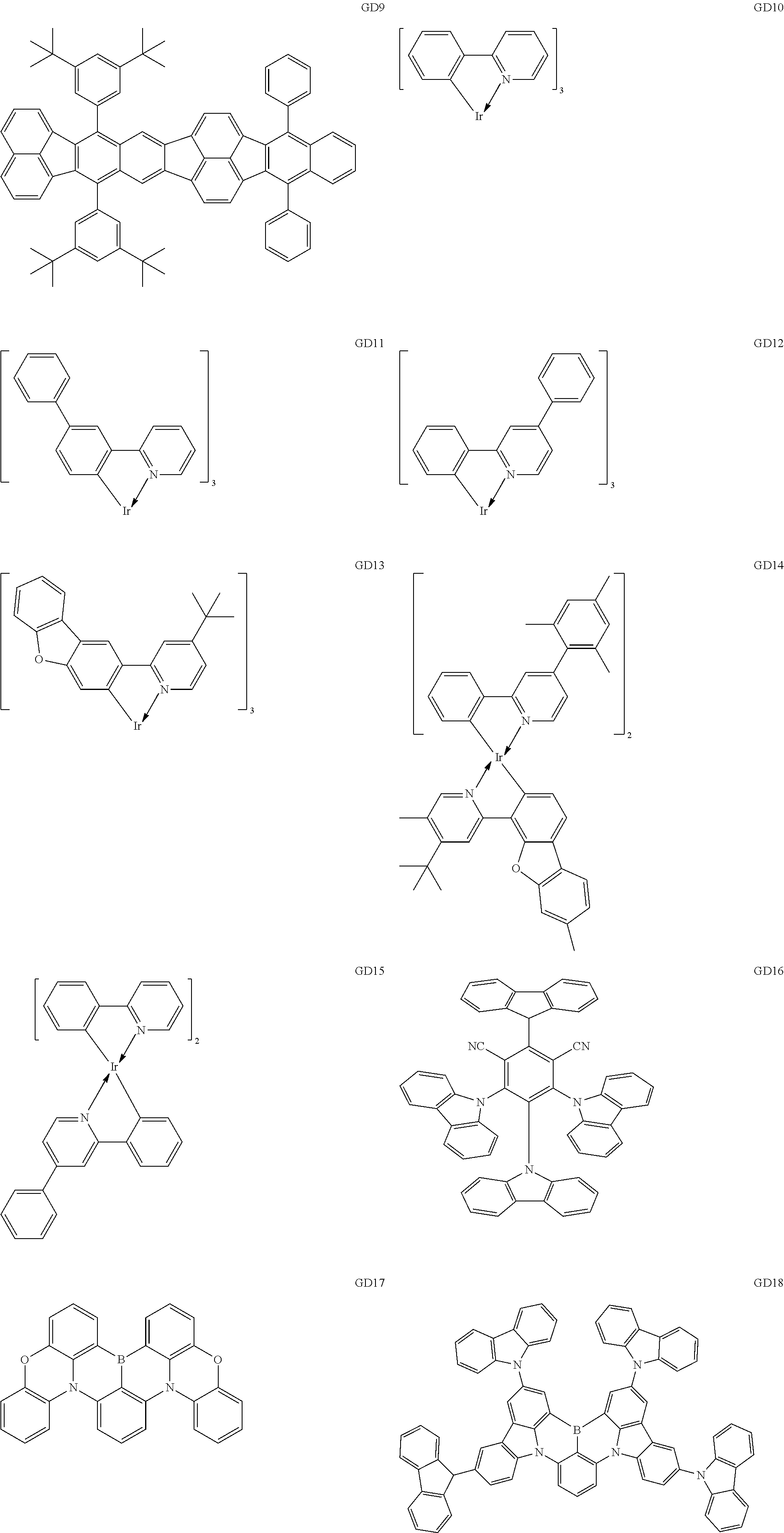

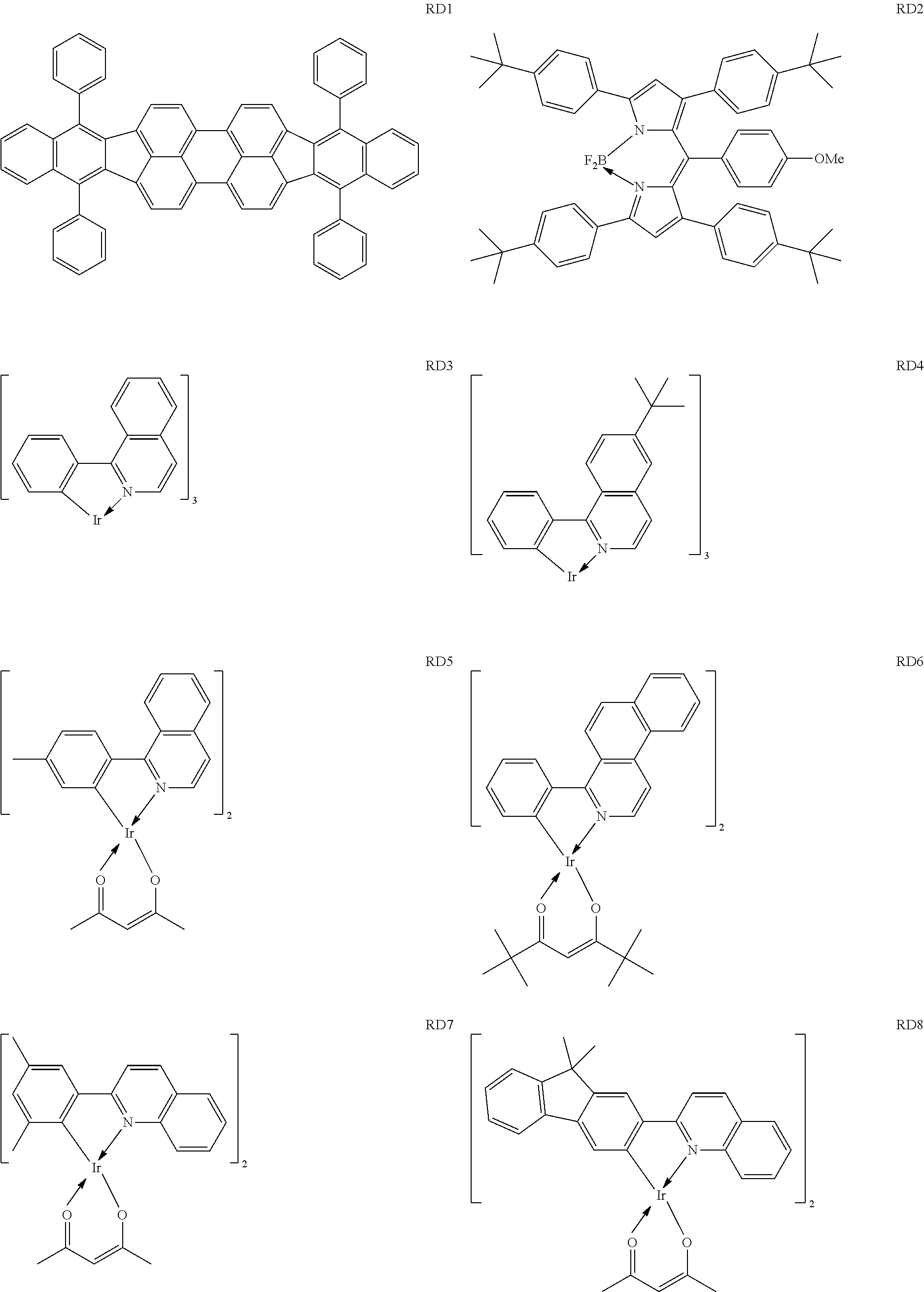

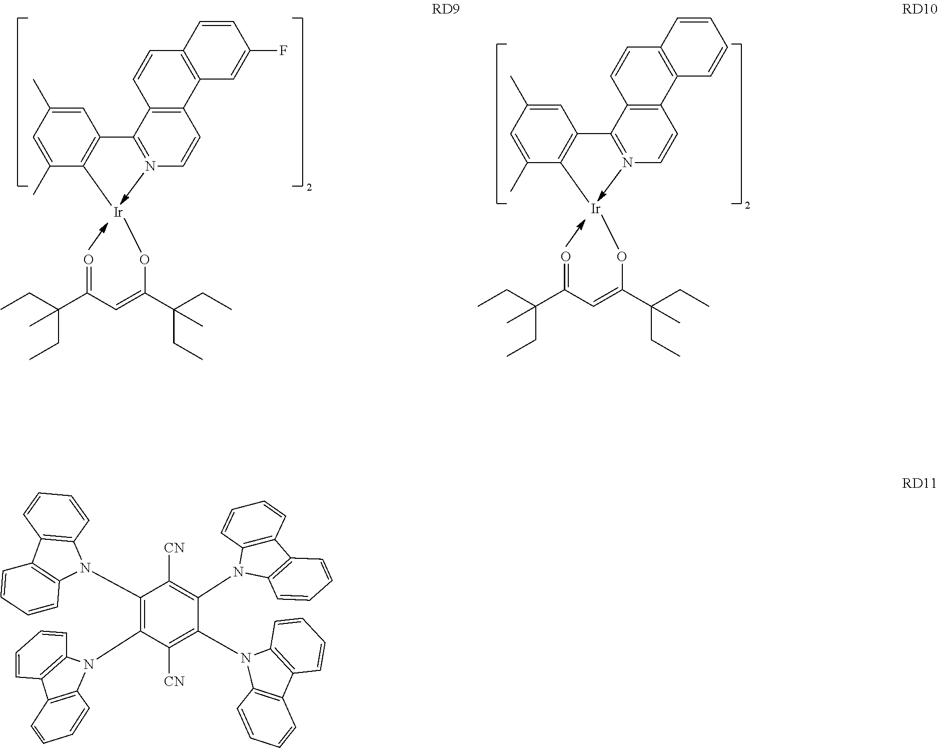

Specific examples of the compounds used as the light emitting layer host or the light emitting assist material contained in the light emitting layer include, but are not limited to, compounds described below.

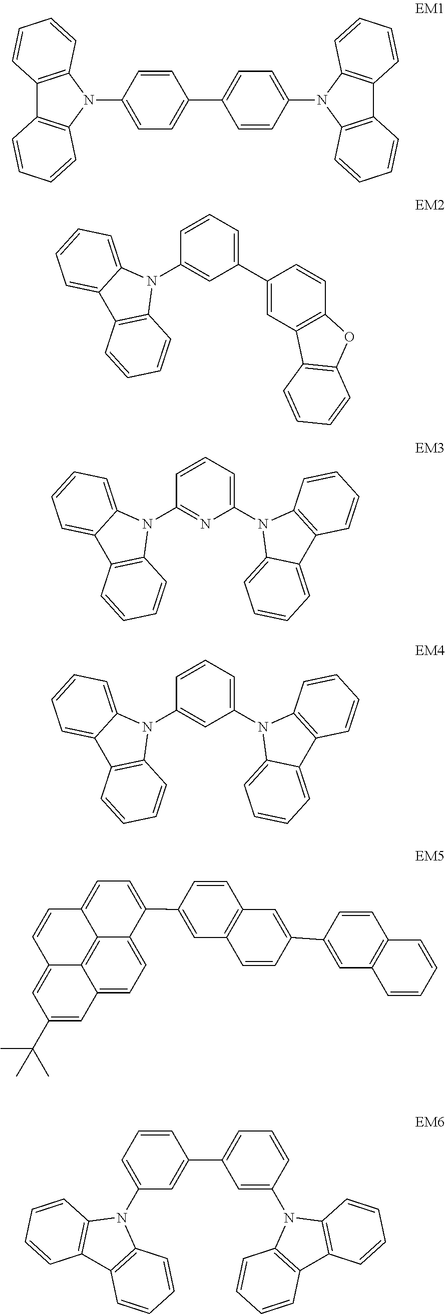

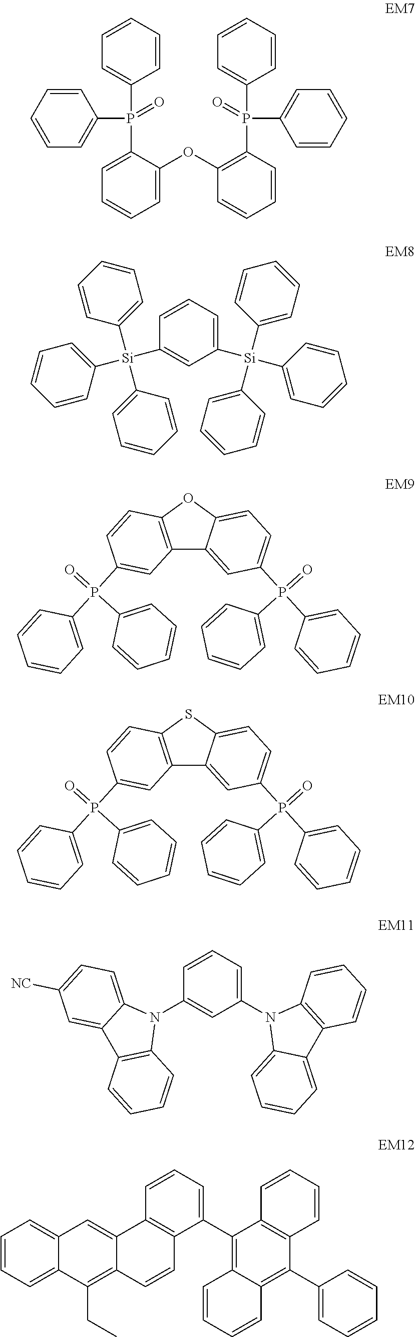

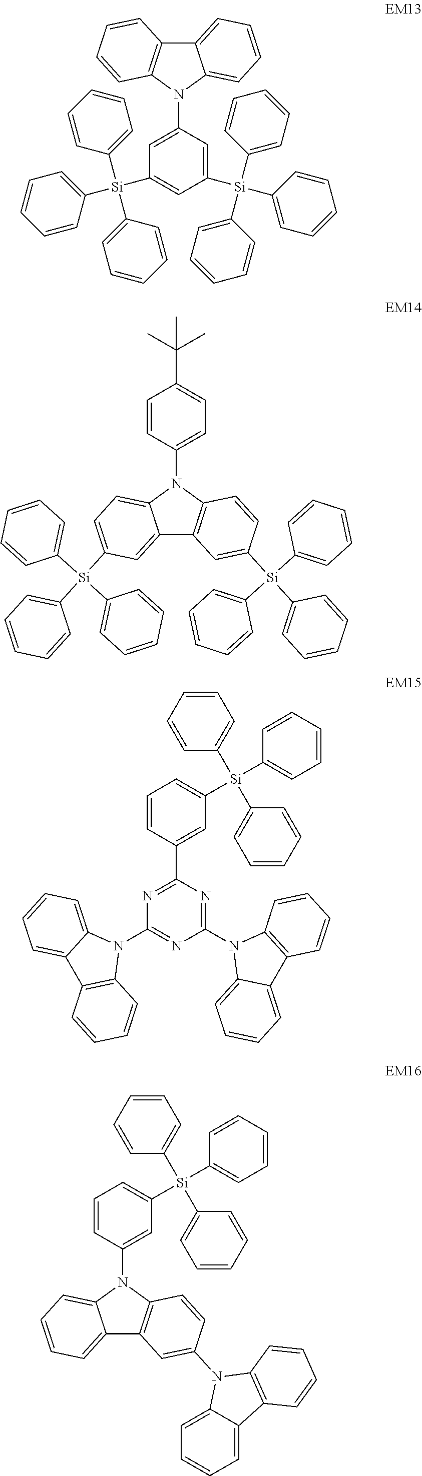

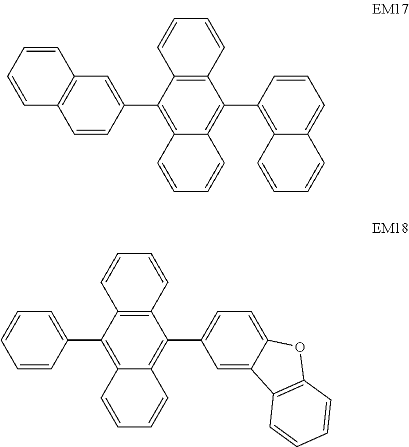

When the host material is used in combination with a delayed fluorescent material or a phosphorescent material, the triplet of the host material may be higher than the triplet of the delayed fluorescent material. Examples of the host material include, but are not limited to, EM2 to EM4, EM7 to EM11, and EM14 to EM16.

The electron transport material can be freely selected from those compounds which can transport electrons injected from the negative electrode to the light emitting layer and is selected by considering the balance with the hole mobility of the hole transport material, etc. Examples of the material having the electron transport property include oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused ring compounds (for example, fluorene derivatives, naphthalene derivatives, chrysene derivatives, and anthracene derivatives). Furthermore, the aforementioned electron transport materials may be used in a hole blocking layer.

Specific examples of the compounds used as the electron transport material include, but are not limited to, compounds described below.

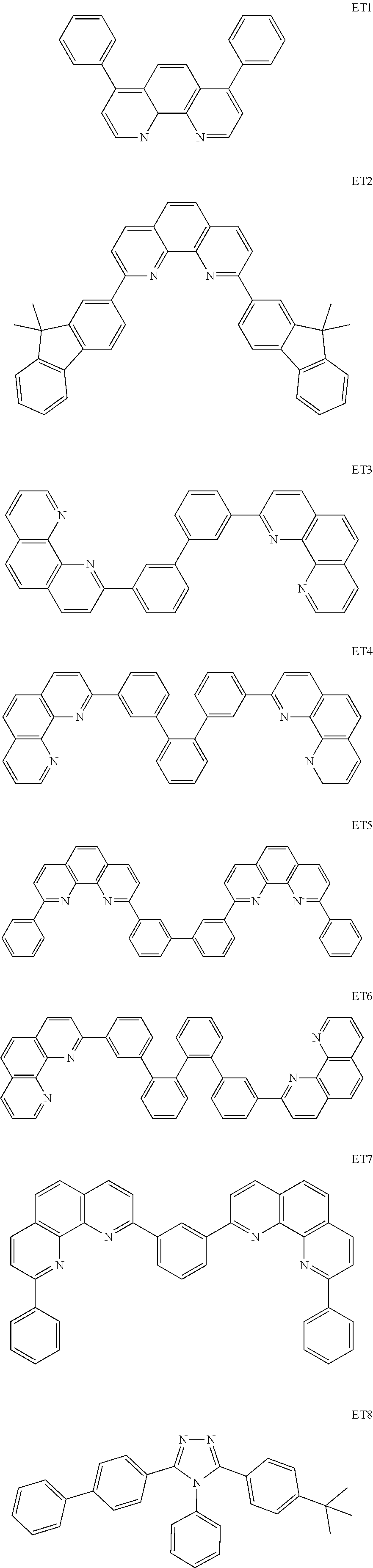

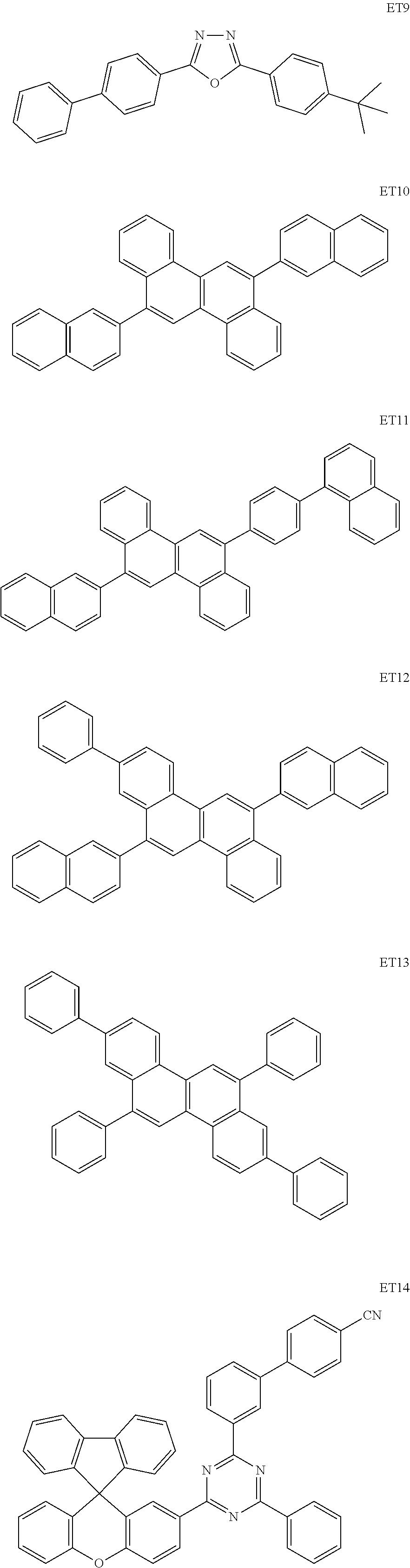

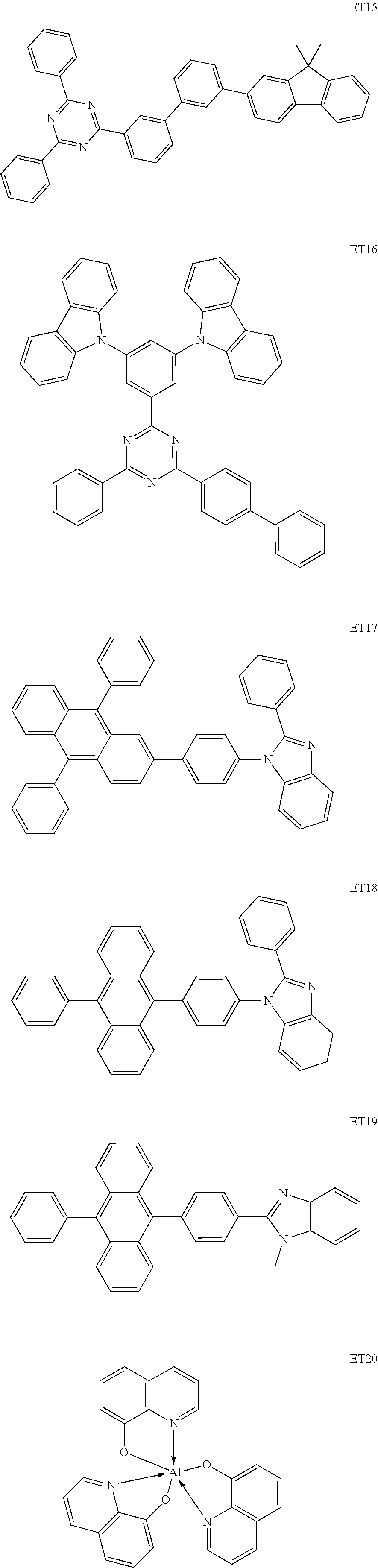

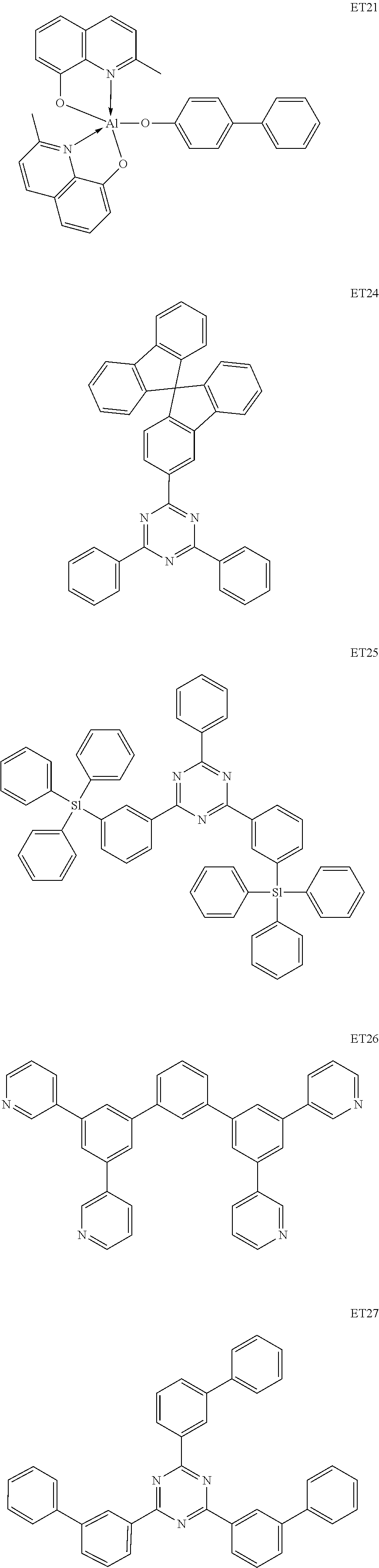

The electron injection material can be freely selected from those compounds which can facilitate electron injection from the negative electrode, and is selected by considering the balance with the hole injectability etc. The organic compound may include an n-type dopant or a reducing dopant. Examples of such an organic compound include alkali metal-containing compounds such as lithium fluoride, lithium complexes such as lithium quinolinol, benzimidazolidene derivatives, imidazolidene derivatives, fulvalene derivatives, and acridine derivatives. These compounds may be used in combination with the aforementioned electron transport materials.

### Structure of Organic Light Emitting Element

The organic light emitting element is prepared by sequentially forming an insulating layer, a first electrode, an organic compound layer, and a second electrode on a substrate. A protection layer, a color filter, a microlens, etc., may be formed on the second electrode. When a color filter is to be provided, a planarization layer may be formed between the color filter and the protection layer. The planarization layer can be made of an acrylic resin or the like. The same applies to the case in which a planarization layer is formed between a color filter and a microlens.

### Substrate

Examples of the substrate include quartz, glass, silicon wafers, resin, and metal. Switching elements such as transistors and wires may be disposed on the substrate, and an insulating layer may be disposed thereon. The material for the insulating layer may be any as long as contact holes can be formed therein to enable formation of wires that connect to the first electrode and as long as insulation between unconnected wires can be secured. Examples of such materials include resins such as polyimide, silicon oxide, and silicon nitride.

### Electrodes

A pair of electrodes can be used as the electrodes. A pair of electrodes include a positive electrode and a negative electrode. When an electric field is applied in a direction in which the organic light emitting element emits light, the electrode with a higher potential is the positive electrode, and the other electrode is the negative electrode. This can also be rephrased as that the electrode that supplies holes to the light emitting layer is the positive electrode, and the electrode that supplies electrons is the negative electrode.

The material constituting the positive electrode may have a work function as large as possible. Examples of such a material that can be used include single metals such as gold, platinum, silver, copper, nickel, palladium, cobalt, selenium, vanadium, and tungsten, mixtures thereof, and alloys thereof, and metal oxides such as tin oxide, zinc oxide, indium oxide, indium tin oxide (ITO), and indium zinc oxide. In addition, conductive polymers such as polyaniline, polypyrrole, and polythiophene can be used.

These electrode substances may be used alone or in combination of two or more. The positive electrode may be constituted by a single layer or multiple layers.

For use as a reflection electrode, chromium, aluminum, silver, titanium, tungsten, molybdenum, an alloy thereof, or a layered product thereof can be used. The aforementioned materials can be used to form a reflection film that does not function as an electrode. Moreover, for use as a transparent electrode, an oxide transparent conductive layer such as indium tin oxide (ITO) or indium zinc oxide can be used, but this is not limiting.

The electrode can be formed by photolithography.

In contrast, the material constituting the negative electrode can have a small work function. Examples of such a material include alkali metals such as lithium, alkaline earth metals such as calcium, single metals such as aluminum, titanium, manganese, silver, lead, and chromium, and mixtures thereof. In addition, alloys of these single metals can also be used. For example, magnesium-silver, aluminum-lithium, aluminum-magnesium, silver-copper, and zinc-silver can be used. Metal oxides such as indium tin oxide (ITO) can also be used. These electrode substances may be used alone or in combination of two or more. The negative electrode may have a single layer structure or a multilayer structure. In particular, silver can be used, or, in order to decrease aggregation of silver, a silver alloy can be used. The alloying ratio may be any as long as aggregation of silver can be decreased. For example, the ratio of silver to other metals may be 1:1, 3:1, or the like.

No limitation is imposed as the type of element, for example, an oxide conductive layer such as ITO can be used as the negative electrode to configure a top emission element, or a reflection electrode such as aluminum (Al) can be used as the negative electrode to configure a bottom emission element. The method for forming the negative electrode is not particularly limited; for example, a DC or AC sputtering method or the like may be employed to achieve good film coverage and ease of achieving lower resistance.

### Organic Compound Layer.

The organic compound layer may be constituted by a single layer or multiple layers. When the organic compound layer includes multiple layers, such layers may be referred to as a hole injection layer, a hole transport layer, an electron blocking layer, a light emitting layer, a hole blocking layer, an electron transport layer, and an electron injection layer according to the functions thereof. The organic compound layer is mainly composed of an organic compound but may contain inorganic atoms and inorganic compounds. For example, copper, lithium, magnesium, aluminum, iridium, platinum, molybdenum, zinc, and the like may be contained. The organic compound layer may be disposed between the first electrode and the second electrode and may be in contact with the first electrode and the second electrode.

The organic compound layer (hole injection layer, hole transport layer, electron blocking layer, light emitting layer, hole blocking layer, electron transport layer, electron injection layer, etc.) that constitutes the organic light emitting element according to an embodiment of the present disclosure is formed by the method described below.

The organic compound layer constituting the organic light emitting element according to an embodiment of the present disclosure can be formed by a dry process such as a vacuum deposition method, an ionized deposition method, sputtering, or plasma. Instead of the dry process, a wet process that involves dissolving the materials in an appropriate solvent and forming layers by a known coating method using the resulting solution (for example, spin coating, casting, microgravure coating, gravure coating, bar coating, roll coating, wire bar coating, dip coating, spray coating, screen printing, flexographic printing, offset printing, inkjet printing, capillary coating, and nozzle coating) may be employed. In particular, the vacuum deposition method, the ionized deposition method, the inkjet printing method, the nozzle coating method, and the like may be used in producing large-area organic light emitting elements.

When a compound having high solubility in organic solvents is used among the organic compounds of the present embodiment to form a light emitting layer, the coating method may be employed. Examples of the coating method include a spin coating method, a slit coater method, a printing method, an inkjet method, a dispensing method, and a spraying method. Alternatively, the light emitting layer may be formed by the vacuum deposition method.

A layer formed by a vacuum deposition method, a solution coating method, or the like is resistant to crystallization or the like and has excellent stability over time. When the coating method is employed to form a film, an appropriate binder resin may be used in combination to form the film.

Examples of the binder resin include, but are not limited to, polyvinyl carbazole resin, polycarbonate resin, polyester resin, ABS resin, acrylic resin, polyimide resin, phenolic resin, epoxy resin, silicone resin, and urea resin.

These binder resins may be used alone as a homopolymer or in combination as a mixture of two or more to form a copolymer. If necessary, additives such as known plasticizers, oxidation inhibitors, and ultraviolet absorbents may be used in combination.

The thickness of each of the layers in the organic light emitting element is typically 1 nm or more and 10 μm or less. In particular, the thickness of the light emitting layer of the organic compound layer may be 10 nm or more and 100 nm or less to obtain effective light emitting properties.

### Protection Layer

A protection layer may be formed on the second electrode. For example, by bonding a hygroscopic agent-laden glass onto the second electrode, penetration of water and the like into the organic compound layer can be reduced, and the incidence of display failures can be decreased. In another embodiment, a passivation film such as silicon nitride may be disposed on the second electrode so as to reduce penetration of water and the like into the organic compound layer. For example, a protection layer may be formed by forming a 2 μm-thick silicon nitride film on the second electrode by a CVD method after the second electrode is formed and then transported to another chamber without breaking the vacuum. Alternatively, after a film is formed by a CVD method, a protection layer may be formed by an atomic layer deposition (ALD) method. The material for the film formed by the ALD method is not particularly limited and may be silicon nitride, silicon oxide, or aluminum oxide, for example. Silicon nitride may be further formed by a CVD method on the film formed by the ALD method. The film formed by the ALD method may be thinner than the film formed by the CVD method. Specifically, the thickness may be 50% or less or even 10% or less.

### Color Filter

A color filter may be formed on the protection layer. For example, a color filter that takes into account the size of the organic light emitting element may be formed on a separate substrate, and this substrate may be bonded with the substrate on which the organic light emitting element has been formed, or a color filter may be formed by photolithographic patterning on the aforementioned protection layer. The color filter may be composed of a polymer.

### Planarization Layer

A planarization layer may be disposed between the color filter and the protection layer. The planarization layer is formed for the purpose of decreasing unevenness of the underlying layer. The planarization layer may also be referred to as a material resin layer without limiting the purpose. The planarization layer may be composed of an organic compound and may have a low molecular weight or a high molecular weight; however, the planarization layer can have a high molecular weight.

Planarization layers may be disposed above and under the color filter, and in such a case, the materials constituting the planarization layers may be the same or different. Specific examples of the material include polyvinyl carbazole resin, polycarbonate resin, polyester resin, ABS resin, acrylic resin, polyimide resin, phenolic resin, epoxy resin, silicone resin, and urea resin.

### Microlens

The organic light emitting element may have an optical member, such as a microlens, on the light incident side thereof. The microlens can be composed of an acrylic resin, an epoxy resin, or the like. The microlens may be provided for the purposes of increasing the amount of light emerging from the organic light emitting element and controlling the direction in which the light emerges. The microlens may have a hemispherical shape. When the microlens has a hemispherical shape, there are tangent lines that contact the hemisphere, among which there is a tangent line parallel to the insulating layer, and the point of contact between this tangent line and the hemisphere is the apex of the microlens. The apex of the microlens can also be determined in the same manner from any cross-sectional view thereof. In other words, among the tangent lines that contact the semicircle of the microlens in a cross-sectional view, there is a tangent line parallel to the insulating layer, and the point of contact between this tangent line and the semicircle is the apex of the microlens.

Furthermore, the midpoint of the microlens can be defined. In a cross section of the microlens, a line segment extending from a point where the shape or an arc ends to another point where the shape of the arc ends is hypothetically drawn, and the midpoint of that line segment can be called the midpoint of the microlens. The cross section used to determine the apex and the midpoint may be a cross section perpendicular to the insulating layer.

### Opposing Substrate

An opposing substrate may be provided on the planarization layer. The opposing substrate is called an opposing substrate since the opposing substrate is disposed at a position opposing the aforementioned substrate. The material constituting the opposing substrate may be the same as the aforementioned substrate. The opposing substrate may be a second substrate provided that the aforementioned substrate is a first substrate.

### Pixel Circuit

An organic light emitting apparatus that includes an organic light emitting element may include a pixel circuit connected to the organic light emitting element. The pixel circuit may be of an active matrix type that independently controls light emission of a first light emitting element and a second light emitting element. The active matrix circuit may be voltage-programmed or current-programmed. The drive circuit has a pixel circuit for each pixel. The pixel circuit may include a light emitting element, a transistor that controls the emission luminance of the light emitting element, a transistor that controls the light emission timing, a capacitor that retains the gate voltage of the transistor that controls the emission luminance, and a transistor for establishing the connection to GND without passing through the light emitting element.

The light emitting apparatus has a display region and a peripheral region around the display region. The display region includes pixel circuits, and the peripheral region includes display control circuits. The mobility of the transistor constituting the pixel circuit may be smaller than the mobility of the transistor constituting the display control circuit.

The gradient of the current-voltage characteristic of the transistor constituting the pixel circuit may be smaller than the gradient of the current-voltage characteristic of the transistor constituting the display control circuit. The gradient of the current-voltage characteristic can be measured by what is commonly known as Vg-Ig characteristics. The transistor constituting the pixel circuit is a transistor connected to alight emitting element, such as the first light emitting element.

### Pixels

The organic light emitting apparatus that includes the organic light emitting element may include multiple pixels. Each pixel has subpixels that emit light of colors different from one another. The subpixels may emit light of RGB colors, for example.

The pixel has a region also known as a pixel aperture that emits light. The pixel aperture may be 15 μm or less and may be 5 μm or more. More specifically, the pixel aperture may be 11 μm, 9.5 μm, 7.4 μm, 6.4 μm, etc. The distance between the subpixels can be 10 μm or less, specifically, 8 μm, 7.4 μm, or 6.4 am.

The pixels may be arranged into any known pixel matrix in a plan view. Examples of the pixel matrix include a stripe matrix, a delta matrix, a pentile matrix, and a bayer matrix. The shape of the subpixels in a plan view may be any known shape. Examples thereof include rectangular shapes such as an oblong shape or a rhombus shape, and hexagonal shapes. Naturally, the shape does not have to be exact, and any shape close to an oblong shape is considered an oblong shape. The shape of the subpixels and the pixel arrangement may be used in combination.

### Usage of Organic Light Emitting Element

The organic light emitting element according to the present embodiment can be used as a member that constitutes a display apparatus or a lighting apparatus. Other examples of the usage include an exposure light source of an electrophotographic image forming apparatus, a backlight of a liquid crystal display apparatus, and a light emitting apparatus that has a color filter on a white light source.

The display apparatus may be an information processing apparatus that includes an image input section through which image information is input from an area CCD, a linear CCD, a memory card, or the like, and an information processing section processing the input information, and that displays the input image on a display section. The display apparatus may include multiple pixels, and at least one of the pixels may include the organic light emitting element of the present embodiment and an active element, such as a transistor, connected to the organic light emitting element. Here, the substrate may be a semiconductor substrate such as silicon, and the transistor may be a MOSFET formed on the substrate. The image display apparatus includes an input section for inputting image information and a display section for outputting an image, and the display section includes the display apparatus of the present embodiment.

Furthermore, a display section of an imaging apparatus or an inkjet printer may have a touch panel function. The driving system of the touch panel function may be infrared radiation, electrostatic capacitance, resistance film, electromagnetic induction, or any other system. The display apparatus may be used in a display section of a multifunctional printer.

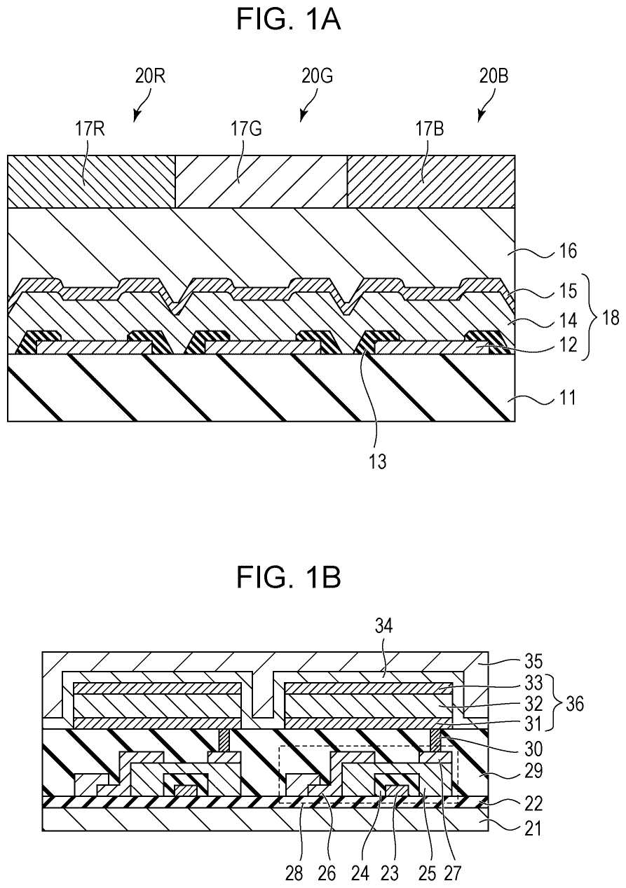

Next, a display apparatus according to the present embodiment is described with reference to the drawings. FIGS. 1A and 1B are schematic cross sectional views illustrating examples of a display apparatus that includes organic light emitting elements and transistors connected to the organic light emitting elements. The transistors are one example of the active element. The transistors may be thin film transistors (TFTs).

FIG. 1A is a schematic cross-sectional view of one example of a pixel which is a constitutional feature of the display apparatus of the present embodiment. The pixel has subpixels 20. The subpixels are classified as 20R, 20G, and 20B according to their emission color. The emission color may be distinguished by the wavelength of the light emitted from the light emitting layer, or a color filter or the like may be used to selectively transmit or change the color of the light emitted from the subpixels. Each of the subpixels 20 has a first electrode 12 that serves as a reflection electrode disposed on an interlayer insulating layer 11, an insulating layer 13 that covers edges of the first electrode 12, an organic compound layer 14 that covers the first electrode 12 and the insulating layer 13, a second electrode 15 which is a transparent electrode, a protection layer 16, and a color filter 17.

A transistor and a capacitor element may be disposed under or inside the interlayer insulating layer 11. The transistor and the first electrode 12 may be electrically connected to each other via a contact hole or the like not illustrated in the drawing.

The insulating layer 13 is also referred to as a bank or a pixel isolation film. The insulating layer 13 covers the edges of the first electrode 12 and surrounds the first electrode 12. The part where the insulating layer 13 is absent is in contact with the organic compound layer 14 and functions as a light emitting region.

The second electrode 15 may be a transparent electrode, a reflection electrode, or a semi-transmissive electrode.

The protection layer 16 reduces penetration of moisture into the organic compound layer 14. Although the protection layer 16 is depicted as one layer in the drawing, the protection layer 16 may include multiple layers. An inorganic compound layer and an organic compound layer may be provided for each layer.

The color filters 17 are classified as 17R, 17G, and 17B according to their colors. The color filters 17 may be formed on a planarizing film not illustrated in the drawing. A resin protection layer not illustrated in the drawing may be disposed on the color filters 17. The color filters 17 may be formed on the protection layer 16. Alternatively, the color filters 17 may be formed on an opposing substrate such as a glass substrate and then bonded with the substrate.

A display apparatus illustrated in FIG. 1B includes an organic light emitting element 36 and a TFT 28 which is one example of the transistor. An insulating layer 22 is disposed on a substrate 21 such as glass or silicon. Active elements such as a TFT 28 are disposed on the insulating layer 22, and a gate electrode 23, a gate insulating film 24, and a semiconductor layer 25 of the active element are disposed on the insulating layer 22. The TFT 28 has a drain electrode 26 and a source electrode 27. An insulating layer 29 is disposed on the TFT 28. A positive electrode 31 constituting the organic light emitting element 36 and the source electrode 27 are connected to each other via a contact hole 30 formed in the insulating layer 29.

Here, the type of electrical connection between the electrodes (positive electrode 31 and negative electrode 33) included in the organic light emitting element 36 and the electrodes (source electrode 27 and drain electrode 26) included in the TFT 28 is not limited to the one illustrated in FIG. 1B. In other words, the type of electrical connection may be any as long as one of the positive electrode 31 and the negative electrode 33 is electrically connected to one of the source electrode 27 and the drain electrode 26 of the TFT 28.

In the display apparatus illustrated in FIG. 1B, the organic compound layer 32 is depicted as one layer, but the organic compound layer 32 may include multiple layers. A first protection layer 34 and a second protection layer 35 are disposed on the negative electrode 33 to reduce deterioration of the organic light emitting element 36.

The display apparatus illustrated in FIG. 1B uses a transistor as a switching element, but may use a different switching element, such as an MIM element, instead.

The transistor used in the display apparatus illustrated in FIG. 1B is not limited to a thin film transistor having an active layer on an insulating surface of the substrate and may be a transistor that uses a single-crystal silicon wafer. Examples of the active layer include single crystal silicon, non-single-crystal silicon such as amorphous silicon or microcrystalline silicon, or a non-single-crystal oxide semiconductor such as indium zinc oxide or indium gallium zinc oxide. Note that a thin film transistor may also be referred to as a TFT element.

The transistor included in the display apparatus illustrated in FIG. 1B may be formed inside the substrate such as a Si substrate. Here, the phrase “formed inside the substrate” means that the transistor is produced by processing a substrate, such as a Si substrate, itself. In other words, “transistor inside the substrate” can also be considered as that the substrate and the transistor are integrated.

The emission luminance of the organic light emitting element of the present embodiment is controlled by the TFT, which is one example of the switching element, and multiple organic light emitting elements are disposed within the surface so as to display images through emission luminance of each organic light emitting element. Note that the switching element of the present embodiment is not limited to a TFT and may be a transistor made of a low-temperature polysilicon or an active matrix driver formed on a substrate such as a Si substrate. The phrase “on the substrate” can also mean “inside the substrate”. Whether a transistor is formed inside the substrate or whether a TFT is used is selected according to the size of the display section; for example, when the size of the display section is about 0.5 inches, organic light emitting elements may be formed on a Si substrate.

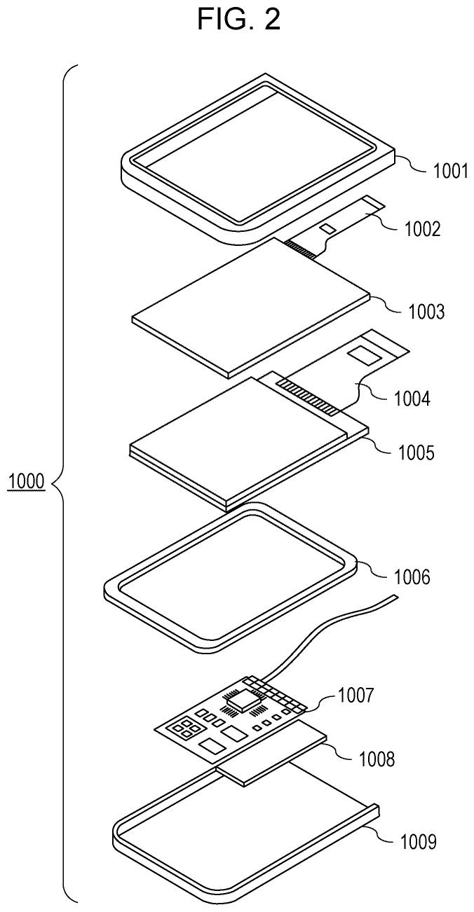

FIG. 2 is a schematic diagram of one example of a display apparatus according to one embodiment. A display apparatus 1000 may include an upper cover 1001 and a lower cover 1009, and a touch panel 1003, a display panel 1005, a frame 1006, a circuit substrate 1007, and a battery 1008 that are interposed between these covers. The touch panel 1003 and the display panel 1005 are respectively connected to flexible print circuits FPC 1002 and 1004. Transistors are printed on the circuit substrate 1007. When the display apparatus is not a portable appliance, the battery 1008 may be omitted, and even when the display apparatus is a portable appliance, the battery 1008 may be provided in a different place.

The display apparatus of the present embodiment may include red, green and blue color filters. The red, green and blue color filters may be arranged in a delta matrix.

The display apparatus of the present embodiment may be used in a display section of a portable terminal. Such a display apparatus may have both a display function and an operation function. Examples of the portable terminal include mobile phones such as smart phones, tablets, and head-mount displays.

The display apparatus of the present embodiment may be used in a display section of an imaging apparatus that includes an optical section having multiple lenses and an imaging element that receives light that has passed through the optical section. The imaging apparatus may include a display section that displays the information acquired by the imaging element. The display section may be exposed to the outside of the imaging apparatus or may be disposed in a finder. The imaging apparatus may be a digital camera or a digital camcorder.

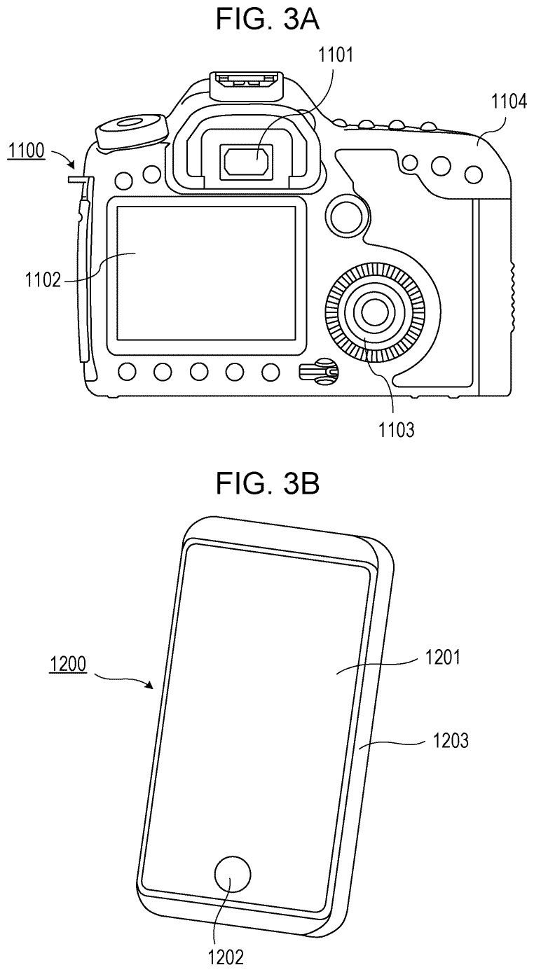

FIG. 3A is a schematic diagram of one example of an imaging apparatus according to an embodiment of the present disclosure. An imaging apparatus 1100 may include a view finder 1101, a rear display 1102, an operation section 1103, and a casing 1104. The view finder 1101 includes the display apparatus according to the present embodiment. The display apparatus may display not only the image to be captured but also the environment information, imaging instructions, etc. The environment information may include the intensity of external light, the direction of the external light, the speed in which a photographic subject moves, and the possibility of the photographic subject becoming shielded by a shielding material.

Since the timing for imaging is very short, it is better to display the information as quickly as possible. Accordingly, a display apparatus that uses the organic light emitting element of the present embodiment is used. This is because the organic light emitting element has high response speed. A display apparatus that uses an organic light emitting element has an advantage over liquid crystal display apparatuses for use in such devices required to achieve speedy display.

The imaging apparatus 1100 includes an optical section not illustrated in the drawing. The optical section has multiple lenses, and an image is focused on the imaging element housed in the casing 1104. The focal point can be adjusted by adjusting the relative positions of the multiple lenses. This operation can be automated. The imaging apparatus may also be referred to as a photoelectric conversion apparatus. The photoelectric conversion apparatus does not have to sequentially capture images and can employ, for example, an imaging method that involves detecting the difference from a previous image or constantly snipping an image from already recorded images.

FIG. 3B is a schematic diagram of one example of electronic equipment according to an embodiment. Electronic equipment 1200 includes a display section 1201, an operation section 1202, and a casing 1203. The casing 1203 may include a circuit, a print substrate having the circuit, a battery, and a communication section. The operation section 1202 may be a button or a touch panel-system sensitive section. The operation section 1202 may be a biometric section that performs, for example, unlocking through fingerprint recognition. The electronic equipment that includes the communication section can also be called communication equipment. The electronic equipment 1200 may be equipped with a lens and an imaging element to acquire a camera function. The image captured through the camera function is displayed on the display section 1201. Examples of the electronic equipment 1200 include smart phones and laptop computers.

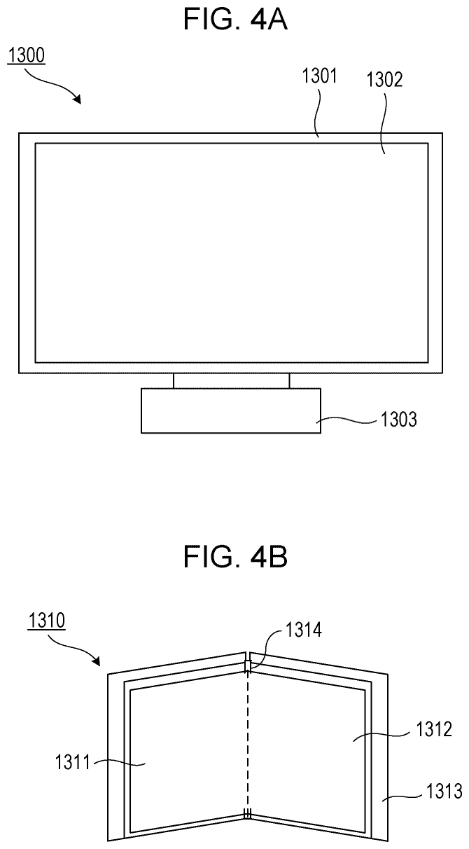

FIGS. 4A and 4B are schematic diagrams of some examples of the display apparatus according to one embodiment. FIG. 4A illustrates a display apparatus such as a television monitor or a PC monitor. A display apparatus 1300 includes a frame 1301 and a display section 1302. The light emitting element of the present embodiment is used in the display section 1302. The display apparatus 1300 also includes a base 1303 that supports the frame 1301 and the display section 1302. The base 1303 is not limited to the type illustrated in FIG. 4A. The lower edge of the frame 1301 may also serve as the base. The frame 1301 and the display section 1302 may be curved. The radius of curvature thereof may be 5000 mm or more and 6000 mm or less.

FIG. 4B is a schematic diagram of another display apparatus according to the present embodiment. A display apparatus 1310 illustrated in FIG. 4B is bendable, in other words, a foldable display apparatus. The display apparatus 1310 includes a first display section 1311, a second display section 1312, a casing 1313, and an inflection point 1314. The first display section 1311 and the second display section 1312 each include a light emitting element of the present embodiment. The first display section 1311 and the second display section 1312 may constitute one seamless display apparatus. The first display section 1311 and the second display section 1312 can be separated at the inflection point. The first display section 1311 and the second display section 1312 may display different images, or may together display one image.

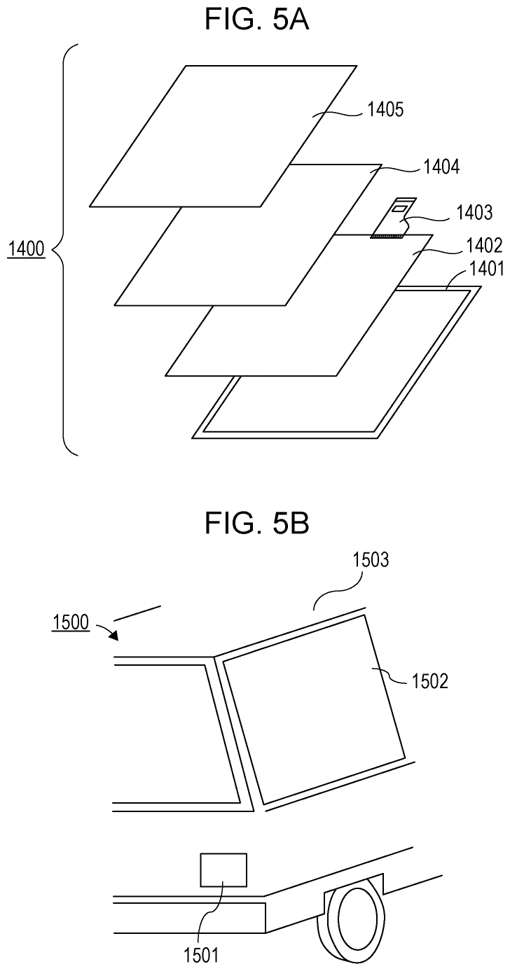

FIG. 5A is a schematic diagram of one example of a lighting apparatus according to the present embodiment. A lighting apparatus 1400 includes a casing 1401, a light source 1402, a circuit substrate 1403, and an optical filter 1404 and a light diffusing section 1405 that transmit light emitted from the light source 1402. The light source 1402 includes the organic light emitting element of the present embodiment. The optical filter 1404 may be a filter that improves the color rendering properties of the light source. The light diffusing section 1405 can effectively diffuse light from the light source, such as for lighting-up, and can deliver the light to a wide range. The optical filter 1404 and the light diffusing section 1405 may be disposed on the light emitting side of the lighting. If necessary, a cover may be provided in the outermost part.

The lighting apparatus is, for example, an apparatus that illuminates the room. The lighting apparatus may emit light that is white, neutral white, or any color from blue to red. The lighting apparatus may also include a light modulating circuit that modulates light and a light modulating circuit that modulates the emission colors. The lighting apparatus includes an organic light emitting element of the present embodiment and a power supply circuit connected to the organic light emitting element. The power supply circuit is a circuit that converts AC voltage into DC voltage. The lighting apparatus may also include an inverter circuit. White is a color that has a color temperature of 4200 K and neutral white is a color that has a color temperature of 5000 K. The lighting apparatus may include a color filter.

The lighting apparatus of the present embodiment may include a heat releasing section. The heat releasing section is a section that releases the heat inside the apparatus to the outside of the apparatus, and examples thereof include metals having a high specific heat, and liquid silicone.

FIG. 5B is a schematic diagram of an automobile which is one example of a moving body according to the present embodiment. The automobile includes a tail lamp, which is one example of a lighting section. An automobile 1500 may include a tail lamp 1501 and may light the tail lamp upon braking operation or the like.

The tail lamp 1501 includes the organic light emitting element of the present embodiment. The tail lamp 1501 may also include a protection member that protects the organic light emitting element. The protection member may be composed of any material that has a sufficiently high strength and is transparent, and can be composed of polycarbonate or the like. The polycarbonate may be mixed with a furandicarboxylic acid derivative, an acrylonitrile derivative, or the like.

The automobile 1500 may include a car body 1503 and a window 1502 attached to the car body 1503. The window 1502 may be a transparent display if not a window for checking the front and rear sides of the automobile. The transparent display includes an organic light emitting element of the present embodiment. In such a case, the materials constituting the electrodes, etc., of the organic light emitting element are transparent.

A moving body according to the present embodiment may be a ship, an airplane, a drone, or the like. The moving body may include a body and a lighting section attached to the body. The lighting section may emit light to indicate the position of the body. The lighting section includes an organic light emitting element of the present embodiment.

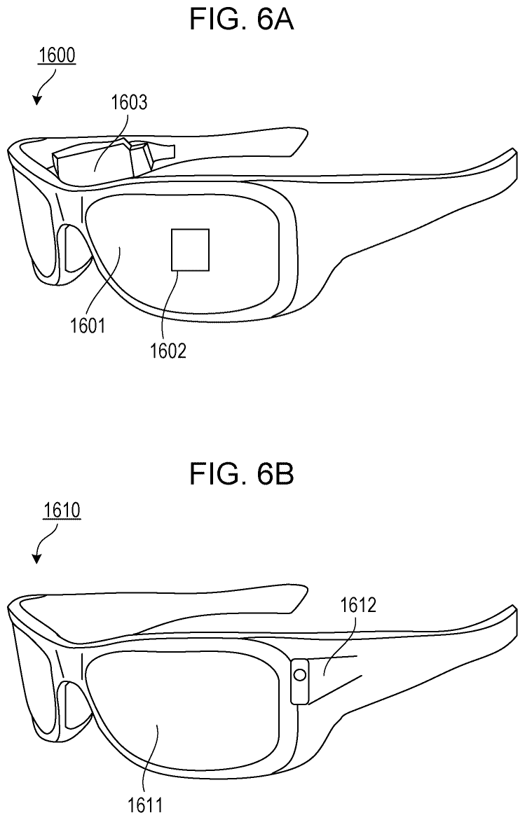

Referring now to FIGS. 6A and 6B, examples of applications of the display apparatuses of the aforementioned embodiments are described. The display apparatus can be applied to wearable device systems such as smart glasses, HMD, and smart contact lenses. An imaging display device used in such application examples includes an imaging apparatus capable of converting visible light into electricity, and a display apparatus capable of emitting visible light.

FIG. 6A is a schematic diagram of one example of a wearable device according to an embodiment of the present disclosure. FIG. 6A is used to explain glasses 1600 (smart glasses) according to one application example. An imaging apparatus 1602 such as a CMOS sensor or SPAD is disposed on a front side of a lens 1601 of the glasses 1600. The display apparatus according to any of the embodiments described above is disposed on a rear side of the lens 1601.

The glasses 1600 further include a controller 1603. The controller 1603 functions as a power supply that supplies power to the imaging apparatus 1602 and the display apparatus. The controller 1603 also controls the operation of the imaging apparatus 1602 and the display apparatus. An optical system for focusing light onto the imaging apparatus 1602 is formed in the lens 1601.

FIG. 6B is a schematic diagram of another example of a wearable device according to an embodiment of the present disclosure. FIG. 6B is used to explain glasses 1610 (smart glasses) according to one application example. The glasses 1610 include a controller 1612, and the controller 1612 includes an imaging apparatus that corresponds to the imaging apparatus 1602 illustrated in FIG. 6A, and a display apparatus. In a lens 1611, an optical system for projecting light emitted from the display apparatus and the imaging apparatus in the controller 1612 is formed, and images are projected onto the lens 1611. The controller 1612 functions as a power supply that supplies power to the imaging apparatus and the display apparatus, and also controls the operation of the imaging apparatus and the display apparatus.

The controller 1612 may include a line-of-sight detecting section that detects the line of sight of a wearer. The line of sight may be detected by using infrared radiation. An infrared radiation emitting section emits infrared radiation toward an eyeball of a user gazing the displayed image. A captured image of the eyeball is obtained when the imaging section having a light receiving element detects reflection of the emitted infrared radiation from the eyeball. The degradation of the image quality is decreased by providing a section for decreasing light from the infrared radiation emitting section to the display section in a plan view. The line of sight of the user with respect to the displayed image is detected from the captured image of the eyeball obtained by infrared imaging. Any known technique can be applied to the line-of-sight detection using the captured image of the eyeball. For example, a line-of-sight detection method based on a Purkinje image formed by reflection of the irradiated light at the cornea can be employed. More specifically, a line-of-sight detection process based on the pupil-corneal reflection method is performed. The line of sight of the user is detected by calculating the eye vector that indicates the direction (rotation angle) of the eyeball on the basis of the image of the pupil and the Purkinje image included in the captured image of the eyeball by using the pupil-corneal reflection method.

The display apparatus according to an embodiment of the present disclosure may include an imaging apparatus that includes a light receiving element, and may control the image displayed on the display apparatus on the basis of the line-of-sight information of the user from the imaging apparatus. Specifically, in the display apparatus, a first view region that the user gazes and a second view region other than the first view region are determined on the basis of the line-of-sight information. The first view region and the second view region may be determined by the controller in the display apparatus, or may be determined by an external controller and received. In the display region of the display apparatus, the display resolution of the first view region may be controlled to be higher than the display resolution of the second view region. In other words, the resolution of the second view region may be lower than that of the first view region.

The display region has a first display region and a second display region different from the first display region, and a region having a higher priority is determined from the first display region and the second display region on the basis of the line-of-sight information. The first view region and the second view region may be determined by the controller in the display apparatus, or may be determined by an external controller and received. The resolution of the region with higher priority may be controlled to be higher than the resolution of the region other than the region with higher priority. In other words, the resolution of a region having relatively low priority may be decreased.

An AI may be used to determine the first view region or the region with high priority. The AI may be a model configurated to estimate the angle of the line of sight and the distance to the object at the end of the line of sight from the image of the eyeball by using, as teaching data, the image of the eyeball and the direction in which the eyeball in the image was actually gazing. The AI program may be included in the display apparatus, the imaging apparatus, or an external apparatus. When an external apparatus includes the AI program, the data is transmitted to the display apparatus via communication.

When the display is controlled on the basis of the visual recognition, smart glasses that further include an imaging apparatus that captures the images of the outside can be implemented. The smart glasses can display the captured outside information in real time.

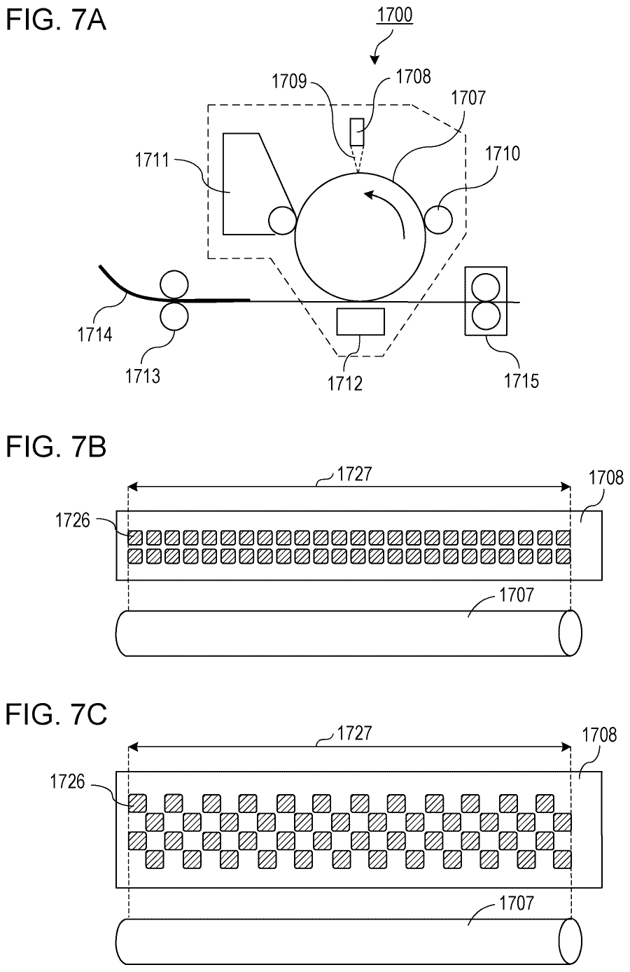

FIG. 7A is a schematic diagram of one example of an image forming apparatus according to an embodiment of the present disclosure. An image forming apparatus 1700 is an electrophotographic image forming apparatus and includes a photosensitive body 1707, an exposure light source 1708, a charging section 1710, a developing section 1711, a transferring device 1712, a conveying roller 1713, and a fixing device 1715. Light 1709 is emitted from the exposure light source 1708, and an electrostatic latent image is formed on a surface of the photosensitive body 1707. The light source 1708 includes an organic light emitting element of the present embodiment. The developing section 1711 includes a toner etc. The charging section 1710 charges the photosensitive body 1707. The transferring device 1712 transfers a developed image onto a recording medium 1714. The conveying roller 1713 conveys the recording medium 1714. The recording medium 1714 is, for example, a sheet of paper. The fixing device 1715 fixes the image formed on the recording medium 1714.

FIGS. 7B and 7C are schematic diagrams illustrating the exposure light source 1708 in which multiple light emitting sections 1726 are arranged on a long substrate. The arrow 1727 indicates the direction parallel to the axis of the photosensitive body as well as the direction in which rows of the organic light emitting elements are arranged. This row direction is coincident with the axis of rotation of the photosensitive body 1707. This direction can also be referred to as the longitudinal axis direction of the photosensitive body 1707. FIG. 7B illustrates an arrangement in which the light emitting sections 1726 are arranged along the longitudinal axis direction of the photosensitive body 1707. FIG. 7C illustrates an arrangement different from the one illustrated in FIG. 7B, in which the light emitting sections 1726 in the first row and the second row are staggered. The first row and the second row are at different positions in the column direction. The first row includes multiple light emitting sections 1726 with spaces therebetween. The second row includes light emitting sections 1726 at positions corresponding to the spaces between the light emitting sections 1726 in the first row. In other words, multiple light emitting sections 1726 are arranged to be spaced from each other in the column direction also. The arrangement illustrated in FIG. 7C can also be referred to as a grid arrangement state, a houndstooth pattern, or a checkered pattern.

As described heretofore, when an apparatus that uses the organic light emitting element of the present embodiment is used, good quality images can be displayed stably for a long time. In addition, when an apparatus that uses the organic light emitting element of the present embodiment is used, excellent outdoor visibility and power saving display can both be achieved due to highly efficient, high-luminance optical output.

### Ink Composition

Next, an ink composition according to an embodiment of the present disclosure is described. The ink composition of this embodiment contains at least one organic compound of the embodiment described above.

Since the organic compound of the present embodiment has good solubility in organic solvents, the organic compound can be used as an ink composition. Moreover, the organic compound layer constituting the organic light emitting element of the present embodiment, in particular, the light emitting layer, can be prepared by a coating method by using the ink composition of this embodiment; thus, large-area elements can be easily produced at relatively low cost.

Examples of solvents that dissolve the organic compound of the present embodiment include toluene, xylene, mesitylene, dioxane, methylnaphthalene, tetrahydrofuran, diglyme, 1,2-dichlorobenzene, and 1,2-dichloropropane. These organic solvents can be used alone or in combination of two or more. Of these, organic solvents having an appropriate evaporation rate, specifically, organic solvents having a boiling point of 70° C. to 200° C., may be used since thin films with even thickness are easily obtained.

The ink composition of the present embodiment may further contain compounds that serve as additives. Examples of compounds that can be used as additives include the aforementioned known light-emitting layer hosts and light-emitting assist materials, hole transport materials, light-emitting materials, and electron transport materials.

The concentration of the organic compound of the present embodiment in the ink composition relative to the entire composition may be 0.05 mass % or more and 20 mass % or less or may be 0.1 mass % or more and 5 mass % or less.

The ink composition of the present embodiment can be formed into a film by a spin coating method, a bar coating method, a slit coating method, an inkjet method, a nozzle coating method, a casting method, a gravure printing method, or the like. The organic light emitting element of the present embodiment can configure a display apparatus such as a display when a layer containing the organic compound of the present embodiment is formed on the electrode formed in the pixel pattern.

### EXAMPLES

Examples will now be described. These examples do not limit the scope of the present disclosure.

### Example 1

### (1) Synthesis of Exemplary Embodiment Compound 1 (Compound 1)

Exemplary embodiment compound 1 was synthesized via the following synthetic route. Before the reagent and the solvent were heated and stirred in each reaction step, argon bubbling was performed for 10 minutes.

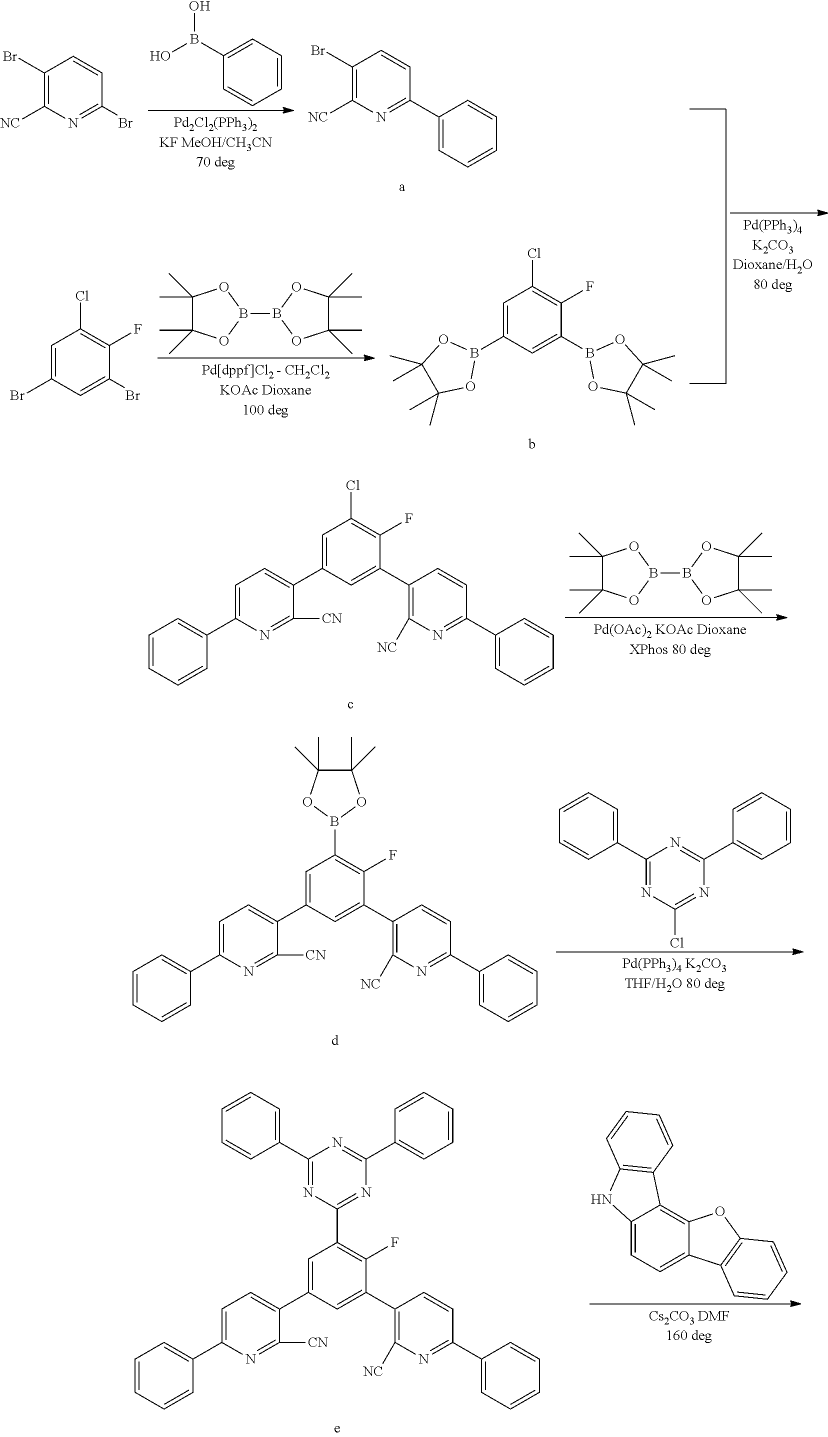

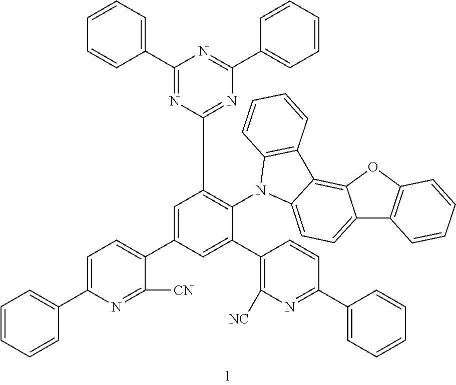 [SMILES: N#Cc1nc(-c2ccccc2)ccc1-c1cc(-c2nc(-c3ccccc3)nc(-c3ccccc3)n2)c(-n2c3ccccc3c3c4oc5ccccc5c4ccc32)c(-c2ccc(-c3ccccc3)nc2C#N)c1]

### (1-1) Synthesis of Intermediate a

Into a flask, 3,6-dibromopicolinonitrile (3.1 g), phenylboronic acid (1.6 g), dichlorobis(triphenylphosphine)palladium(II) (0.42 g), potassium fluoride (3.8 g), acetonitrile (40 mL), and water (13 mL) were added and stirred at 70° C. for 11 hours. After extraction with ethyl acetate and drying over magnesium sulfate, the solid matter was removed by filtration and the resulting mixture was concentrated. The resulting product was purified by column chromatography using an ethyl acetate-hexane (volume ratio 3:7) mixed solution as a developing solvent, as a result of which 1.93 g of a white powder was obtained. The peak m/z=258.0 of the intermediate 1 was confirmed by GCMS.

### (1-2) Synthesis of Intermediate b

Dioxane (48 mL) was added to 1,5-dibromo-3-chloro-2-fluorobenzene (2.3 g), bis(pinacolato)diboron (5.1 g), [1,1′-bis(diphenylphosphino)ferrocene]palladium(II) dichloride dichloromethane adduct (0.33 g), and potassium acetate (9.8 g), and the resulting mixture was stirred at 100° C. for 11 hours. The resulting mixture was concentrated at reduced pressure, hexane was added thereto, and the resulting mixture was subjected to celite filtration to thereby obtain 3.8 g of a sticky substance. The peak m/z=382.2 of the intermediate 2 was confirmed by GCMS. The area ratios of other GCMS peaks revealed that this sticky substance contained 66% of the intermediate b. In the subsequent synthesis, this sticky substance was used as was.

### (1-3) Synthesis of Intermediate c

Dioxane (20 mL) and water (5 mL) were added to the intermediate a (0.87 g), the intermediate b (0.61 g), tetrakistriphenylphosphine palladium (92 mg), and potassium carbonate (2.0 g), and the resulting mixture was stirred at 80° C. for 11 hours. Water was added to the reaction mixture, the resulting mixture was extracted with ethyl acetate and dried over magnesium sulfate, and then celite filtration was performed and the filtrate was evaporated to dryness. The resulting product was purified by column chromatography using an ethyl acetate-hexane (volume ratio 4:6) mixture as a developing solvent, as a result of which 0.51 g of a white powder was obtained. Furthermore, gel permeation chromatography was performed with chloroform as a developing solvent to thereby obtain 0.24 g of the product. The peak m/z=486.1 of the intermediate c was confirmed by GCMS.

### (1-4) Synthesis of Intermediate d

Dioxane (5 mL) was added to the intermediate c (490 mg), bis(pinacolato)diboron (480 mg), palladium acetate (4.5 mg), potassium acetate (294 mg), and XPhos (2-dicyclohexylphosphino-2′,4′,6′-triisopropylbiphenyl) (19 mg), and the resulting mixture was stirred at 110° C. for 11 hours. Water was added to the reaction mixture, the resulting mixture was extracted with ethyl acetate and dried over magnesium sulfate, and then celite filtration was performed and the resulting product was evaporated to dryness at reduced pressure to thereby obtain 490 mg of a gray powder. The peak m/z=578.2 of the intermediate d was confirmed by GCMS.

### (1-5) Synthesis of Intermediate e

Tetrahydrofuran (12 mL) and water (4 mL) were added to the intermediate d (310 mg), 2-chloro-4,6-diphenyl-1,3,5-triazine (155 mg), tetrakistriphenylphosphine palladium (6 mg), and potassium carbonate (220 mg), and the resulting mixture was stirred at 80° C. for 11 hours. To the reaction mixture, 30 mL of chloroform was added to conduct extraction, and the resulting product was dried over magnesium sulfate, filtered through celite, and concentrated. Thereto, 20 mL of chloroform was added, methanol was added to the resulting mixture to deposit a white solid matter, and filtration was performed to obtain 350 mg of a white powder. The peak m/z=578.2 of the intermediate e was confirmed by GCMS.

### (1-6) Synthesis of Exemplary Embodiment Compound 1

N,N-dimethylformamide (10 mL) was added to the intermediate e (300 mg), 5H-benzofuro[3,2-c]carbazole (120 mg), and cesium carbonate (280 mg), and the resulting mixture was stirred at 160° C. for 11 hours. To this reaction solution, chloroform and water were added to extract the reaction product into an organic layer. The organic layer was filtered through celite and concentrated at reduced pressure. The residue was purified by silica gel column chromatography (solvent: toluene), methanol was added to the resulting solution, and the deposited solid matter was filtered to thereby obtain 150 mg of a pale yellow powder. The HPLC purity was 98.0%.

### (2) Synthesis of Exemplary Embodiment Compound 2 (Compound 3)

Exemplary embodiment compound 2 was synthesized via the following synthetic route.

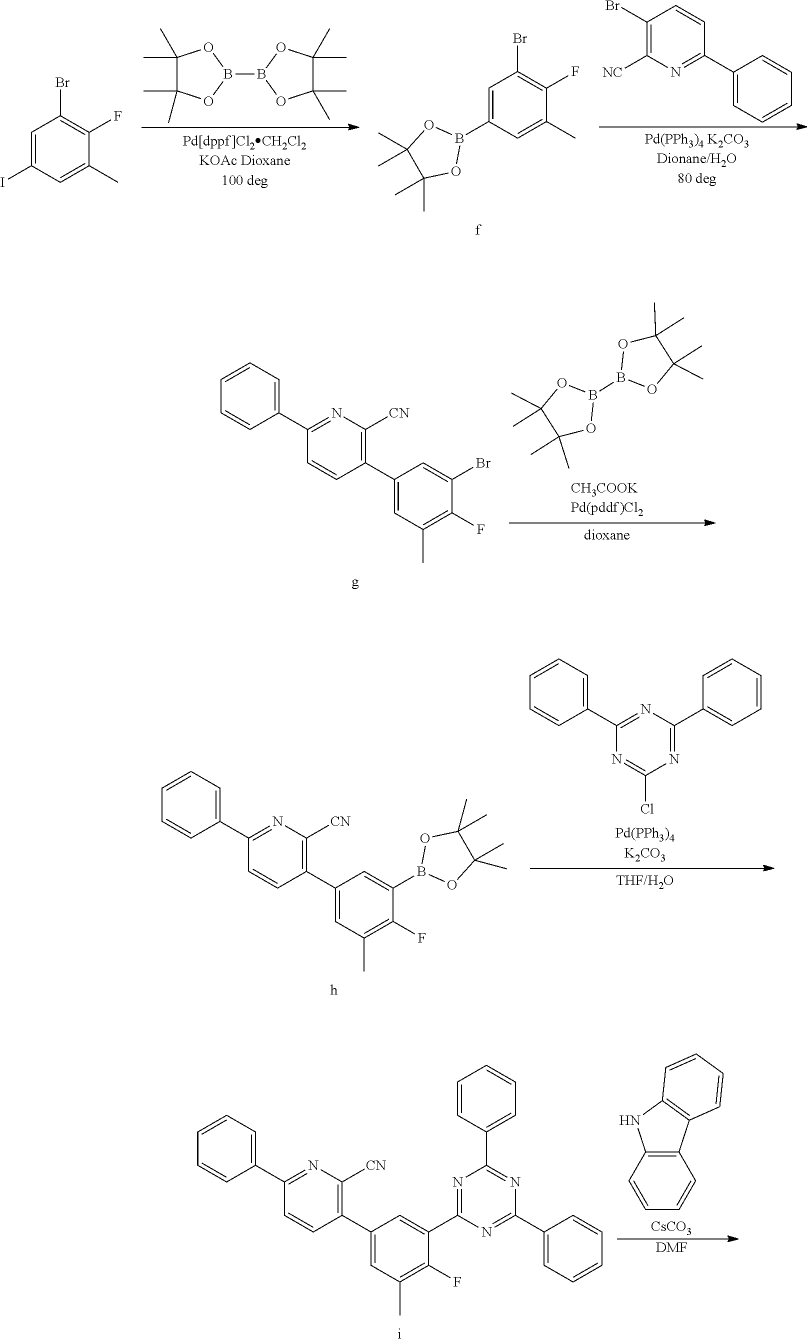

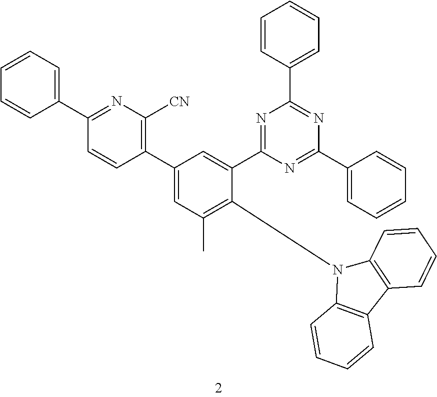 [SMILES: Cc1cc(-c2ccc(-c3ccccc3)nc2C#N)cc(-c2nc(-c3ccccc3)nc(-c3ccccc3)n2)c1-n1c2ccccc2c2ccccc21]

### (2-1) Synthesis of Intermediate g

Dioxane (16 mL) was added to 1-bromo-2-fluoro-5-iodo-3-methylbenzene (5.0 g), bis(pinacolato)diboron (4.4 g), [1,1′-bis(diphenylphosphino)ferrocene]palladium(II) dichloride (0.42 g), and potassium acetate (4.7 g), and the resulting mixture was stirred at 100° C. for 11 hours to obtain a reaction mixture containing an intermediate f After the reaction mixture was cooled to room temperature, the intermediate a (4.5 g), a 2 M aqueous sodium carbonate solution (39.0 mL), bis(triphenylphosphine)palladium(II) dichloride (Pd(PPh₃)₂Cl₂, 884 mg), and dioxane (7.9 mL) were added to the reaction mixture, and the resulting mixture was stirred at 80° C. for 11 hours. Ethyl acetate was added to the reaction mixture, and the resulting mixture was extracted with ethyl acetate and dried over magnesium sulfate. The solvent was removed, and the residue was purified by column chromatography (hexane/chloroform) to thereby obtain an intermediate g (3.6 g).

### (2-2) Synthesis of Intermediate i

Dioxane (11 mL) was added to the intermediate g (3.6 g), bis(pinacolato)diboron (3.3 g), [1,1′-bis(diphenylphosphino)ferrocene]palladium(II) dichloride (0.30 g), and potassium acetate (3.4 g), and the resulting mixture was stirred at 100° C. for 11 hours. As a result, a reaction mixture containing an intermediate h was obtained. After the reaction mixture was cooled to room temperature, 2-chloro-4,6-diphenyl-1,3,5-triazine (3.4 g), a 2 M aqueous sodium carbonate solution (28.0 mL), bis(triphenylphosphine)palladium(II) dichloride (Pd(PPh₃)₂Cl₂, 402 mg), and THF (17 mL) were added thereto, and the resulting mixture was stirred at 80° C. for 11 hours. Ethyl acetate was added to the reaction mixture, and the resulting mixture was extracted with ethyl acetate and dried over magnesium sulfate. The solvent was removed, and the residue was purified by column chromatography (hexane/dichloromethane) to thereby obtain an intermediate i (3.9 g).

### (2-3) Synthesis of Exemplary Embodiment Compound 2

DMF (19.2 mL) was added to the intermediate 4 (1.5 g), 9H-carbazole (2.7 g), and cesium carbonate (5.6 g) in a nitrogen atmosphere, and the resulting mixture was stirred at 150° C. for 11 hours. The resulting mixture was returned to room temperature, water and methanol were then added to the reaction mixture, and the resulting suspension was filtered. The residue was purified by recycling preparative GPC (JAI-GEL, chloroform) to obtain exemplary embodiment compound 2 (1.5 g) as a pale yellow solid.

### (3) Synthesis of Exemplary Embodiment Compound 3 (Compound 12)

According to the synthesis method of exemplary embodiment compound 1, exemplary embodiment compound 3 was synthesized by the following reaction scheme from 1,5-dibromo-2,4-difluoro-3-methylbenzene as a starting material.

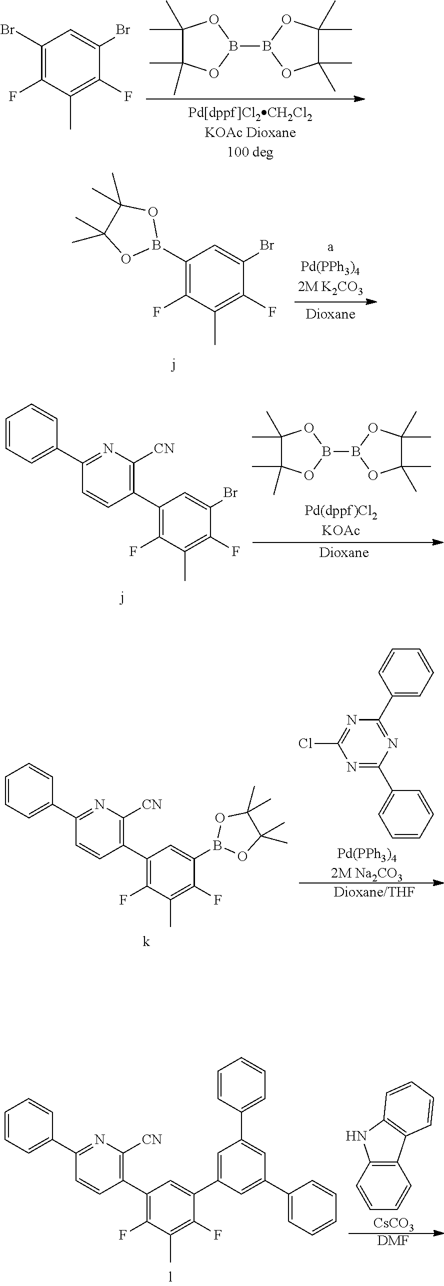

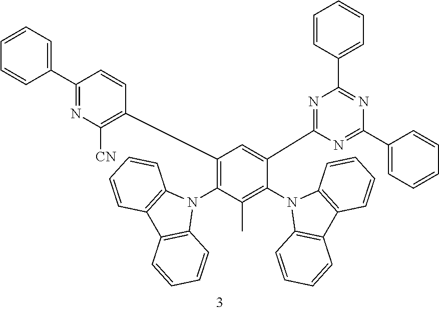 [SMILES: Cc1c(-n2c3ccccc3c3ccccc32)c(-c2nc(-c3ccccc3)nc(-c3ccccc3)n2)cc(-c2ccc(-c3ccccc3)nc2C#N)c1-n1c2ccccc2c2ccccc21]

### (4) Synthesis of Exemplary Embodiment Compound 4 (Compound 14)

Exemplary embodiment compound 4 was synthesized via the following synthetic route.

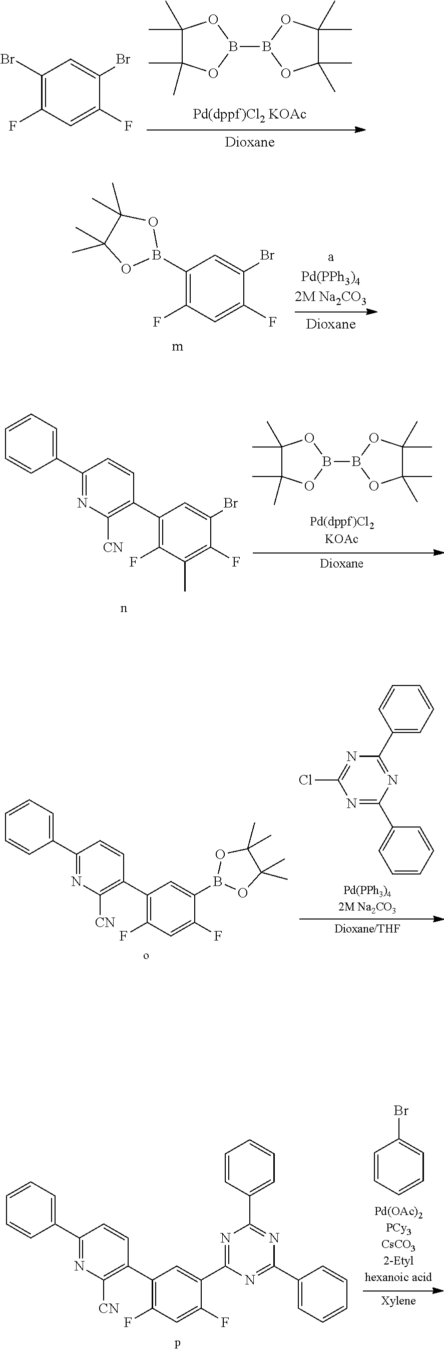

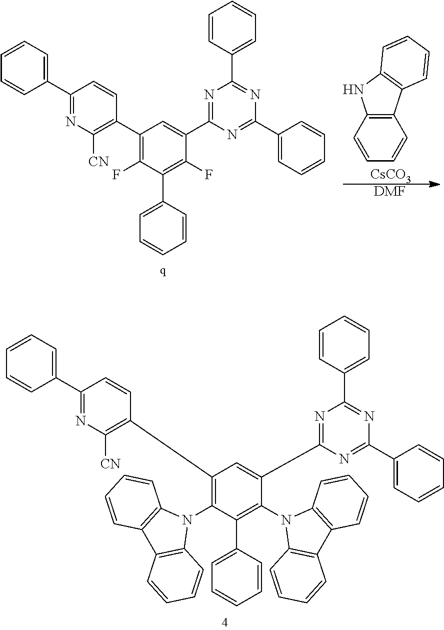

### (4-1) Synthesis of Intermediates m to p

The intermediates m to p were synthesized according to the aforementioned synthetic route by using 1,5-dibromo-2,4-difluorobenzene as a starting material.

### (4-2) Synthesis of Intermediate q

Xylene (23 mL) was added to palladium(II) acetate (46 mg), the intermediate p (1.2 g), bromobenzene (1.8 g), cesium carbonate (2.6 g), 2-ethylhexanoic acid (0.6 g), and tricyclohexylphosphine (0.17 g) in a nitrogen atmosphere, and the resulting mixture was stirred at 130° C. for 11 hours. To the reaction mixture, chloroform was added, and the resulting mixture was extracted with chloroform, dried over magnesium sulfate, and had the solvent removed by distillation. The residue was purified by recycling preparative GPC (JAI-GEL, chloroform) to obtain an intermediate q (0.33 g).

### (4-3) Synthesis of Exemplary Embodiment Compound 4

Synthesis was conducted according to the synthetic route of exemplary embodiment compound 1 but with the intermediate q.

### (5) Synthesis of Exemplary Embodiment Compound 5 (Compound 17)

Exemplary embodiment compound 5 was synthesized via the following synthetic route.

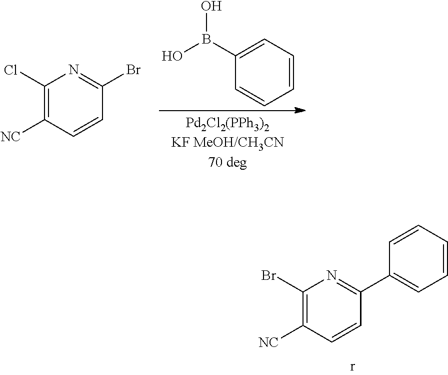 [SMILES: N#Cc1ccc(Br)nc1Cl ; OB(O)c1ccccc1 ; N#Cc1ccc(-c2ccccc2)nc1Br]

### (5-1) Synthesis of Intermediate r

An intermediate r was synthesized by using 6-bromo-2-chloronicotinonitrile as a raw material according to the synthetic route of the intermediate a.

### (5-2) Synthesis of Exemplary Embodiment Compound 5

Exemplary embodiment compound 5 was synthesized according to the synthetic route of the exemplary embodiment compound 3 except that the intermediate r was used instead of the intermediate a.

### (6) Synthesis of Exemplary Embodiment Compound 6 (Compound 19)

Exemplary embodiment compound 6 was synthesized according to the synthetic route of the exemplary embodiment compound 4 except that the intermediate r was used instead of the intermediate a.

### (7) Synthesis of Exemplary Embodiment Compound 7 (Compound 7)

Exemplary embodiment compound 7 was synthesized according to the synthetic route of the exemplary embodiment compound 1 except that the intermediate r was used instead of the intermediate a and that 3-phenyl-9H-carbazole was used instead of 5H-benzofuro[3,2-c]carbazole.

### (8) Synthesis of Exemplary Embodiment Compound 8 (Compound 5)

Exemplary embodiment compound 5 was synthesized via the following synthetic route.

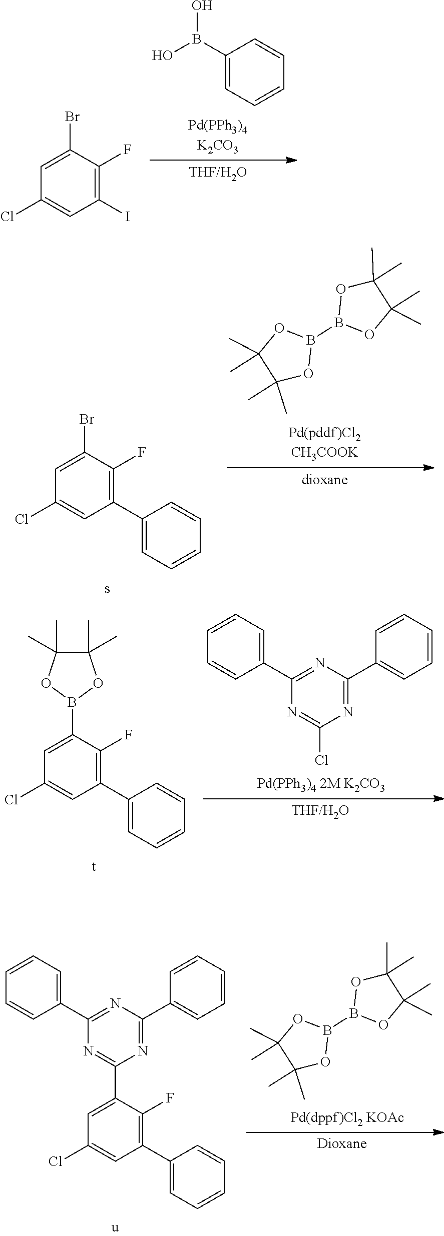

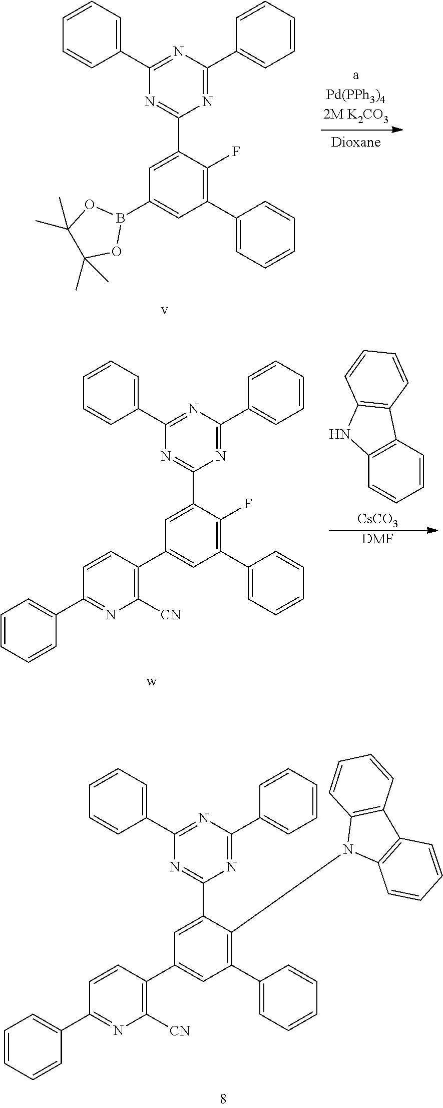

### (8-1) Synthesis of Intermediate s

In a nitrogen atmosphere, toluene (75 mL) was added to 1-bromo-5-chloro-2-fluoro-3-iodobenzene (5.00 g), phenylboronic acid (2.09 g), a 2 M aqueous sodium carbonate solution (22.4 mL), and tetrakis(triphenylphosphine)palladium(0) (1.12 g), and the resulting mixture was stirred at 80° C. for 11 hours. Ethyl acetate was added to the reaction mixture, and the resulting mixture was extracted with ethyl acetate and dried over magnesium sulfate. The solvent was removed, and the residue was purified by column chromatography (hexane) to thereby obtain an intermediate s (3.41 g).

### (8-2) Synthesis of Intermediates t and u

The intermediates t and u were synthesized by the same method as that for the intermediate i by using the intermediate s.

### (8-3) Synthesis of Intermediates v and w

The intermediates v and w were synthesized by the same method as that for the intermediate h by using the intermediate u.

### (8-4) Synthesis of Exemplary Embodiment Compound 8

Exemplary embodiment compound 8 was synthesized by the same method as that for the exemplary embodiment compound 2 but with the intermediate w.

### (9) Synthesis of Exemplary Embodiment Compound 9 (Compound 22)

Exemplary embodiment compound 9 was synthesized according to the synthetic route of the exemplary embodiment compound 2 by using the intermediate i and 3-phenyl-9H-carbazole.

### (10) Synthesis of Exemplary Embodiment Compound 10 (Compound 9)

Exemplary embodiment compound 10 was synthesized according to the synthetic route of the exemplary embodiment compound 2 except that the intermediate r was used instead of the intermediate a.

### (11) Synthesis of Exemplary Embodiment Compound 11 (Compound 11)

Exemplary embodiment compound 11 was synthesized according to the synthetic route of the exemplary embodiment compound 8 except that the intermediate r was used instead of the intermediate a.

### (12) Synthesis of Exemplary Embodiment Compound 12 (Compound 15)

Exemplary embodiment compound 12 was synthesized via the following synthetic route.

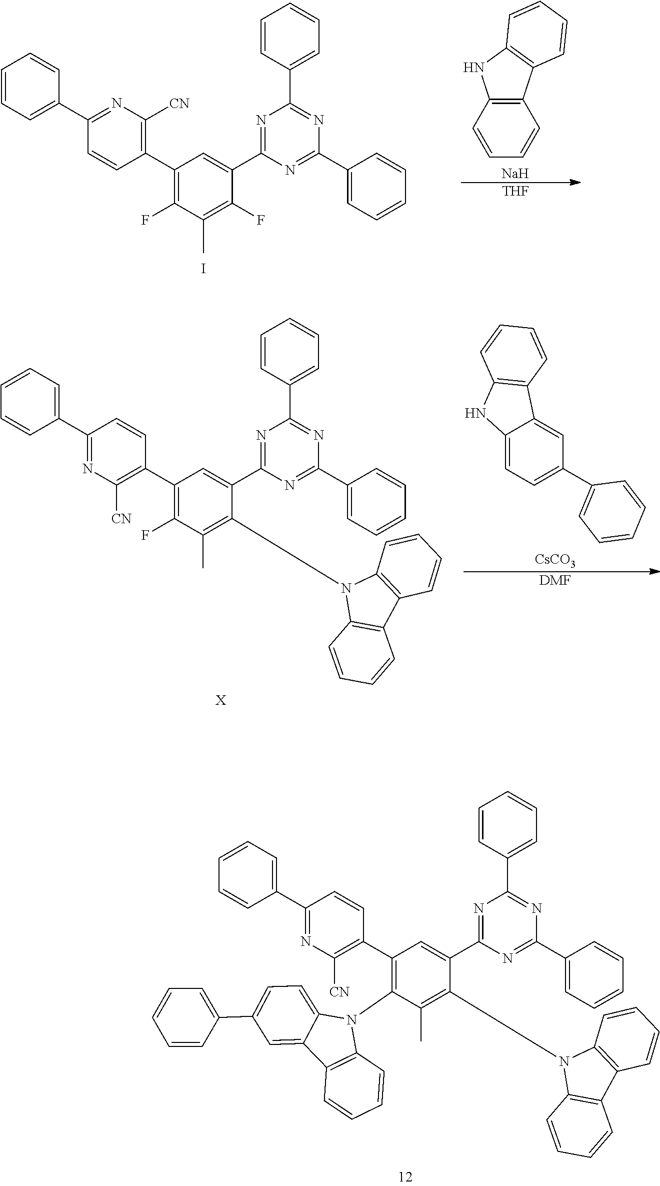

### (12-1) Synthesis of Intermediate x

THF (19 mL) was added to 9H-carbazole (0.17 g) and sodium hydride (50 mass %, 0.13 g) in a nitrogen atmosphere, followed by stirring at 0° C. for 1 hour, and the resulting reaction mixture was cooled to −78° C., the intermediate 1 (0.50 g) was added thereto, and the resulting mixture was stirred for 24 hours. Water was added to the reaction solution, and the resulting mixture was concentrated with a rotary evaporator. The suspension was filtered, and the residue was purified by column chromatography (hexane/toluene) to thereby obtain an intermediate w (0.38 g) as a pale yellow solid.

### (12-2) Synthesis of Exemplary Embodiment Compound 12

Exemplary embodiment compound 12 was synthesized according to the synthetic route of the exemplary embodiment compound 2 by using the intermediate w and 3-phenyl-9H-carbazole.

### (13) Synthesis of Exemplary Embodiment Compound 13 (Compound 20)

Exemplary embodiment compound 13 was synthesized according to the synthetic route of the exemplary embodiment compound 12 except that the intermediate r was used instead of the intermediate a.

### (14) Exemplary Embodiment Compound 14 (Compound 25)

Exemplary embodiment compound 14 was synthesized according to the synthetic route of the exemplary embodiment compound 10 except that 3-phenyl-9H-carbazole was used instead of 9H-carbazole.

### (14) Synthesis of Exemplary Embodiment Compound 15 (Compound 8)

Exemplary embodiment compound 15 was synthesized according to the synthetic route of the exemplary embodiment compound 7 except that 5H-benzofuro[3,2-c]carbazole was used instead of 9H-carbazole.

### (15) Synthesis of Comparative Example Compounds 1 to 3

Comparative example compound 1 was synthesized according to the synthetic route of exemplary embodiment compound 3 but through the following reaction scheme using the intermediate q.

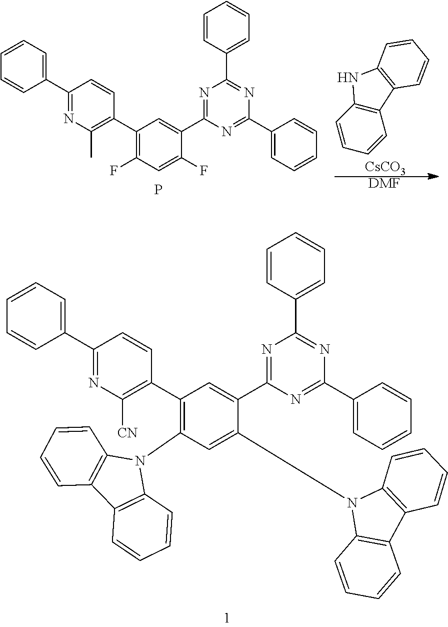

Comparative example compounds 2 and 3 were synthesized according to the synthetic method disclosed in International Publication No. 2023/140374. Comparative example compounds 1 to 3 are indicated below.

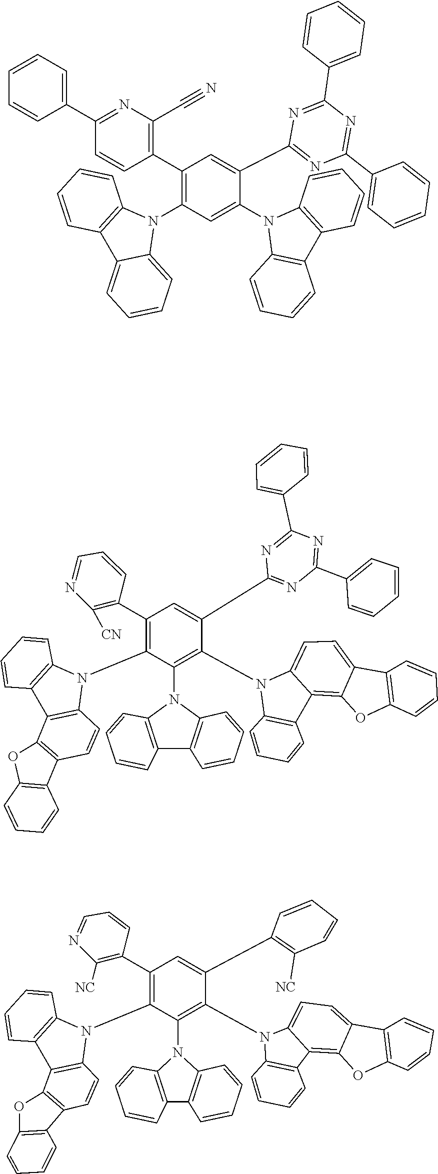 [SMILES: N#Cc1nc(-c2ccccc2)ccc1-c1cc(-c2nc(-c3ccccc3)nc(-c3ccccc3)n2)c(-n2c3ccccc3c3ccccc32)cc1-n1c2ccccc2c2ccccc21 ; N#Cc1ncccc1-c1cc(-c2nc(-c3ccccc3)nc(-c3ccccc3)n2)c(-n2c3ccccc3c3c4oc5ccccc5c4ccc32)c(-n2c3ccccc3c3ccccc32)c1-n1c2ccccc2c2c3oc4ccccc4c3ccc21 ; N#Cc1ccccc1-c1cc(-c2cccnc2C#N)c(-n2c3ccccc3c3c4oc5ccccc5c4ccc32)c(-n2c3ccccc3c3ccccc32)c1-n1c2ccccc2c2c3oc4ccccc4c3ccc21]

### Comparative Example Compound 1 Comparative Example Compound 2 Comparative Example Compound 3

### Example 2

For the exemplary embodiment compounds 1 to 15 and the comparative example compounds 1 to 3 synthesized in Example 1, the sublimation temperature and ΔEST were measured by the following methods. Table 2 indicates the molecular weight, the measured value of ΔEST, and the sublimation temperature of each of the compounds.

### Method for Measuring Sublimation Temperature

2.0 mg of each compound was weighed, and the temperature at which the weight decreased 10% under vacuum TG measurement was assumed to be the sublimation temperature.

### Method for Measuring ΔEST

After Ar bubbling a 1.0×10⁻⁵ M toluene solution for 5 minutes, the emission spectrum of the fluorescence at room temperature (298 K) was measured. A tangent line was drawn with respect to the short-wave-side rise of the measured fluorescence spectrum, the wave λF [nm] at the intersection between the tangent line and the horizontal axis was determined, and the excited singlet energy level S₁ [eV] was calculated from the equation, excited singlet energy level [eV]=1239.85/λF. A prepared sample was cooled to 77 K, and the emission spectrum of phosphorescence measured after blocking the excited light was measured in the same manner. A tangent line was drawn with respect to the short-wave-side rise of the measured phosphorescence spectrum, the wave XP [nm] at the intersection between the tangent line and the horizontal axis was determined, and the excited triplet energy level T₁ [eV] was calculated from the equation, excited triplet energy level [eV]=1239.85/λP. T₁ was subtracted from S₁ measured as described above to calculate ΔEST.

TABLE 2

| | | | Sublimation | ΔEST |
|---|---|---|---|---|
| | Compound | Mw | temperature [°C] | [eV] |
| Example 1 | Compound 1 | 921 | 83 | 0.01 |
| Example 2 | Compound 3 | 667 | 78 | 0.10 |
| Example 3 | Compound 12 | 832 | 86 | 0.04 |
| Example 4 | Compound 14 | 894 | 91 | 0.06 |
| Example 5 | Compound 17 | 832 | 84 | 0.10 |
| Example 6 | Compound 19 | 894 | 91 | 0.05 |
| Example 7 | Compound 7 | 907 | 93 | 0.10 |
| Example 8 | Compound 5 | 729 | 84 | 0.09 |
| Example 9 | Compound 22 | 743 | 83 | 0.08 |
| Example 10 | Compound 9 | 667 | 78 | 0.09 |
| Example 11 | Compound 11 | 729 | 83 | 0.09 |
| Example 12 | Compound 15 | 908 | 94 | 0.04 |
| Example 13 | Compound 20 | 908 | 94 | 0.10 |
| Example 14 | Compound 25 | 743 | 84 | 0.08 |
| Example 15 | Compound 8 | 921 | 95 | 0.10 |
| Comparative | Comparative example | 818 | 87 | 0.14 |
| Example 1 | compound 1 | | | |
| Comparative | Comparative example | 1087 | 100 | 0.06 |
| Example 2 | compound 2 | | | |
| Comparative | Comparative example | 954 | 96 | 0.05 |
| Example 3 | compound 3 | | | |

Table 2 indicates that the exemplary embodiment compounds 1 to 5 have small ΔEST and low sublimation temperatures, and compared with Comparative Examples 2 and 3, the sublimation temperatures of the exemplary embodiment compounds 1 to 5 are low.

ΔEST is smaller in the exemplary embodiment compounds than in comparative example 1. This demonstrates that it is better to have a substituent in R₂ of general formula (1). Comparison between the exemplary embodiment compounds 3 and 5 and exemplary embodiment compounds 8 and 11 reveals that ΔEST is smaller when A in general formula (1) is substituted with (2-1) than when A in general formula (1) is substituted with (2-2). Comparison between the exemplary embodiment compound 3 and the exemplary embodiment compound 4 reveals that R₂ may be an alkyl group rather than an aryl group to decrease the sublimation temperature.

In sum, the organic compounds according to the present disclosure have small ΔEST and low sublimation temperature. Moreover, when the organic compound of the present embodiment is used in an organic light emitting element, an organic light emitting element having excellent element durability can be provided.

According to the present disclosure, an organic compound with small ΔEST and low sublimation temperature can be provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-017637, filed Feb. 5, 2025, which is hereby incorporated by reference herein in its entirety.

What is claimed is:

1. An organic compound represented by formula (1): 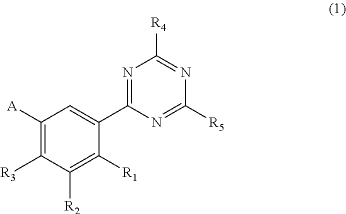
where, in formula (1),
A is a group represented by formula (2-1) or (2-2),
R₁ is a group represented by formula (3) or (4-1),
R₂ is a substituted or unsubstituted alkyl group, a substituted or unsubstituted aryl group, a substituted or unsubstituted heteroaryl group, or a group represented by formula (2-1) or (2-2),
R₃ is a hydrogen atom or a group represented by formula (3),
when R₂ is a group represented by formula (2-1) or (2-2), R₁ is a group represented by formula (3) or (4-1) and R₃ is a hydrogen atom,
when R₂ is an alkyl group, an aryl group, or a heteroaryl group, R₁ is a group represented by formula (3) and R₃ is a hydrogen atom or a group represented by formula (3),
R₄ and R₅ are each independently a substituted or unsubstituted aryl group, a substituted or unsubstituted alkyl group, or a substituted or unsubstituted heteroaryl group, 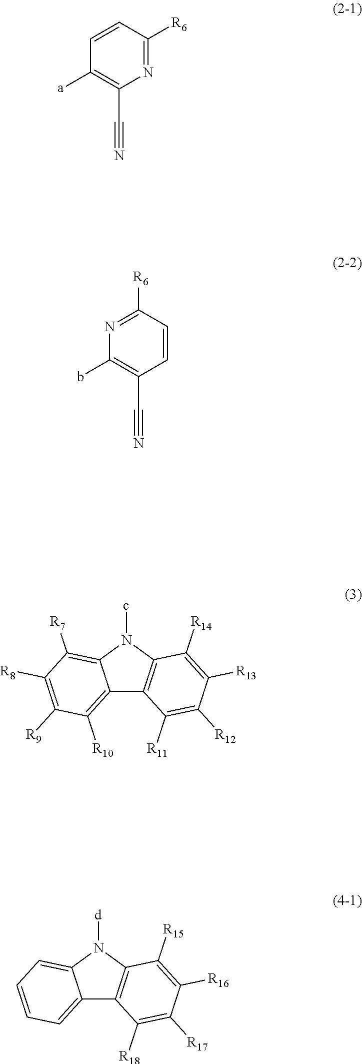
in formulae (2-1) and (2-2), R₆ is a substituted or unsubstituted alkyl group, a substituted or unsubstituted aryl group, or a substituted or unsubstituted heteroaryl group,
in formula (3), R₇ to R₁₄ are each independently selected from a hydrogen atom, a deuterium atom, a halogen atom, a substituted or unsubstituted alkyl group, a substituted or unsubstituted aryl group, a substituted or unsubstituted heterocyclic group, a substituted or unsubstituted amino group, a substituted or unsubstituted alkoxy group, a substituted or unsubstituted aryloxy group, a substituted or unsubstituted heteroaryloxy group, a substituted or unsubstituted silyl group, and a cyano group,
in formula (3), among R₁₁ to R₁₄, R₁₁ and R₁₂, R₁₂ and R₁₃, or R₁₃ and R₁₄ optionally bond to * in formula (4-2), 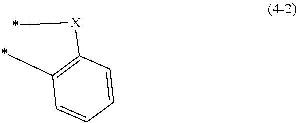
in formula (4-2),
X is selected from an oxygen atom, a sulfur atom, a selenium atom, and a tellurium atom, and
in formulae (2-1), (2-2), (3), and (4-1), a, b, c, and d each represent a bonding site to formula (1).

2. The organic compound according to claim 1, wherein, in formula (1), A is a group represented by formula (2-1), and R₂ is an alkyl group having 1 or more and 4 or less carbon atoms.

3. The organic compound according to claim 1, wherein, in formula (3), at least five of R₇ to R₁₄ are hydrogen atoms.

4. The organic compound according to claim 1, wherein, in formula (4-2), X is an oxygen atom or a sulfur atom.

5. The organic compound according to claim 1, wherein, in formula (4-1), R₁₇ and R₁₈ bond to a group represented by formula (4-2).

6. The organic compound according to claim 1, wherein R₄ to R₁₄ are each independently selected from a hydrogen atom, a deuterium atom, and groups represented by structural formulae: 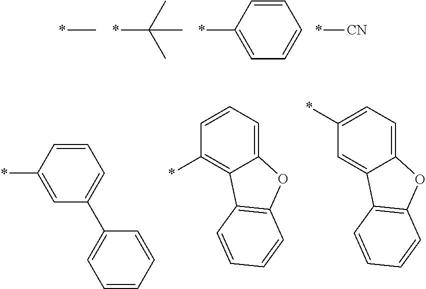
where * represents a bonding site.

7. The organic compound according to claim 1, wherein R₂ is an alkyl group having 1 or more and 4 or less carbon atoms or an aryl group having 6 carbon atoms.

8. The organic compound according to claim 1, wherein R₁ and R₃ are each a group represented by formula (3).

9. The organic compound according to claim 8, wherein R₁ and R₃ are each independently selected from groups represented by structural formulae:

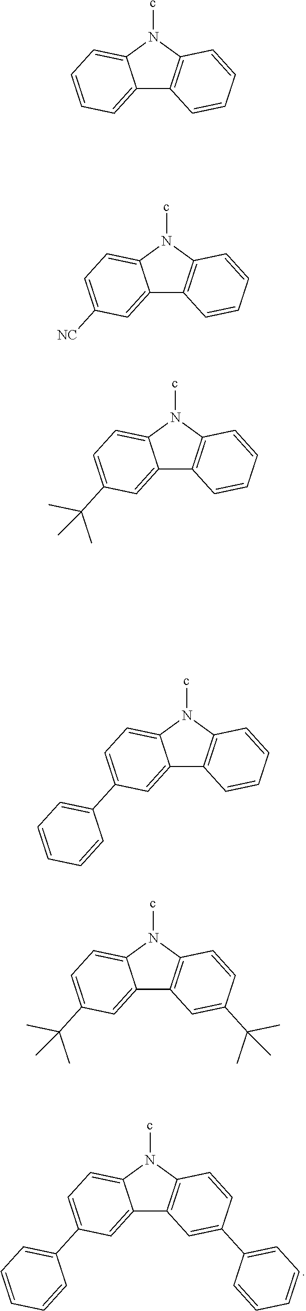

10. The organic compound according to claim 1, wherein, among R₁₅ to R₁₈ in formula (4-1), R₁₅ and R₁₆, R₁₆ and R₁₇, or R₁₇ and R₁₈ bond to * in formula (4-2), and the remainder are hydrogen atoms.

11. The organic compound according to claim 1, wherein R₄ and R₅ are each an aryl group.

12. The organic compound according to claim 1, having a molecular weight of 950 or less.

13. An organic light emitting element comprising:
a first electrode;
a second electrode; and
an organic compound layer disposed between the first electrode and the second electrode,
wherein the organic compound layer includes at least one layer containing the organic compound according to claim 1.

14. The organic light emitting element according to claim 13, wherein the layer containing the organic compound is a light emitting layer.

15. The organic light emitting element according to claim 14, wherein the light emitting layer contains a second organic compound different from the organic compound.

16. An ink composition comprising the organic compound according to claim 1.

17. A display apparatus comprising:
a plurality of pixels,
wherein at least one of the plurality of pixels includes the organic light emitting element according to claim 13 and a transistor connected to the organic light emitting element.

18. A photoelectric conversion apparatus comprising:
an optical section that includes a plurality of lenses;
an imaging element that receives light that has passed through the optical section; and
a display section that displays an image captured by the imaging element,
wherein the display section includes the organic light emitting element according to claim 13.

19. Electronic equipment comprising:
a display section that includes the organic light emitting element according to claim 13;
a casing that accommodates the display section; and
a communication section that is disposed in the casing and performs external communication.

20. A moving body comprising:
a lighting section that includes the organic light emitting element according to claim 13; and
a body having the lighting section.
